(19) United States
(12) Patent Application Publication
(10) Pub. No.: US 2006/0281908 A1
(43) Pub. Date: Dec. 14, 2006

(54) Xylose isomerases, nucleic acids encoding them and methods for making and using them

(76) Inventor: Walter Callen, San Diego, CA (US)

Correspondence Address: DIVERSA C/O MOFO S.D., 12531 HIGH BLUFF DRIVE, SUITE 100, SAN DIEGO, CA, 92130-2040, US

(21) Appl. No.: 10/533,241

(22) PCT Filed: Oct. 23, 2003
(86) PCT No.: PCT/US03/34008

§ 371 Date: Jul. 21, 2006

Related U.S. Application Data
(60) Provisional application No. 60/424,649, filed on Nov. 6, 2002.

(51) Int. Cl. C12Q 1/68; C07H 21/04; C12P 21/06; C12P 19/02; C12N 9/92; C12N 1/21

(52) U.S. Cl. 536/23.2; 435/234; 435/105; 435/69.1; 435/252.3; 435/320.1; 435/6

(57) ABSTRACT

In one aspect, the invention provides xylose isomerase enzymes, polynucleotides encoding the enzymes, methods of making and using these polynucleotides and polypeptides. The polypeptides of the invention can be used in a variety of agricultural and industrial contexts. For example, the polypeptides of the invention can be used for converting glucose to fructose or for manufacturing high content fructose syrups in large quantities. Other examples include use of the polypetides of the invention in confectionary, brewing, alcohol and soft drinks production, and in diabetic foods and sweetners.

### TECHNICAL FIELD

This invention relates to molecular and cellular biology and biochemistry. In one aspect, the invention provides xylose isomerase enzymes, polynucleotides encoding the enzymes, methods of making and using these polynucleotides and polypeptides. The polypeptides of the invention can be used in a variety of agricultural and industrial contexts. For example, the polypeptides of the invention can be used for converting glucose to fructose or for manufacturing high content fructose syrups in large quantities. Other examples include use of the polypeptides of the invention in confectionary, brewing, alcohol and soft drinks production, and in diabetic foods and sweeteners.

### BACKGROUND

D-xylose isomerase, also called D-xylose ketol isomerase or glucose isomerase, catalyzes the reversible isomerization of D-xylose to D-xylulose in the first step of xylose metabolism following the pentose phosphate cycle. It also catalyzes the reversible isomerization of D-glucose into D-fructose. Xylose isomerase is widely used in industry for the production of high-fructose syrup.

Xylose isomerases can catalyze the conversion of D-xylose to an equilibrium mixture of D-xylulose and D-xylose. When supplied with cobalt ions these xylose isomerases were found to isomerize α-D-glucopyranose to α-D-fructofuranose, equilibration from the more abundant β-D-glucopyranose and to the major product β-D-fructopyranose occurring naturally and non-enzymatically. Several genera of microbes, mainly bacteria such as Actinoplanes missouriensis, Bacillus coagulans and various Streptomyces species, can produce a glucose isomerase that have specificities for glucose and fructose which are not much different from that for xylose.

### SUMMARY

The invention provides isolated or recombinant nucleic acids comprising a nucleic acid sequence having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to an exemplary nucleic acid of the invention. In one aspect, the invention provides an isolated or recombinant nucleic acid comprising a nucleic acid sequence having at least 96%, 97%, 98%, 99%, or more, or complete (100%) sequence identity to SEQ ID NO:1 or SEQ ID NO:5 over a region of at least about 100 residues, or a nucleic acid sequence having at least 95%, 96%, 97%, 98%, 99%, or more, or complete (100%) sequence identity to SEQ ID NO:3 over a region of at least about 100 residues, wherein the nucleic acid encodes at least one polypeptide having a xylose isomerase activity, and the sequence identities are determined by analysis with a sequence comparison algorithm or by a visual inspection.

In alternative aspects, the nucleic acid sequence has at least 96%, 97%, 98%, 99% or more or complete (100%) sequence identity to SEQ ID NO:1 or SEQ ID NO:5 over a region of at least about 150, 200, 250, 300, 350, 400, 450, 500, 550, 600, 650, 700, 750, 800, 850, 900, 950, 1000, 1050, 1100, 1150 or more residues, or, a nucleic acid sequence having at least 95%, 96%, 97%, 98%, 99% or more or complete (100%) sequence identity sequence identity to SEQ ID NO:3 over a region of at least about 150, 200, 250, 300, 350, 400, 450, 500, 550, 600, 650, 700, 750, 800, 850, 900, 950, 1000, 1050, 1100, 1150 or more residues. In alternative aspects, the nucleic acid sequence comprises a nucleic acid having a sequence as set forth in SEQ ID NO:1, SEQ ID NO:3, SEQ ID NO:5 or subsequences thereof. In alternative aspects, the nucleic acid sequence encodes a polypeptide having a sequence as set forth in SEQ ID NO:2, SEQ ID NO:4, SEQ ID NO:6, or subsequences thereof.

In one aspect, the invention provides a xylose isomerase where one amino acid was changed from SEQ ID NO:2, from MTEFFPEI . . . (in SEQ ID NO:2) to MAEFFPEI . . . (SEQ ID NO:6), which is also active in isomerizing glucose and fructose. The first nucleotide residue in the coding sequence for SEQ ID NO:6 (the coding sequence designated SEQ ID NO:5) after the first codon ATG was changed to a “G” to provide a restriction site for cloning to enzyme coding sequence. In one aspect, SEQ ID NO:5 is used to overexpress the enzyme.

In one aspect, the sequence comparison algorithm is a BLAST version 2.2.2 algorithm where a filtering setting is set to blastall -p blastp -d “nr pataa” -F F, and all other options are set to default.

The xylose isomerases of the invention, and the xylose isomerase-encoding nucleic acids of the invention, have a common novelty in that were initially derived from a common source, i.e., an environmental source.

In one aspect, the xylose isomerase activity comprises isomerization of xylose to xylulose, or the reverse reaction. In one aspect, the xylose isomerase activity comprises isomerization of glucose to fructose, or the reverse reaction. In alternative aspects, the xylose isomerase activity comprises the isomerization of a D-glucose to a D-fructose, or, the xylose isomerase activity comprises catalysis of the conversion of D-xylose to an equilibrium mixture of D-xylulose and D-xylose, or, the xylose isomerase activity comprises isomerization of α-D-glucopyranose to α-D-fructofuranose, or, the xylose isomerase activity comprises isomerization of β-D-glucopyranose to β-D-fructopyranose, or the reverse reactions.

In another aspect, the isolated or recombinant nucleic acid encodes a polypeptide having a xylose isomerase activity which is thermotolerant. The polypeptide can retain a xylose isomerase activity after exposure to a temperature in the range from greater than 37° C. to about 95° C. or anywhere in the range from greater than 55° C. to about 85° C. The polypeptide can retain a xylose isomerase activity after exposure to a temperature in the range between about 1° C. to about 5° C., between about 5° C. to about 15° C., between about 15° C. to about 25° C., between about 25° C. to about 37° C., between about 37° C. to about 95° C., between about 55° C. to about 85° C., between about 70° C. to about 75° C., or between about 90° C. to about 95° C., or more. In one aspect, the polypeptide retains a xylose isomerase activity after exposure to a temperature in the range from greater than 90° C. to about 95° C. at pH 4.5. In one aspect, a polypeptide of the invention retains a xylose isomerase activity after exposure to conditions comprising a temperature range of between about 95° C. to about 135° C., or, between about 95° C. to about 105° C., or it retains a xylose isomerase activity after exposure to conditions comprising a temperature range of between about 105° C. to about 120° C., or, between about 120° C. to about 135° C.

In one aspect, the isolated or recombinant nucleic acid encodes a polypeptide having a xylose isomerase activity which is thermostable. In one aspect, the polypeptide has xylose isomerase activity at a temperature in the range from greater than 37° C. to about 95° C. or anywhere in the range from greater than 55° C. to about 85° C. The polypeptide has xylose isomerase activity at a temperature in the range between about 1° C. to about 5° C., between about 5° C. to about 15° C., between about 15° C. to about 25° C., between about 25° C. to about 37° C., between about 37° C. to about 95° C., between about 55° C. to about 85° C., between about 70° C. to about 75° C., or between about 90° C. to about 95° C., or more. In one aspect, the polypeptide has xylose isomerase activity at a temperature in the range from greater than 90° C. to about 95° C. at pH 4.5. In one aspect, a polypeptide of the invention has xylose isomerase activity at a temperature range of between about 95° C. to about 135° C., or, between about 95° C. to about 105° C., or it retains a xylose isomerase activity after exposure to conditions comprising a temperature range of between about 105° C. to about 120° C., or, between about 120° C. to about 135° C.

The invention provides an isolated or recombinant nucleic acid, wherein the nucleic acid comprises a sequence that hybridizes under stringent conditions to a nucleic acid comprising a sequence as set forth in SEQ ID NO:1, SEQ ID NO:3, or SEQ ID NO:5, or subsequences thereof, wherein the nucleic acid encodes a polypeptide having a xylose isomerase activity. The nucleic acid can be at least about 15, 20, 25, 30, 35, 40, 45, 50, 55, 60, 70, 80, 90, 100, 125, 150, 175, 200, 225, 250, 275, 300, 325, 350, 400, 450, 500, 550, 600, 650, 700, 750, 800, 850, 900, 950, 1000, 1050, 1100, 1150 or more residues in length, or the full length of a gene or a transcript. In one aspect, the stringent conditions include a wash step comprising a wash in 0.2×SSC at a temperature of about 65° C. for about 15 minutes.

The invention provides a nucleic acid probe for identifying a nucleic acid encoding a polypeptide comprising a xylose isomerase activity, wherein the probe comprises at least 10 consecutive bases of a sequence of the invention, e.g., a sequence as set forth in SEQ ID NO:1, SEQ ID NO:3, or SEQ ID NO:5, wherein the probe identifies the nucleic acid by binding or hybridization. The probe can comprise an oligonucleotide comprising at least about 10 to 50, about 20 to 60, about 30 to 70, about 40 to 80, or about 60 to 100 consecutive bases of a sequence of the invention, e.g., a sequence as set forth in SEQ ID NO:1, SEQ ID NO:3, or SEQ ID NO:5.

The invention provides a nucleic acid probe for identifying a nucleic acid encoding a polypeptide having a xylose isomerase activity, wherein the probe comprises a nucleic acid comprising a sequence of the invention, e.g., a sequence as set forth in SEQ ID NO:1, SEQ ID NO:3, or SEQ ID NO:5, or, a nucleic acid sequence having at least 96% sequence identity to SEQ ID NO:1 over a region of at least about 100 residues, or a nucleic acid sequence having at least 95% sequence identity to SEQ ID NO:3 over a region of at least about 100 residues, wherein the sequence identities are determined by analysis with a sequence comparison algorithm or by visual inspection. The nucleic acid probe can comprise an oligonucleotide comprising at least about 10 to 50, about 20 to 60, about 30 to 70, about 40 to 80, or about 60 to 100 consecutive bases of a nucleic acid sequence as set forth in SEQ ID NO:1, or a subsequence thereof, a sequence as set forth in SEQ ID NO:3, or a subsequence thereof. In one aspect, the nucleic acid probe comprises a nucleic acid sequence having at least 97%, 98%, 99%, or more sequence identity to a region of at least about 100 residues of a nucleic acid comprising a sequence as set forth in SEQ ID NO:1 or SEQ ID NO:3 or subsequences thereof.

The invention provides an amplification primer sequence pair for amplifying a nucleic acid encoding a polypeptide having a xylose isomerase activity, wherein the primer pair is capable of amplifying a nucleic acid comprising a sequence as set forth in SEQ ID NO:1, SEQ ID NO:3, or SEQ ID NO:5 or subsequences thereof. The amplification primer pair can comprise an oligonucleotide comprising at least about 10, 15, 20, 25 30, 35, 40, 45 to 50, 60, 70 or more consecutive bases of the sequence. One or each member of the amplification primer sequence pair can comprise an oligonucleotide comprising at least about 10 to 50 consecutive bases of the sequence, or about 12, 13, 14, 15, 16, 17, 18, 19, 20, 21, 22, 23, 24, 25, 26, 27, 28, 29, 30 or more consecutive bases of the sequence.

The invention provides a method for amplifying a nucleic acid encoding a polypeptide having a xylose isomerase activity comprising amplification of a template nucleic acid with an amplification primer sequence pair capable of amplifying a nucleic acid sequence as set forth in SEQ ID NO:1, SEQ ID NO:3, or SEQ ID NO:5 or subsequences thereof.

The invention provides amplification primer pairs, wherein the primer pair comprises a first member having a sequence as set forth by about the first (the 5′) 12, 13, 14, 15, 16, 17, 18, 19, 20, 21, 22, 23, 24, 25, 26, 27, 28, 29, 30 or more residues of a nucleic acid of the invention, and a second member having a sequence as set forth by about the first (the 5′) 12, 13, 14, 15, 16, 17, 18, 19, 20, 21, 22, 23, 24, 25, 26, 27, 28, 29, 30 or more residues of the complementary strand of the first member.

The invention provides xylose isomerase-encoding nucleic acids generated by amplification, e.g., polymerase chain reaction (PCR), using an amplification primer pair of the invention. The invention provides xylose isomerases generated by amplification, e.g., polymerase chain reaction (PCR), using an amplification primer pair of the invention. The invention provides methods of making a xylose isomerase by amplification, e.g., polymerase chain reaction (PCR), using an amplification primer pair of the invention. In one aspect, the amplification primer pair amplifies a nucleic acid from a library, e.g., a gene library, such as an environmental library.

The invention provides methods of amplifying a nucleic acid encoding a polypeptide having xylose isomerase activity comprising amplification of a template nucleic acid with an amplification primer sequence pair capable of amplifying a nucleic acid sequence of the invention, or fragments or subsequences thereof.

The invention provides an expression cassette comprising a nucleic acid of the invention, e.g., a nucleic acid comprising: (i) a nucleic acid sequence having at least 96% sequence identity to SEQ ID NO:1 over a region of at least about 100 residues, or a nucleic acid sequence having at least 95% sequence identity to SEQ ID NO:3 over a region of at least about 100 residues, wherein the sequence identities are determined by analysis with a sequence comparison algorithm or by a visual inspection; or, (ii) a nucleic acid that hybridizes under stringent conditions to a nucleic acid comprising a sequence as set forth in SEQ ID NO:1 or SEQ ID NO:3 or subsequences thereof. The nucleic acid can be operably linked to a plant promoter. The expression cassette can further comprise a plant expression vector. The plant expression vector can comprise a plant virus. The plant promoter can comprise a potato promoter, a rice promoter, a corn promoter, a wheat promoter or a barley promoter. The promoter can comprises a promoter derived from T-DNA of Agrobacterium tumefaciens. The promoter can be a constitutive promoter or an inducible promoter or a tissue-specific promoter, developmentally regulated or environmentally regulated promoter, such as a seed-specific, a leaf-specific, a root-specific, a stem-specific or an abscission-induced promoter.

The invention provides a vector comprising a nucleic acid of the invention, e.g., a nucleic acid comprising (i) a nucleic acid sequence having at least 96% sequence identity to SEQ ID NO:1 over a region of at least about 100 residues, or a nucleic acid sequence having at least 95% sequence identity to SEQ ID NO:3 over a region of at least about 100 residues, wherein the sequence identities are determined by analysis with a sequence comparison algorithm or by a visual inspection; or, (ii) a nucleic acid that hybridizes under stringent conditions to a nucleic acid comprising a sequence as set forth in SEQ ID NO:1 or SEQ ID NO:3 or subsequences thereof.

The invention provides a cloning vehicle comprising a vector of the invention or a nucleic acid of the invention. The cloning vehicle can comprise a viral vector, a plasmid, a phage, a phagemid, a cosmid, a fosmid, a bacteriophage or an artificial chromosome. The viral vector can comprise an adenovirus vector, a retroviral vector or an adeno-associated viral vector. The cloning vehicle can comprise a bacterial artificial chromosome (BAC), a plasmid, a bacteriophage P1-derived vector (PAC), a yeast artificial chromosome (YAC), or a mammalian artificial chromosome (MAC).

The invention provides a transformed cell comprising a vector of the invention, e.g., a vector comprising (i) a nucleic acid sequence having at least 96% sequence identity to SEQ ID NO:1 over a region of at least about 100 residues, or a nucleic acid sequence having at least 95% sequence identity to SEQ ID NO:3 over a region of at least about 100 residues, wherein the sequence identities are determined by analysis with a sequence comparison algorithm or by a visual inspection; or, (ii) a nucleic acid that hybridizes under stringent conditions to a nucleic acid comprising a sequence as set forth in SEQ ID NO:1 or SEQ ID NO:3 or subsequences thereof.

The invention provides a transformed cell comprising a nucleic acid of the invention, e.g., a nucleic acid comprising (i) a nucleic acid sequence having at least 96% sequence identity to SEQ ID NO:1 over a region of at least about 100 residues, or a nucleic acid sequence having at least 95% sequence identity to SEQ ID NO:3 over a region of at least about 100 residues, wherein the sequence identities are determined by analysis with a sequence comparison algorithm or by a visual inspection; or, (ii) a nucleic acid that hybridizes under stringent conditions to a nucleic acid comprising a sequence as set forth in SEQ ID NO:1 or SEQ ID NO:3 or subsequences thereof. In one aspect, the cell is a bacterial cell, a mammalian cell, a fungal cell, a yeast cell, an insect cell or a plant cell. The transformed cell can be any plant cell, such as a potato, rice, corn, wheat, tobacco, rapeseed, grass, soybean or barley cell.

The invention provides a transgenic non-human animal comprising a nucleic acid of the invention, e.g., a nucleic acid comprising (i) a nucleic acid sequence having at least 96% sequence identity to SEQ ID NO:1 over a region of at least about 100 residues, or a nucleic acid sequence having at least 95% sequence identity to SEQ ID NO:3 over a region of at least about 100 residues, wherein the sequence identities are determined by analysis with a sequence comparison algorithm or by a visual inspection; or, (ii) a nucleic acid that hybridizes under stringent conditions to a nucleic acid comprising a sequence as set forth in SEQ ID NO:1 or SEQ ID NO:3 or subsequences thereof. The transgenic non-human animal can be any non-human animal, e.g., a mouse.

The invention provides a transgenic plant comprising a nucleic acid of the invention, e.g., a nucleic acid comprising (i) a nucleic acid sequence having at least 96% sequence identity to SEQ ID NO:1 over a region of at least about 100 residues, a nucleic acid sequence having at least 95% sequence identity to SEQ ID NO:3 over a region of at least about 100 residues, wherein the sequence identities are determined by analysis with a sequence comparison algorithm or by a visual inspection; or, (ii) a nucleic acid that hybridizes under stringent conditions to a nucleic acid comprising a sequence as set forth in SEQ ID NO:1 or SEQ ID NO:3 or subsequences thereof. The transgenic plant can be any plant, such as a corn plant, a potato plant, a grass, a tomato plant, a wheat plant, an oilseed plant, a rapeseed plant, a soybean plant or a tobacco plant.

The invention provides a method of making a transgenic plant comprising the following steps: (a) introducing a heterologous nucleic acid sequence into the cell, wherein the heterologous nucleic sequence comprises a sequence of the invention, thereby producing a transformed plant cell; (b) producing a transgenic plant from the transformed cell.

The invention provides a transgenic seed comprising a nucleic acid of the invention, e.g., a nucleic acid comprising (i) a nucleic acid sequence having at least 96% sequence identity to SEQ ID NO:1 over a region of at least about 100 residues, or a nucleic acid sequence having at least 95% sequence identity to SEQ ID NO:3 over a region of at least about 100 residues, wherein the sequence identities are determined by analysis with a sequence comparison algorithm or by a visual inspection; or, (ii) a nucleic acid that hybridizes under stringent conditions to a nucleic acid comprising a sequence as set forth in SEQ ID NO:1 or SEQ ID NO:3 or subsequences thereof. The transgenic seed can be any seed or equivalent structure, such as a starch granule or grain, corn seed, a wheat kernel, an oilseed, a rapeseed, a soybean seed, a palm kernel, a sunflower seed, a sesame seed, a peanut or a tobacco plant seed.

The invention provides an antisense oligonucleotide comprising a nucleic acid of the invention, e.g., a nucleic acid comprising a sequence complementary to or capable of hybridizing under stringent conditions to (i) a nucleic acid sequence having at least 96% sequence identity to SEQ ID NO:1 over a region of at least about 100 residues, or a nucleic acid sequence having at least 95% sequence identity to SEQ ID NO:3 over a region of at least about 100 residues, wherein the sequence identities are determined by analysis with a sequence comparison algorithm or by a visual inspection; or, (ii) a nucleic acid that hybridizes under stringent conditions to a nucleic acid comprising a sequence as set forth in SEQ ID NO:1 or SEQ ID NO:3 or subsequences thereof. The antisense oligonucleotide can be any length, e.g., between about 10 to 50, about 20 to 60, about 30 to 70, about 40 to 80, or about 60 to 100 bases in length, or any variation thereof.

The invention provides a method of inhibiting the translation of a xylose isomerase message in a cell comprising administering to the cell or expressing in the cell an antisense oligonucleotide of the invention, e.g., an antisense oligonucleotide comprising a nucleic acid sequence complementary to or capable of hybridizing under stringent conditions to a nucleic acid comprising (i) a nucleic acid sequence having at least 96% sequence identity to SEQ ID NO:1 over a region of at least about 100 residues, or a nucleic acid sequence having at least 95% sequence identity to SEQ ID NO:3 over a region of at least about 100 residues, wherein the sequence identities are determined by analysis with a sequence comparison algorithm or by a visual inspection; or, (ii) a nucleic acid that hybridizes under stringent conditions to a nucleic acid comprising a sequence as set forth in SEQ ID NO:1 or SEQ ID NO:3 or subsequences thereof.

The invention provides methods of inhibiting the translation of a xylose isomerase message in a cell comprising administering to the cell or expressing in the cell an antisense oligonucleotide comprising a nucleic acid sequence complementary to or capable of hybridizing under stringent conditions to a nucleic acid of the invention. The invention provides double-stranded inhibitory RNA (RNAi) molecules comprising a subsequence of a sequence of the invention. In one aspect, the RNAi is about 15, 16, 17, 18, 19, 20, 21, 22, 23, 24, 25 or more duplex nucleotides in length. The invention provides methods of inhibiting the expression of a xylose isomerase in a cell comprising administering to the cell or expressing in the cell a double-stranded inhibitory RNA (iRNA), wherein the RNA comprises a subsequence of a sequence of the invention.

The invention provides an isolated or recombinant polypeptide comprising an amino acid sequence having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to an exemplary polypeptide or peptide of the invention over a region of at least about 25, 50, 75, 100, 125, 150, 175, 200, 225, 250, 275, 300, 325, 350 or more residues, or over the full length of the polypeptide, and the sequence identities are determined by analysis with a sequence comparison algorithm or by a visual inspection. Exemplary polypeptide or peptide sequences of the invention include SEQ ID NO:2; SEQ ID NO:4; SEQ ID NO:6, and peptides and fragments thereof.

In one aspect, the invention provides an isolated or recombinant polypeptide comprising (a) a polypeptide comprising an amino acid sequence having at least 96%, 97%, 98%, 99%, or more, or complete (100%) sequence identity to SEQ ID NO:2 or SEQ ID NO:6 over a region of at least about 100 residues, or an amino acid sequence having at least 95%, 96%, 97%, 98%, 99%, or more, or complete (100%) sequence identity to SEQ ID NO:4 over a region of at least about 100 residues, or (b) a polypeptide encoded by a nucleic acid of the invention, e.g., a nucleic acid comprising (i) a nucleic acid sequence having at least 96% sequence identity to SEQ ID NO:1 or SEQ ID NO:5 over a region of at least about 100 residues, or a nucleic acid sequence having at least 95% sequence identity to SEQ ID NO:3 over a region of at least about 100 residues, wherein the sequence identities are determined by analysis with a sequence comparison algorithm or by a visual inspection; or, (ii) a nucleic acid that hybridizes under stringent conditions to a nucleic acid comprising a sequence as set forth in SEQ ID NO:1 or SEQ ID NO:3 or subsequences thereof. In one aspect, the polypeptide comprises a xylose isomerase activity.

The isolated or recombinant polypeptide can have an amino acid sequence having at least 96%, 97%, 98%, 99% or more identity to SEQ ID NO:2 or SEQ ID NO:6 over a region of at least about 150, 200, 250, 300, 350, 400 or more residues, or the full length of the protein, or, an amino acid sequence having at least 95%, 96%, 97%, 98%, 99% or more identity to SEQ ID NO:4 over a region of at least about 150, 200, 250, 300, 350, 400 or more residues, or the full length of the protein.

In alternative aspects, a xylose isomerase activity of a polypeptide of the invention comprises: isomerization of xylose to xylulose; isomerization of glucose to fructose; isomerization of a D-glucose to a D-fructose; catalysis of the conversion of D-xylose to an equilibrium mixture of D-xylulose and D-xylose; isomerization of β-D-glucopyranose to β-D-fructopyranose; and/or, isomerization of α-D-glucopyranose to α-D-fructofuranose, or, isomerization of xylulose to xylose; isomerization of fructose to glucose; isomerization of a D-fructose to D-glucose; catalysis of the conversion of an equilibrium mixture of D-xylulose and D-xylose to D-xylose; isomerization of β-D-fructopyranose to β-D-glucopyranose; and/or, isomerization of α-D-fructofuranose to α-D-glucopyranose.

In another aspect, the polypeptide of the invention has a xylose isomerase activity which is thermotolerant. The polypeptide can retain a xylose isomerase activity after exposure to a temperature in the range from greater than 37° C. to about 95° C. or anywhere in the range from greater than 55° C. to about 85° C. The polypeptide can retain a xylose isomerase activity after exposure to a temperature in the range between about 1° C. to about 5° C., between about 5° C. to about 15° C., between about 15° C. to about 25° C., between about 25° C. to about 37° C., between about 37° C. to about 95° C., between about 55° C. to about 85° C., between about 70° C. to about 75° C., or between about 90° C. to about 95° C., or more. In one aspect, the polypeptide retains a xylose isomerase activity after exposure to a temperature in the range from greater than 90° C. to about 95° C. at pH 4.5. In one aspect, a polypeptide of the invention retains a xylose isomerase activity after exposure to conditions comprising a temperature range of between about 95° C. to about 135° C., or, between about 95° C. to about 105° C., or it retains a xylose isomerase activity after exposure to conditions comprising a temperature range of between about 105° C. to about 120° C., or, between about 120° C. to about 135° C.

In one aspect, the polypeptide of the invention has a xylose isomerase activity which is thermostable. In one aspect, the polypeptide has xylose isomerase activity at a temperature in the range from greater than 37° C. to about 95° C. or anywhere in the range from greater than 55° C. to about 85° C. The polypeptide has xylose isomerase activity at a temperature in the range between about 1° C. to about 5° C., between about 5° C. to about 15° C., between about 15° C. to about 25° C., between about 25° C. to about 37° C., between about 37° C. to about 95° C., between about 55° C. to about 85° C., between about 70° C. to about 75° C., or between about 90° C. to about 95° C., or more. In one aspect, the polypeptide has xylose isomerase activity at a temperature in the range from greater than 90° C. to about 95° C. at pH 4.5. In one aspect, a polypeptide of the invention has xylose isomerase activity at a temperature range of between about 95° C. to about 135° C., or, between about 95° C. to about 105° C., or it retains a xylose isomerase activity after exposure to conditions comprising a temperature range of between about 105° C. to about 120° C., or, between about 120° C. to about 135° C.

Another aspect of the invention provides an isolated or recombinant polypeptide or peptide including at least 10, 15, 20, 25, 30, 35, 40, 45, 50, 55, 60, 65, 70, 75, 80, 85, 90, 95 or 100 or more consecutive bases of a polypeptide or peptide sequence of the invention, sequences substantially identical thereto, and the sequences complementary thereto. The peptide can be, e.g., an immunogenic fragment a motif (e.g., a binding site), a signal sequence, a prepro sequence or an active site. These peptides can act as signal sequences on its endogenous protease, on another protease, or a heterologous protein (a non-protease enzyme or other protein).

In one aspect, the invention provides a protein comprising a polypeptide of the invention lacking a signal sequence. In one aspect, the isolated or recombinant polypeptide can comprise the polypeptide of the invention comprising a heterologous signal sequence, such as a heterologous xylose isomerase or non-xylose isomerase signal sequence.

In one aspect, the invention provides a signal sequence comprising a peptide comprising/consisting of a sequence as set forth in residues 1 to 12, 1 to 13, 1 to 14, 1 to 15, 1 to 16, 1 to 17, 1 to 18, 1 to 19, 1 to 20, 1 to 21, 1 to 22, 1 to 23, 1 to 24, 1 to 25, 1 to 26, 1 to 27, 1 to 28, 1 to 28, 1 to 30, 1 to 31, 1 to 32, 1 to 33, 1 to 34, 1 to 35, 1 to 36, 1 to 37, 1 to 38, 1 to 39, 1 to 40, 1 to 41, 1 to 42, 1 to 43, 1 to 44 (or a longer peptide) of a polypeptide of the invention. In one aspect, the invention provides a signal sequence comprising a peptide comprising/consisting of a sequence as set forth SEQ ID NO:2, SEQ ID NO:4, or SEQ ID NO:6. In one aspect, the invention provides an isolated or recombinant signal sequence peptide comprising/consisting of a sequence as set forth in the amino terminal 20 to 30 residues of a polypeptide of the invention, e.g., SEQ ID NO:2, SEQ ID NO:4 or SEQ ID NO:6.

In one aspect, the invention provides chimeric proteins comprising a first domain comprising a signal sequence of the invention and at least a second domain. The protein can be a fusion protein. The second domain can comprise an enzyme. The enzyme can be a xylose isomerase.

The invention provides chimeric polypeptides comprising at least a first domain comprising signal peptide (SP), a prepro sequence and/or a catalytic domain (CD) of the invention and at least a second domain comprising a heterologous polypeptide or peptide, wherein the heterologous polypeptide or peptide is not naturally associated with the signal peptide (SP), prepro sequence and/or catalytic domain (CD). In one aspect, the heterologous polypeptide or peptide is not a xylose isomerase. The heterologous polypeptide or peptide can be amino terminal to, carboxy terminal to or on both ends of the signal peptide (SP), prepro sequence and/or catalytic domain (CD).

The invention provides isolated or recombinant nucleic acids encoding a chimeric polypeptide, wherein the chimeric polypeptide comprises at least a first domain comprising signal peptide (SP), a prepro domain and/or a catalytic domain (CD) of the invention and at least a second domain comprising a heterologous polypeptide or peptide, wherein the heterologous polypeptide or peptide is not naturally associated with the signal peptide (SP), prepro domain and/or catalytic domain (CD).

In alternative aspects, the xylose isomerase activity comprises a specific activity at about 95° C. in the range from about 100 to about 1000 units per milligram of protein, or, a specific activity from about 500 to about 750 units per milligram of protein, or, a specific activity at 95° C. in the range from about 500 to about 1200 units per milligram of protein, or, a specific activity at 95° C. in the range from about 750 to about 1000 units per milligram of protein. In one aspect, the xylose isomerase comprises a specific activity at about 37° C. in the range from about 1 to about 1200 units per milligram of protein, or, about 100 to about 1000 units per milligram of protein. In another aspect, xylose isomerase activity comprises a specific activity from about 100 to about 1000 units per milligram of protein, or, from about 500 to about 750 units per milligram of protein. Alternatively, the xylose isomerase activity comprises a specific activity at 37° C. in the range from about 1 to about 750 units per milligram of protein, or, from about 500 to about 1200 units per milligram of protein. In one aspect, xylose isomerase activity comprises a specific activity at 37° C. in the range from about 1 to about 500 units per milligram of protein, or, from about 750 to about 1000 units per milligram of protein. In another aspect, xylose isomerase activity comprises a specific activity at 37° C. in the range from about 1 to about 250 units per milligram of protein. Alternatively, xylose isomerase activity comprises a specific activity at 37° C. in the range from about 1 to about 100 units per milligram of protein.

In one aspect, the polypeptide comprises at least one glycosylation site, such as an N-linked glycosylation or an O-linked glycosylation. The polypeptide can be glycosylated after being expressed in a P. pastoris or a S. pombe.

In one aspect, the polypeptide can retain a xylose isomerase activity under conditions comprising about pH 6.5, pH 6, pH 5.5, pH 5, pH 4.5 or pH 4. In another aspect, the polypeptide can retain xylose isomerase activity under conditions comprising about pH 7, pH 7.5 pH 8.0, pH 8.5, pH 9, pH 9.5, pH 10, pH 10.5 or pH 11. In one aspect, the polypeptide can retain xylose isomerase activity after exposure to conditions comprising about pH 6.5, pH 6, pH 5.5, pH 5, pH 4.5 or pH 4. In another aspect, the polypeptide can retain xylose isomerase activity after exposure to conditions comprising about pH 7, pH 7.5 pH 8.0, pH 8.5, pH 9, pH 9.5, pH 10, pH 10.5 or pH 11.

The invention provides a protein preparation comprising a polypeptide of the invention, wherein the protein preparation comprises a liquid, a solid or a gel.

The invention provides a homodimer comprising a polypeptide of the invention. The invention provides a heterodimer comprising a polypeptide of the invention and a second domain. In one aspect, the second domain is a polypeptide and the heterodimer is a fusion protein. The second domain can be an epitope or a tag.

The invention provides an immobilized polypeptide having a xylose isomerase activity, wherein the polypeptide comprises a polypeptide of the invention, including antibodies, homodimers and heterodimers of the invention. The polypeptide can be immobilized on a cell, a metal, a resin, a polymer, a ceramic, a glass, a microelectrode, a graphitic particle, a bead, a gel, a plate, an array or a capillary tube.

The invention provides an array comprising an immobilized polypeptide or antibody of the invention. The invention provides an array comprising an immobilized nucleic acid of the invention.

The invention provides an isolated or recombinant antibody that specifically binds to a polypeptide of the invention or to a polypeptide encoded by a nucleic acid of the invention. The isolated or recombinant antibody can be a monoclonal or a polyclonal antibody.

The invention provides a hybridoma comprising an antibody that specifically binds to a polypeptide of the invention or to a polypeptide encoded by a nucleic acid of the invention.

The invention provides a food supplement for an animal comprising a polypeptide of the invention or a polypeptide encoded by a nucleic acid of the invention. In the food supplement the polypeptide can be glycosylated. The food supplement can comprise a glucose or a starch.

The invention provides an edible enzyme delivery matrix comprising a polypeptide of the invention or a polypeptide encoded by a nucleic acid of the invention, wherein the polypeptide comprises a xylose isomerase activity. The edible enzyme delivery matrix can comprise a glucose or a starch. The delivery matrix can be in any form, e.g., it can comprise a pellet, a tablet or an equivalent. In the edible enzyme delivery matrix polypeptide can be glycosylated or the xylose isomerase activity can be thermotolerant or thermostable.

The invention provides a method of isolating or identifying a polypeptide with a xylose isomerase activity comprising the steps of: (a) providing an antibody of the invention; (b) providing a sample comprising polypeptides; and (c) contacting the sample of step (b) with the antibody of step (a) under conditions wherein the antibody can specifically binds to the polypeptide, thereby isolating or identifying a polypeptide having a xylose isomerase activity.

The invention provides a method of making an anti-xylose isomerase antibody comprising administering to a non-human animal a nucleic acid of the invention, or a polypeptide of the invention, in an amount sufficient to generate a humoral immune response, thereby making an anti-xylose isomerase antibody.

The invention provides a method of producing a recombinant polypeptide comprising the steps of: (a) providing a nucleic acid operably of the invention linked to a promoter; and (b) expressing the nucleic acid of step (a) under conditions that allow expression of the polypeptide, thereby producing a recombinant polypeptide. The method can further comprise transforming a host cell with the nucleic acid of step (a) followed by expressing the nucleic acid of step (a), thereby producing a recombinant polypeptide in a transformed cell. The cell can be any cell, e.g., any plant cell.

The invention provides a method for identifying a polypeptide having a xylose isomerase activity comprising the following steps: (a) providing a polypeptide of the invention or a polypeptide encoded by a nucleic acid of the invention; (b) providing a xylose isomerase substrate; and (c) contacting the polypeptide or a fragment or variant thereof of step (a) with the substrate of step (b) and detecting a decrease in the amount of substrate or an increase in the amount of a reaction product, wherein a decrease in the amount of the substrate or an increase in the amount of the reaction product detects a polypeptide having a xylose isomerase activity. The substrate can be a glucose, a xylose, an α-D-glucopyranose, a β-D-glucopyranose and the like.

The invention provides a method for identifying a xylose isomerase substrate comprising the following steps: (a) providing a polypeptide of the invention or a polypeptide encoded by a nucleic acid of the invention; (b) providing a test substrate; and (c) contacting the polypeptide of step (a) with the test substrate of step (b) and detecting a decrease in the amount of substrate or an increase in the amount of reaction product, wherein a decrease in the amount of the substrate or an increase in the amount of a reaction product identifies the test substrate as a xylose isomerase substrate.

The invention provides a method of determining whether a test compound specifically binds to a polypeptide comprising the following steps: (a) expressing a nucleic acid or a vector comprising the nucleic acid under conditions permissive for translation of the nucleic acid to a polypeptide, wherein the nucleic acid has a sequence of the invention, or, providing a polypeptide of the invention; (b) providing a test compound; (c) contacting the polypeptide with the test compound; and (d) determining whether the test compound of step (b) specifically binds to the polypeptide.

The invention provides a method for identifying a modulator of a xylose isomerase activity comprising the following steps: (a) providing a polypeptide of the invention or a polypeptide encoded by a nucleic acid of the invention; (b) providing a test compound; (c) contacting the polypeptide of step (a) with the test compound of step (b) and measuring an activity of the xylose isomerase, wherein a change in the xylose isomerase activity measured in the presence of the test compound compared to the activity in the absence of the test compound provides a determination that the test compound modulates the xylose isomerase activity. In one aspect, the xylose isomerase activity is measured by providing a xylose isomerase substrate and detecting a decrease in the amount of the substrate or an increase in the amount of a reaction product, or, an increase in the amount of the substrate or a decrease in the amount of a reaction product. In one aspect, a decrease in the amount of the substrate or an increase in the amount of the reaction product with the test compound as compared to the amount of substrate or reaction product without the test compound identifies the test compound as an activator of xylose isomerase activity. In one aspect, an increase in the amount of the substrate or a decrease in the amount of the reaction product with the test compound as compared to the amount of substrate or reaction product without the test compound identifies the test compound as an inhibitor of xylose isomerase activity.

The invention provides a computer system comprising a processor and a data storage device wherein said data storage device has stored thereon a polypeptide sequence or a nucleic acid sequence, wherein the polypeptide sequence comprises sequence of the invention, or subsequence thereof, and the nucleic acid comprises a sequence of the invention, or subsequence thereof. The computer system can further comprise a sequence comparison algorithm and a data storage device having at least one reference sequence stored thereon. The sequence comparison algorithm can comprise a computer program that indicates polymorphisms. The computer system can further comprise an identifier that identifies one or more features in said sequence.

The invention provides a computer readable medium having stored thereon a polypeptide sequence or a nucleic acid sequence, wherein the polypeptide sequence comprises sequence of the invention, or subsequence thereof, and the nucleic acid comprises a sequence of the invention, or subsequence thereof.

The invention provides a method for identifying a feature in a sequence comprising the steps of: (a) reading the sequence using a computer program which identifies one or more features in a sequence, wherein the sequence comprises a polypeptide sequence or a nucleic acid sequence, wherein the polypeptide sequence comprises sequence of the invention or subsequence thereof, and the nucleic acid comprises a sequence of the invention or subsequence thereof; and (b) identifying one or more features in the sequence with the computer program.

The invention provides a method for comparing a first sequence to a second sequence comprising the steps of: (a) reading the first sequence and the second sequence through use of a computer program which compares sequences, wherein the first sequence comprises a polypeptide sequence or a nucleic acid sequence, wherein the polypeptide sequence comprises sequence of the invention, or subsequence thereof, and the nucleic acid comprises a sequence of the invention, or subsequence thereof; and (b) determining differences between the first sequence and the second sequence with the computer program. The step of determining differences between the first sequence and the second sequence can further comprise the step of identifying polymorphisms. The method can further comprise an identifier that identifies one or more features in a sequence. The method can comprise reading the first sequence using a computer program and identifying one or more features in the sequence.

The invention provides a method for isolating or recovering a nucleic acid encoding a polypeptide with a xylose isomerase activity from an environmental sample comprising the steps of: (a) providing an amplification primer sequence pair for amplifying a nucleic acid encoding a polypeptide with a xylose isomerase activity, wherein the primer pair is capable of amplifying a nucleic acid of the invention, e.g., SEQ ID NO:1 or SEQ ID NO:3, or a subsequence thereof; (b) isolating a nucleic acid from the environmental sample or treating the environmental sample such that nucleic acid in the sample is accessible for hybridization to the amplification primer pair; and, (c) combining the nucleic acid of step (b) with the amplification primer pair of step (a) and amplifying nucleic acid from the environmental sample, thereby isolating or recovering a nucleic acid encoding a polypeptide with a xylose isomerase activity from an environmental sample. In one aspect, each member of the amplification primer sequence pair comprises an oligonucleotide comprising at least about 10 to 50 consecutive bases of a sequence as set forth in SEQ ID NO:1, SEQ ID NO:3, or a subsequence thereof.

The invention provides a method for isolating or recovering a nucleic acid encoding a polypeptide with a xylose isomerase activity from an environmental sample comprising the steps of: (a) providing a polynucleotide probe comprising a sequence of the invention, or a subsequence thereof; (b) isolating a nucleic acid from the environmental sample or treating the environmental sample such that nucleic acid in the sample is accessible for hybridization to a polynucleotide probe of step (a); (c) combining the isolated nucleic acid or the treated environmental sample of step (b) with the polynucleotide probe of step (a); and (d) isolating a nucleic acid that specifically hybridizes with the polynucleotide probe of step (a), thereby isolating or recovering a nucleic acid encoding a polypeptide with a xylose isomerase activity from an environmental sample. In one aspect, the environmental sample comprises a water sample, a liquid sample, a soil sample, an air sample or a biological sample. The biological sample can be derived from a bacterial cell, a protozoan cell, an insect cell, a yeast cell, a plant cell, a fungal cell or a mammalian cell.

The invention provides a method of generating a variant of a nucleic acid encoding a polypeptide with a xylose isomerase activity comprising the steps of: (a) providing a template nucleic acid comprising a nucleic acid sequence of the invention; and (b) modifying, deleting or adding one or more nucleotides in the template sequence, or a combination thereof, to generate a variant of the template nucleic acid. The method can further comprise expressing the variant nucleic acid to generate a variant xylose isomerase polypeptide. The modifications, additions or deletions can be introduced by a method comprising error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturated mutagenesis (GSSM™), synthetic ligation reassembly (SLR) and a combination thereof. The modifications, additions or deletions can be introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof. The modifications, additions or deletions can be introduced by error-prone PCR. The modifications, additions or deletions can be introduced by shuffling. The modifications, additions or deletions can be introduced by oligonucleotide-directed mutagenesis. The modifications, additions or deletions can be introduced by assembly PCR. The modifications, additions or deletions can be introduced by sexual PCR mutagenesis. The modifications, additions or deletions can be introduced by in vivo mutagenesis. The modifications, additions or deletions can be introduced by cassette mutagenesis. The modifications, additions or deletions can be introduced by recursive ensemble mutagenesis. The modifications, additions or deletions can be introduced by exponential ensemble mutagenesis. The modifications, additions or deletions can be introduced by site-specific mutagenesis. The modifications, additions or deletions can be introduced by gene reassembly. The modifications, additions or deletions can be introduced by synthetic ligation reassembly (SLR). The modifications, additions or deletions can be introduced by gene site saturated mutagenesis (GSSM™).

In one aspect, the method is iteratively repeated until a xylose isomerase having an altered or different activity or an altered or different stability from that of a polypeptide encoded by the template nucleic acid is produced. The variant xylose isomerase polypeptide can be thermotolerant, and retains some activity after being exposed to an elevated temperature. The variant xylose isomerase polypeptide can have increased glycosylation as compared to the xylose isomerase encoded by a template nucleic acid. The variant xylose isomerase polypeptide can have a xylose isomerase activity under a high temperature, wherein the xylose isomerase encoded by the template nucleic acid is not active under the high temperature.

In one aspect, the method is iteratively repeated until a xylose isomerase coding sequence having an altered codon usage from that of the template nucleic acid is produced. In one aspect, the method is iteratively repeated until a xylose isomerase gene having higher or lower level of message expression or stability from that of the template nucleic acid is produced.

The invention provides a method for modifying codons in a nucleic acid encoding a polypeptide with a xylose isomerase activity to increase its expression in a host cell, the method comprising the following steps: (a) providing a nucleic acid encoding a polypeptide with a xylose isomerase activity comprising a nucleic acid of the invention, or a nucleic acid encoding the polypeptide of the invention; and, (b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon under-represented in coding sequences in genes in the host cell, thereby modifying the nucleic acid to increase its expression in a host cell.

The invention provides a method for modifying codons in a nucleic acid encoding a xylose isomerase polypeptide, the method comprising the following steps: (a) providing a nucleic acid encoding a polypeptide of the invention, or a nucleic acid encoding the polypeptide of the invention; and, (b) identifying a codon in the nucleic acid of step (a) and replacing it with a different codon encoding the same amino acid as the replaced codon, thereby modifying codons in a nucleic acid encoding a xylose isomerase.

The invention provides a method for modifying codons in a nucleic acid encoding a xylose isomerase polypeptide to increase its expression in a host cell, the method comprising the following steps: (a) providing a nucleic acid encoding a polypeptide of the invention, or a nucleic acid encoding a polypeptide of the invention; and, (b) identifying a non-preferred or a less preferred codon in the nucleic acid of step (a) and replacing it with a preferred or neutrally used codon encoding the same amino acid as the replaced codon, wherein a preferred codon is a codon over-represented in coding sequences in genes in the host cell and a non-preferred or less preferred codon is a codon under-represented in coding sequences in genes in the host cell, thereby modifying the nucleic acid to increase its expression in a host cell. The host cell can be a bacterial cell, a fungal cell, an insect cell, a yeast cell, a plant cell or a mammalian cell.

The invention provides a method for modifying a codon in a nucleic acid encoding a polypeptide having a xylose isomerase activity to decrease its expression in a host cell, the method comprising the following steps: (a) providing a nucleic acid encoding a polypeptide of the invention, or a nucleic acid encoding a polypeptide of the invention; and (b) identifying at least one preferred codon in the nucleic acid of step (a) and replacing it with a non-preferred or less preferred codon encoding the same amino acid as the replaced codon, wherein a preferred codon is a codon over-represented in coding sequences in genes in a host cell and a non-preferred or less preferred codon is a codon under-represented in coding sequences in genes in the host cell, thereby modifying the nucleic acid to decrease its expression in a host cell. The host cell can be a bacterial cell, a fungal cell, an insect cell, a yeast cell, a plant cell or a mammalian cell.

The invention provides a method for producing a library of nucleic acids encoding a plurality of modified xylose isomerase active sites or substrate binding sites, wherein the modified active sites or substrate binding sites are derived from a first nucleic acid comprising a sequence encoding a first active site or a first substrate binding site the method comprising the following steps: (a) providing a first nucleic acid encoding a first active site or first substrate binding site, wherein the first nucleic acid sequence comprises a sequence that hybridizes under stringent conditions to a nucleic acid of the invention, e.g., a sequence as set forth in SEQ ID NO:1, SEQ ID NO:3, or a subsequence thereof, or, a nucleic acid encoding a polypeptide of the invention, and the nucleic acid encodes a xylose isomerase active site or a xylose isomerase substrate binding site; (b) providing a set of mutagenic oligonucleotides that encode naturally-occurring amino acid variants at a plurality of targeted codons in the first nucleic acid; and, (c) using the set of mutagenic oligonucleotides to generate a set of active site-encoding or substrate binding site-encoding variant nucleic acids encoding a range of amino acid variations at each amino acid codon that was mutagenized, thereby producing a library of nucleic acids encoding a plurality of modified xylose isomerase active sites or substrate binding sites. In one aspect the method comprises mutagenizing the first nucleic acid of step (a) by a method comprising an optimized directed evolution system. In one aspect the method comprises mutagenizing the first nucleic acid of step (a) by a method comprising gene site-saturation mutagenesis (GSSM™). In one aspect the method comprises mutagenizing the first nucleic acid of step (a) by a method comprising a synthetic ligation reassembly (SLR). In one aspect the method comprises mutagenizing the first nucleic acid of step (a) or variants by a method comprising error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturated mutagenesis (GSSM™), synthetic ligation reassembly (SLR) and a combination thereof. In one aspect the method further comprises mutagenizing the first nucleic acid of step (a) or variants by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof.

The invention provides a method for making a small molecule comprising the following steps: (a) providing a plurality of biosynthetic enzymes capable of synthesizing or modifying a small molecule, wherein one of the enzymes comprises a xylose isomerase enzyme encoded by a nucleic acid of the invention; (b) providing a substrate for at least one of the enzymes of step (a); and (c) reacting the substrate of step (b) with the enzymes under conditions that facilitate a plurality of biocatalytic reactions to generate a small molecule by a series of biocatalytic reactions.

The invention provides a method for modifying a small molecule comprising the following steps: (a) providing a xylose isomerase enzyme, wherein the enzyme comprises a polypeptide of the invention, or, is encoded by a nucleic acid of the invention; (b) providing a small molecule; and (c) reacting the enzyme of step (a) with the small molecule of step (b) under conditions that facilitate an enzymatic reaction catalyzed by the xylose isomerase enzyme, thereby modifying a small molecule by a xylose isomerase enzymatic reaction. The method can comprise a plurality of small molecule substrates for the enzyme of step (a), thereby generating a library of modified small molecules produced by at least one enzymatic reaction catalyzed by the xylose isomerase enzyme. The method can further comprise a plurality of additional enzymes under conditions that facilitate a plurality of biocatalytic reactions by the enzymes to form a library of modified small molecules produced by the plurality of enzymatic reactions. The method can further comprise the step of testing the library to determine if a particular modified small molecule which exhibits a desired activity is present within the library. The method can comprise the step of testing the library further comprises the steps of systematically eliminating all but one of the biocatalytic reactions used to produce a portion of the plurality of the modified small molecules within the library by testing the portion of the modified small molecule for the presence or absence of the particular modified small molecule with a desired activity, and identifying at least one specific biocatalytic reaction that produces the particular modified small molecule of desired activity.

The invention provides a method for determining a functional fragment of a xylose isomerase enzyme comprising the steps of: (a) providing a xylose isomerase enzyme, wherein the enzyme comprises a polypeptide of the invention, or, is encoded by a nucleic acid of the invention; and (b) deleting a plurality of amino acid residues from the sequence of step (a) and testing the remaining subsequence for a xylose isomerase activity, thereby determining a functional fragment of a xylose isomerase enzyme. The xylose isomerase activity can be measured by providing a xylose isomerase substrate and detecting a decrease in the amount of the substrate or an increase in the amount of a reaction product.

The invention provides a method for whole cell engineering of new or modified phenotypes by using real-time metabolic flux analysis, the method comprising the following steps: (a) making a modified cell by modifying the genetic composition of a cell, wherein the genetic composition is modified by addition to the cell of a nucleic acid of the invention, or a nucleic acid encoding the polypeptide of the invention; (b) culturing the modified cell to generate a plurality of modified cells; (c) measuring at least one metabolic parameter of the cell by monitoring the cell culture of step (b) in real time; and, (d) analyzing the data of step (c) to determine if the measured parameter differs from a comparable measurement in an unmodified cell under similar conditions, thereby identifying an engineered phenotype in the cell using real-time metabolic flux analysis. The genetic composition of the cell can be modified by a method comprising deletion of a sequence or modification of a sequence in the cell, or, knocking out the expression of a gene. The method can further comprise selecting a cell comprising a newly engineered phenotype. The method can further comprise culturing the selected cell, thereby generating a new cell strain comprising a newly engineered phenotype.

The invention provides a method of increasing thermotolerance or thermostability of a xylose isomerase polypeptide, the method comprising glycosylating a xylose isomerase polypeptide, wherein the polypeptide comprises at least thirty contiguous amino acids of a sequence of the invention, or a polypeptide encoded by a nucleic acid of the invention, thereby increasing the thermotolerance or thermostability of the xylose isomerase polypeptide. The xylose isomerase specific activity can be thermostable or thermotolerant at a temperature in the range from greater than about 90° C. to about 130° C.

The invention provides a method for overexpressing a recombinant xylose isomerase polypeptide in a cell comprising expressing a vector comprising a nucleic acid comprising a nucleic acid sequence at least 96% sequence identity to the nucleic acid of claim 1 or claim 30 over a region of at least about 100 residues, wherein the sequence identities are determined by analysis with a sequence comparison algorithm or by visual inspection, wherein overexpression is effected by use of a high activity promoter, a dicistronic vector or by gene amplification of the vector.

The invention provides a kit comprising a polypeptide of the invention or a polypeptide encoded by a nucleic acid of the invention, wherein the polypeptide comprises a xylose isomerase activity.

The invention provides a method for catalyzing the isomerization of a glucose to a fructose comprising the following steps: (a) providing a polypeptide of the invention or a polypeptide encoded by a nucleic acid of the invention, wherein the polypeptide comprises a xylose isomerase activity; (b) providing a composition comprising a glucose; and (c) contacting the polypeptide of step (a) with the glucose of step (b) under conditions wherein the polypeptide of step (a) can isomerase the glucose to a fructose, thereby producing a fructose.

The invention provides a method for producing fructose from a starch comprising the following steps: (a) providing a polypeptide capable of hydrolyzing a α-1,4-glycosidic linkage in a starch; (b) contacting the polypeptide of the step (a) with the starch under condition wherein the polypeptide of step (a) can hydrolyze α-1,4-glycosidic linkages in the starch, thereby liquefying the starch to produce glucose; (c) providing a polypeptide of the invention or a polypeptide encoded by a nucleic acid of the invention, wherein the polypeptide comprises a xylose isomerase activity; and (d) contacting the polypeptide of step (c) with the glucose of step (b) under conditions wherein the polypeptide of step (c) can isomerase glucose, thereby producing fructose. The polypeptide of step (a) can comprise an xylose isomerase or a glucoamylase. The polypeptide can be capable of hydrolyzing α-1,6-glycosidic linkage in a starch.

The invention provides a method for producing fructose comprising the following steps: (a) providing a composition comprising a glucose; (b) providing a polypeptide having a xylose isomerase activity, wherein the polypeptide comprises an amino acid sequence of the invention, or, a polypeptide encoded by a nucleic acid of the invention; and (c) contacting the polypeptide of step (b) with the glucose of step (a) under conditions wherein the polypeptide can isomerase glucose thereby producing fructose. The conditions can comprise a temperature of between about 70° C. and 95° C., thereby shifting equilibrium of the reaction towards formation of fructose. The conditions can comprise a temperature of between about 80° C. and 90° C., thereby shifting equilibrium of the reaction towards formation of fructose. The polypeptide can be immobilized.

The invention provides a method of making fructose in a feed or a food prior comprising the following steps: (a) obtaining a feed or a food material comprising a starch, (b) providing a polypeptide capable of hydrolyzing a α-1,4-glycosidic linkage in a starch; (c) contacting the polypeptide of the step (a) with the feed or a food material under conditions wherein the polypeptides of step (a) can hydrolyze α-1,4-glycosidic linkages in the starch to produce a glucose; (d) providing a polypeptide of the invention or a polypeptide encoded by a nucleic acid of the invention, wherein the polypeptide comprises a xylose isomerase activity; and (e) adding the polypeptide of step (d) to the feed or food material in an amount sufficient to cause isomerization of the glucose to a fructose in the food or the feed. The food or feed can comprise rice, corn, barley, wheat, legumes, or potato. The polypeptide can be capable of hydrolyzing α-1,6-glycosidic linkage in a starch.

The invention provides a method for producing a high-fructose syrup comprising the following steps: (a) providing a polypeptide capable of hydrolyzing α-1,4-glycosidic linkages in a starch; (b) providing a composition comprising a starch; (c) contacting the polypeptides of step (a) and the composition of step (b) under conditions wherein the polypeptide of step (a) can hydrolyze α-1,4-glycosidic linkages in the starch; (d) providing a polypeptide of the invention or a polypeptide encoded by a nucleic acid of the invention, wherein the polypeptide comprises a xylose isomerase activity; and (e) contacting the polypeptide of step (d) and the starch hydrolysate of step (c) under conditions wherein the polypeptide of step (d) can isomerase glucose in the starch hydrolysate to a fructose, thereby producing the high-fructose syrup. The composition can comprise a rice, a corn, a barley, a wheat, a legume, a potato or a sweet potato. The composition can comprise a rice and the high-fructose syrup is a high-fructose corn syrup. The polypeptide can be capable of hydrolyzing α-1,6-glycosidic linkage in a starch. In one aspect, all reactions are carried out in one vessel. The high-fructose syrup can comprise an insecticide bait composition.

The invention provides a method for producing a high-fructose syrup comprising the following steps: (a) providing a transgenic seed or grain comprising a polypeptide of the invention or a polypeptide encoded by a nucleic acid of the invention, comprising a xylose isomerase activity, wherein the seed or grain comprises a starch; (b) expressing the xylose isomerase in the seed or grain; (c) hydrolyzing the starch to a glucose under conditions wherein the polypeptide of step (a) expressed in the seed or grain can catalyze isomerization of glucose to a fructose, thereby producing the high-fructose syrup. The steps of hydrolyzing the starch and isomerizing the glucose can be carried out at pH 4.0 to 6.5 and at temperature comprising a range of about 55° C. to 105° C.

The invention provides a method for producing fructose in brewing or alcohol production comprising the following steps: (a) providing a polypeptide of the invention or a polypeptide encoded by a nucleic acid of the invention, wherein the polypeptide comprises a xylose isomerase activity; (b) providing malt or mash composition comprising a glucose; and (c) contacting the polypeptide of step (a) with the composition of step (b) under conditions wherein the polypeptide of step (a) isomerizes the glucose of step (b) to a fructose, thereby producing fructose for brewing or alcohol production.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

The following drawings are illustrative of embodiments of the invention and are not meant to limit the scope of the invention as encompassed by the claims.

FIG. 1 is a block diagram of a computer system.

FIG. 2 is a flow diagram illustrating one aspect of a process for comparing a new nucleotide or protein sequence with a database of sequences in order to determine the homology levels between the new sequence and the sequences in the database.

FIG. 3 is a flow diagram illustrating one aspect of a process in a computer for determining whether two sequences are homologous.

FIG. 4 is a flow diagram illustrating one aspect of an identifier process 300 for detecting the presence of a feature in a sequence.

FIG. 5 illustrates an exemplary method to test for xylose isomerase activity, as described in Example 2, below.

FIG. 6 illustrates the results of tests for xylose isomerase activity for the exemplary enzymes having a sequence as set forth in SEQ ID NO:2 and SEQ ID NO:4, as described in Example 3, below: for SEQ ID NO:2, Absorbance (Ab) at 540 nm over time in minutes is summarized in the graph of FIG. 6A and Relative Activity as a function of pH is summarized in the graph of FIG. 6B; for SEQ ID NO:4, Absorbance (Ab) at 540 nm over time in minutes is summarized in the graph of FIG. 6C and Relative Activity as a function of pH is summarized in the graph of FIG. 6D.

FIG. 7 illustrates the results of tests for xylose isomerase activity for the exemplary enzyme having a sequence as set forth in SEQ ID NO:2 and SEQ ID NO:4, as described in Example 3, below: for the exemplary protein having a sequence as set forth in SEQ ID NO:2: Absorbance (Ab) at 540 nm over time in minutes at various temperatures as indicated is summarized in the graph of FIG. 7A and Relative Activity as a function of temperature is summarized in the graph of FIG. 7B. For the exemplary protein having a sequence as set forth in SEQ ID NO:4: Absorbance (Ab) at 540 nm over time in minutes at various temperatures as indicated is summarized in the graph of FIG. 7C and Relative Activity as a function of temperature is summarized in the graph of FIG. 7D.

FIG. 8 illustrates the results of tests for xylose isomerase activity for the exemplary enzyme having a sequence as set forth in SEQ ID NO:2 and SEQ ID NO:4, as described in Example 3, below: for the exemplary protein having a sequence as set forth in SEQ ID NO:2: Absorbance (Ab) at 540 nm over time in minutes at various time points as indicated is summarized in the graph of FIG. 8A and Relative Activity as a function of incubation time is summarized in the graph of FIG. 8B. For the exemplary protein having a sequence as set forth in SEQ ID NO:4: Absorbance (Ab) at 540 nm over time in minutes at various time points as indicated is summarized in the graph of FIG. 8C and Relative Activity as a function of time is summarized in the graph of FIG. 8D.

FIG. 9 illustrates the results of tests for xylose isomerase activity for the exemplary enzyme having a sequence as set forth in SEQ ID NO:2 and SEQ ID NO:4, as described in Example 3, below: for the exemplary protein having a sequence as set forth in SEQ ID NO:2: relative activity at various concentrations of Co and Mg as indicated is summarized in the graph of FIG. 9A. For the exemplary protein having a sequence as set forth in SEQ ID NO:4: relative activity at various concentrations of Co and Mg as indicated is summarized in the graph of FIG. 9B.

Like reference symbols in the various drawings indicate like elements.

### DETAILED DESCRIPTION

The invention provides polypeptides and peptides having xylose isomerase (also called glucose isomerase) activity, antibodies that bind to them, polynucleotides encoding the enzymes, methods of making and using these polynucleotides and polypeptides. The polypeptides and peptides of the invention can be used in a variety of agricultural and industrial contexts. In alternative aspects, a xylose isomerase activity of a polypeptide or peptide of the invention comprises: isomerization of xylose to xylulose; isomerization of glucose to fructose; isomerization of a D-glucose to a D-fructose; catalysis of the conversion of D-xylose to an equilibrium mixture of D-xylulose and D-xylose; isomerization of β-D-glucopyranose to β-D-fructopyranose; and/or, isomerization of α-D-glucopyranose to α-D-fructofuranose, or, isomerization of xylulose to xylose; isomerization of fructose to glucose; isomerization of a D-fructose to D-glucose; catalysis of the conversion of an equilibrium mixture of D-xylulose and D-xylose to D-xylose; isomerization of β-D-fructopyranose to β-D-glucopyranose; and/or, isomerization of α-D-fructofuranose to α-D-glucopyranose.

The polypeptides or peptides of the invention can be used for manufacturing high content fructose syrups, e.g., corn syrups. These processes can manufacture high-fructose compositions in large quantities. The polypeptides or peptides of the invention can be used in liquefied starch manufacturing processes if one of the end products desired is a fructose. The polypeptides or peptides of the invention can be used in starch hydrolysis processes if one of the end products desired is a fructose. The polypeptides or peptides of the invention can be used in food or animal feed manufacturing processes. Additionally, the polypeptides or peptides of the invention can be used in confectionary, brewing, alcohol and soft drinks production, and in diabetic foods and sweeteners.

In one aspect, the xylose isomerases of the invention are active at a high and/or at a low temperature, or, over a wide range of temperatures, e.g., they can be active in the temperatures ranging between about 1° C. to 30° C., or, between about 30° C. to 60° C., or, between about 60° C. to 130° C., between about 70° C. to 105° C., between about 80° C. to 95° C., between about 85° C. to 90° C., between about 100° C. to 130° C. In one aspect, these reactions are run at elevated temperatures to push the equilibrium of the reaction toward the reaction product, e.g., xylulose, fructose (such as D-fructose), a mixture of D-xylulose and D-xylose, β-D-fructopyranose, and/or, α-D-fructofuranose, e.g., between about 80° C. to 95° C., between about 85° C. to 90° C. and the like.

In one aspect, the xylose isomerases of the invention are active under conditions of low water activity (low water content). In one aspect, the xylose isomerases of the invention are active under conditions of low water content in the temperature range of between about 60° C. to about 120° C., or, between about 100° C. to 130° C.

The invention also provides xylose isomerases that have activity at neutral to alkaline pHs or at acidic to neutral pHs. In alternative aspects, the xylose isomerases of the invention can have activity in acidic pHs of about pH 6.5, pH 6.0, pH 5.5, pH 5.0, pH 4.5, and pH 4.0 or more acidic. In alternative aspects, the xylose isomerases of the invention can have activity in neutral to alkaline pHs of about pH 8.0, pH 8.5, pH 9.0, pH 9.5, pH 10, pH 0.5 or pH 11 or more alkaline.

The invention also provides methods for further modifying the exemplary xylose isomerases of the invention to generate proteins with alternative, e.g., different or new, properties. For example, xylose isomerases generated by the methods of the invention can have altered enzymatic activity, thermal stability, pH/activity profile, pH/stability profile (such as increased stability at low, e.g. pH<6 or pH<5, or high, e.g. pH>9, pH values), stability towards oxidation, Ca²⁺ or Mn²⁺ dependency, specific activity and the like. The invention provides methods for altering or adding any property of interest, e.g., an activity, a substrate, a temperature or pH optimum and the like. For instance, an alteration can result in a variant which, as compared to a parent enzyme, has altered enzymatic activity, or, pH or temperature activity profiles.

Definitions

The term “xylose isomerase” includes polypeptides, peptides, antibodies, enzymes having, e.g., a D-xylose isomerase activity, for example, enzymes which catalyze conversion of D-xylose to D-xylulose and glucose to fructose. A xylose isomerase activity of a polypeptide, peptide, antibody of the invention can comprise isomerization of xylose to xylulose; isomerization of glucose to fructose; isomerization of a D-glucose to a D-fructose; catalysis of the conversion of D-xylose to an equilibrium mixture of D-xylulose and D-xylose; isomerization of β-D-glucopyranose to β-D-fructopyranose; and/or, isomerization of α-D-glucopyranose to α-D-fructofuranose, or, isomerization of xylulose to xylose; isomerization of fructose to glucose; isomerization of a D-fructose to D-glucose; catalysis of the conversion of an equilibrium mixture of D-xylulose and D-xylose to D-xylose; isomerization of β-D-fructopyranose to β-D-glucopyranose; and/or, isomerization of α-D-fructofuranose to α-D-glucopyranose. The term also includes xylose isomerases capable of isomerizing bonds at high temperatures, low temperatures, alkaline pHs and at acidic pHs.

A “xylose isomerases variant” can have an amino acid sequence which is derived from the amino acid sequence of a “precursor xylose isomerases”. The precursor xylose isomerases include naturally-occurring xylose isomerases and recombinant xylose isomerases. The amino acid sequence of the xylose isomerases variant is “derived” from the precursor xylose isomerases amino acid sequence by the substitution, deletion or insertion of one or more amino acids of the precursor amino acid sequence. Such modification is of the “precursor DNA sequence” which encodes the amino acid sequence of the precursor xylose isomerases rather than manipulation of the precursor xylose isomerases enzyme per se. Suitable methods for such manipulation of the precursor DNA sequence include methods disclosed herein, as well as methods known to those skilled in the art.

The term “antibody” includes a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

The terms “array” or “microarray” or “biochip” or “chip” as used herein is a plurality of target elements, each target element comprising a defined amount of one or more polypeptides (including antibodies) or nucleic acids immobilized onto a defined area of a substrate surface, as discussed in further detail, below.

As used herein, the terms “computer,” “computer program” and “processor” are used in their broadest general contexts and incorporate all such devices, as described in detail, below.

A “coding sequence of” or a “sequence encodes” a particular polypeptide or protein, is a nucleic acid sequence which is transcribed and translated into a polypeptide or protein when placed under the control of appropriate regulatory sequences.

The term “expression cassette” as used herein refers to a nucleotide sequence which is capable of affecting expression of a structural gene (i.e., a protein coding sequence, such as a xylose isomerase of the invention) in a host compatible with such sequences. Expression cassettes include at least a promoter operably linked with the polypeptide coding sequence; and, optionally, with other sequences, e.g., transcription termination signals. Additional factors necessary or helpful in effecting expression may also be used, e.g., enhancers. “Operably linked” as used herein refers to linkage of a promoter upstream from a DNA sequence such that the promoter mediates transcription of the DNA sequence. Thus, expression cassettes also include plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and includes both the expression and non-expression plasmids. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

The term “gene” can include a nucleic acid sequence comprising a segment of DNA involved in producing a transcription product (e.g., a message), which in turn is translated to produce a polypeptide chain, or regulates gene transcription, reproduction or stability. Genes can include, inter alia, regions preceding and following the coding region, such as leader and trailer, promoters and enhancers, as well as, where applicable, intervening sequences (introns) between individual coding segments (exons).

The phrases “nucleic acid” or “nucleic acid sequence” can include an oligonucleotide, nucleotide, polynucleotide, or to a fragment of any of these, to DNA or RNA (e.g., mRNA, rRNA, tRNA) of genomic or synthetic origin which may be single-stranded or double-stranded and may represent a sense or antisense strand, to peptide nucleic acid (PNA), or to any DNA-like or RNA-like material, natural or synthetic in origin, including, e.g., iRNA, ribonucleoproteins (e.g., iRNPs). The term encompasses nucleic acids, i.e., oligonucleotides, containing known analogues of natural nucleotides. The term also encompasses nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156.

As used herein, the term “promoter” includes all sequences capable of driving transcription of a coding sequence in a cell, e.g., a plant cell. Thus, promoters used in the constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription. “Constitutive” promoters are those that drive expression continuously under most environmental conditions and states of development or cell differentiation. “Inducible” or “regulatable” promoters direct expression of the nucleic acid of the invention under the influence of environmental conditions or developmental conditions. Examples of environmental conditions that may affect transcription by inducible promoters include anaerobic conditions, elevated temperature, drought, or the presence of light.

“Tissue-specific” promoters are transcriptional control elements that are only active in particular cells or tissues or organs, e.g., in plants or animals. Tissue-specific regulation may be achieved by certain intrinsic factors which ensure that genes encoding proteins specific to a given tissue are expressed. Such factors are known to exist in mammals and plants so as to allow for specific tissues to develop.

The term “plant” includes whole plants, plant parts (e.g., leaves, stems, flowers, roots, etc.), plant protoplasts, seeds and plant cells and progeny of same. The class of plants which can be used in the method of the invention is generally as broad as the class of higher plants amenable to transformation techniques, including angiosperms (monocotyledonous and dicotyledonous plants), as well as gymnosperms. It includes plants of a variety of ploidy levels, including polyploid, diploid, haploid and hemizygous states. As used herein, the term “transgenic plant” includes plants or plant cells into which a heterologous nucleic acid sequence has been inserted, e.g., the nucleic acids and various recombinant constructs (e.g., expression cassettes) of the invention.

“Amino acid” or “amino acid sequence” can include an oligopeptide, peptide, polypeptide, or protein sequence, or to a fragment, portion, or subunit of any of these, and to naturally occurring or synthetic molecules.

The terms “polypeptide” and “protein” can include amino acids joined to each other by peptide bonds or modified peptide bonds, i.e., peptide isosteres, and may contain modified amino acids other than the 20 gene-encoded amino acids. The term “polypeptide” also includes peptides and polypeptide fragments, motifs and the like. The term also includes glycosylated polypeptides. The peptides and polypeptides of the invention also include all “mimetic” and “peptidomimetic” forms, as described in further detail, below.

The term “isolated” can mean that the material is removed from its original environment (e.g., the natural environment if it is naturally occurring). For example, a naturally occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. As used herein, an isolated material or composition can also be a “purified” composition, i.e., it does not require absolute purity; rather, it is intended as a relative definition. Individual nucleic acids obtained from a library can be conventionally purified to electrophoretic homogeneity. In alternative aspects, the invention provides nucleic acids which have been purified from genomic DNA or from other sequences in a library or other environment by at least one, two, three, four, five or more orders of magnitude.

The term “recombinant” can mean that the nucleic acid is adjacent to a “backbone” nucleic acid to which it is not adjacent in its natural environment. In one aspect, nucleic acids represent 5% or more of the number of nucleic acid inserts in a population of nucleic acid “backbone molecules.” “Backbone molecules” according to the invention include nucleic acids such as expression vectors, self-replicating nucleic acids, viruses, integrating nucleic acids, and other vectors or nucleic acids used to maintain or manipulate a nucleic acid insert of interest. In one aspect, the enriched nucleic acids represent 15%, 20%, 30%, 40%, 50%, 60%, 70%, 80%, 90% or more of the number of nucleic acid inserts in the population of recombinant backbone molecules. “Recombinant” polypeptides or proteins refer to polypeptides or proteins produced by recombinant DNA techniques; e.g., produced from cells transformed by an exogenous DNA construct encoding the desired polypeptide or protein. “Synthetic” polypeptides or protein are those prepared by chemical synthesis, as described in further detail, below.

A promoter sequence can be “operably linked to” a coding sequence when RNA polymerase which initiates transcription at the promoter will transcribe the coding sequence into mRNA, as discussed further, below.

“Oligonucleotide” can include either a single stranded polydeoxynucleotide or two complementary polydeoxynucleotide strands which may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

The phrase “substantially identical” in the context of two nucleic acids or polypeptides, can refer to two or more sequences that have, e.g., at least about 50%, 55%, 60%, 65%, 70%, 75%, 80%, 85%, 90%, 95%, 98% or 99% or more nucleotide or amino acid residue (sequence) identity, when compared and aligned for maximum correspondence, as measured using one any known sequence comparison algorithm, as discussed in detail below, or by visual inspection. In alternative aspects, the invention provides nucleic acid and polypeptide sequences having substantial identity to an exemplary sequence of the invention, e.g., SEQ ID NO:1, or SEQ ID NO:3, over a region of at least about 10, 20, 30, 40, 50, 100, 150, 200, 250, 300, 350, 400, 450, 500, 550, 600, 650, 700, 750, 800, 850, 900, 950, 1000 residues, or a region ranging from between about 50 residues to the full length of the nucleic acid or polypeptide. Nucleic acid sequences of the invention can be substantially identical over the entire length of a polypeptide coding region.

Additionally a “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from a xylose isomerase, resulting in modification of the structure of the polypeptide, without significantly altering its biological activity. For example, amino- or carboxyl-terminal amino acids that are not required for xylose isomerase activity can be removed.

“Hybridization” refers to the process by which a nucleic acid strand joins with a complementary strand through base pairing. Hybridization reactions can be sensitive and selective so that a particular sequence of interest can be identified even in samples in which it is present at low concentrations. Stringent conditions can be defined by, for example, the concentrations of salt or formamide in the prehybridization and hybridization solutions, or by the hybridization temperature, and are well known in the art. For example, stringency can be increased by reducing the concentration of salt, increasing the concentration of formamide, or raising the hybridization temperature, altering the time of hybridization, as described in detail, below. In alternative aspects, nucleic acids of the invention are defined by their ability to hybridize under various stringency conditions (e.g., high, medium, and low), as set forth herein.

The term “variant” can include polynucleotides or polypeptides of the invention modified at one or more base pairs, codons, introns, exons, or amino acid residues (respectively) yet still retain the biological activity of a xylose isomerase of the invention. Variants can be produced by any number of means included methods such as, for example, error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturated mutagenesis (GSSM™) and any combination thereof. Techniques for producing variant xylose isomerases having activity at a pH or temperature, for example, that is different from a wild-type xylose isomerase, are included herein.

The term “gene site saturated mutagenesis” or “GSSM™” includes a method that uses degenerate oligonucleotide primers to introduce point mutations into a polynucleotide, as described in detail, below. The term “optimized directed evolution system” or “optimized directed evolution” includes a method for reassembling fragments of related nucleic acid sequences, e.g., related genes, and explained in detail, below. The term “synthetic ligation reassembly” or “SLR” includes a method of ligating oligonucleotide fragments in a non-stochastic fashion, and explained in detail, below.

The term “syrup” can be defined as an aqueous solution or slurry comprising carbohydrates such as mono-, oligo- or polysaccharides.

Generating and Manipulating Nucleic Acids

The invention provides nucleic acids, including expression cassettes such as expression vectors, encoding the polypeptides and peptides (e.g., xylose isomerases, antibodies) of the invention. The invention also includes methods for discovering new xylose isomerase sequences using the nucleic acids of the invention. Also provided are methods for modifying the nucleic acids of the invention by, e.g., synthetic ligation reassembly, optimized directed evolution system and/or saturation mutagenesis.

The nucleic acids of the invention can be made, isolated and/or manipulated by, e.g., cloning and expression of cDNA libraries, amplification of message or genomic DNA by PCR, and the like. In practicing the methods of the invention, homologous genes can be modified by manipulating a template nucleic acid, as described herein. The invention can be practiced in conjunction with any method or protocol or device known in the art, which are well described in the scientific and patent literature.

General Techniques

The nucleic acids used to practice this invention, whether RNA, iRNA, antisense nucleic acid, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Left. 22:1859; U.S. Pat. No. 4,458,066.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

In one aspect, a nucleic acid encoding a polypeptide of the invention is assembled in appropriate phase with a leader sequence capable of directing secretion of the translated polypeptide or fragment thereof.

The invention provides fusion proteins and nucleic acids encoding them. A polypeptide of the invention can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-414). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll (1993) DNA Cell. Biol., 12:441-53.

Transcriptional and Translational Control Sequences

The invention provides nucleic acid (e.g., DNA) sequences of the invention operatively linked to expression (e.g., transcriptional or translational) control sequence(s), e.g., promoters or enhancers, to direct or modulate RNA synthesis/expression. The expression control sequence can be in an expression vector. Exemplary bacterial promoters include lacI, lacZ, T3, T7, gpt, lambda PR, PL and trp. Exemplary eukaryotic promoters include CMV immediate early, HSV thymidine kinase, early and late SV40, LTRs from retrovirus, and mouse metallothionein I.

Promoters suitable for expressing a polypeptide in bacteria include the E. coli lac or trp promoters, the lacI promoter, the lacZ promoter, the T3 promoter, the T7 promoter, the gpt promoter, the lambda PR promoter, the lambda PL promoter, promoters from operons encoding glycolytic enzymes such as 3-phosphoglycerate kinase (PGK), and the acid phosphatase promoter. Eukaryotic promoters include the CMV immediate early promoter, the HSV thymidine kinase promoter, heat shock promoters, the early and late SV40 promoter, LTRs from retroviruses, and the mouse metallothionein-I promoter. Other promoters known to control expression of genes in prokaryotic or eukaryotic cells or their viruses may also be used.

Tissue-Specific Plant Promoters

The invention provides expression cassettes that can be expressed in a tissue-specific manner, e.g., that can express a xylose isomerase of the invention in a tissue-specific manner. The invention also provides plants or seeds that express a xylose isomerase of the invention in a tissue-specific manner. The tissue-specificity can be seed specific, stem specific, leaf specific, root specific, fruit specific and the like.

In one aspect, a constitutive promoter such as the CaMV 35S promoter can be used for expression in specific parts of the plant or seed or throughout the plant. For example, for overexpression of a xylose isomerase of the invention, a plant promoter fragment can be employed which will direct expression of a nucleic acid in some or all tissues of a plant, e.g., a regenerated plant. Such “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumefaciens, and other transcription initiation regions from various plant genes known to those of skill. Such genes include, e.g., ACT11 from Arabidopsis (Huang (1996) Plant Mol. Biol. 33:125-139); Cat3 from Arabidopsis (GenBank No. U43147, Zhong (1996) Mol. Gen. Genet. 251:196-203); the gene encoding stearoyl-acyl carrier protein desaturase from Brassica napus (Genbank No. X74782, Solocombe (1994) Plant Physiol. 104:1167-1176); GPc1 from maize (GenBank No. X15596; Martinez (1989) J. Mol. Biol. 208:551-565); the Gpc2 from maize (GenBank No. U45855, Manjunath (1997) Plant Mol. Biol. 33:97-112); plant promoters described in U.S. Pat. Nos. 4,962,028; 5,633,440.

The invention uses tissue-specific or constitutive promoters derived from viruses which can include, e.g., the tobamovirus subgenomic promoter (Kumagai (1995) Proc. Natl. Acad. Sci. USA 92:1679-1683; the rice tungro bacilliform virus (RTBV), which replicates only in phloem cells in infected rice plants, with its promoter which drives strong phloem-specific reporter gene expression; the cassaya vein mosaic virus (CVMV) promoter, with highest activity in vascular elements, in leaf mesophyll cells, and in root tips (Verdaguer (1996) Plant Mol. Biol. 31:1129-1139).

Alternatively, the plant promoter may direct expression of a xylose isomerase-expressing nucleic acid in a specific tissue, organ or cell type (i.e. tissue-specific promoters) or may be otherwise under more precise environmental or developmental control or under the control of an inducible promoter. Examples of environmental conditions that may affect transcription include anaerobic conditions, elevated temperature, the presence of light, or sprayed with chemicals/hormones. For example, the invention incorporates the drought-inducible promoter of maize (Busk (1997) supra); the cold, drought, and high salt inducible promoter from potato (Kirch (1997) Plant Mol. Biol. 33:897 909).

Tissue-specific promoters can promote transcription only within a certain time frame of developmental stage within that tissue. See, e.g., Blazquez (1998) Plant Cell 10:791-800, characterizing the Arabidopsis LEAFY gene promoter. See also Cardon (1997) Plant J 12:367-77, describing the transcription factor SPL3, which recognizes a conserved sequence motif in the promoter region of the A. thaliana floral meristem identity gene AP1; and Mandel (1995) Plant Molecular Biology, Vol. 29, pp 995-1004, describing the meristem promoter eIF4. Tissue specific promoters which are active throughout the life cycle of a particular tissue can be used. In one aspect, the nucleic acids of the invention are operably linked to a promoter active primarily only in cotton fiber cells. In one aspect, the nucleic acids of the invention are operably linked to a promoter active primarily during the stages of cotton fiber cell elongation, e.g., as described by Rinehart (1996) supra. The nucleic acids can be operably linked to the Fbl2A gene promoter that can be expressed in cotton fiber cells (Ibid). See also, John (1997) Proc. Natl. Acad. Sci. USA 89:5769-5773; John, et al., U.S. Pat. Nos. 5,608,148 and 5,602,321, describing cotton fiber-specific promoters and methods for the construction of transgenic cotton plants. Root-specific promoters may also be used to express the nucleic acids of the invention. Examples of root-specific promoters include the promoter from the alcohol dehydrogenase gene (DeLisle (1990) Int. Rev. Cytol. 123:39-60). Other promoters that can be used to express the nucleic acids of the invention include, e.g., ovule-specific, embryo-specific, endosperm-specific, integument-specific, seed coat-specific promoters, or some combination thereof; a leaf-specific promoter (see, e.g., Busk (1997) Plant J. 11:1285 1295, describing a leaf-specific promoter in maize); the ORF13 promoter from Agrobacterium rhizogenes (which exhibits high activity in roots, see, e.g., Hansen (1997) supra); a maize pollen specific promoter (see, e.g., Guerrero (1990) Mol. Gen. Genet. 224:161 168); a tomato promoter active during fruit ripening, senescence and abscission of leaves and, to a lesser extent, of flowers can be used (see, e.g., Blume (1997) Plant J. 12:731 746); a pistil-specific promoter from the potato SK2 gene (see, e.g., Ficker (1997) Plant Mol. Biol. 35:425 431); the Blec4 gene from pea, which is active in epidermal tissue of vegetative and floral shoot apices of transgenic alfalfa making it a useful tool to target the expression of foreign genes to the epidermal layer of actively growing shoots or fibers; the ovule-specific BEL1 gene (see, e.g., Reiser (1995) Cell 83:735-742, GenBank No. U39944); and/or, the promoter in Klee, U.S. Pat. No. 5,589,583, describing a plant promoter region is capable of conferring high levels of transcription in meristematic tissue and/or rapidly dividing cells.

Alternatively, plant promoters which are inducible upon exposure to plant hormones, such as auxins, are used to express the nucleic acids of the invention. For example, the invention can use the auxin-response elements E1 promoter fragment (AuxREs) in the soybean (Glycine max L.) (Liu (1997) Plant Physiol. 115:397-407); the auxin-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37:906-913); a plant biotin response element (Streit (1997) Mol. Plant Microbe Interact. 10:933-937); and, the promoter responsive to the stress hormone abscisic acid (Sheen (1996) Science 274:1900-1902).

The nucleic acids of the invention can also be operably linked to plant promoters which are inducible upon exposure to chemicals reagents which can be applied to the plant, such as herbicides or antibiotics. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. Coding sequence can be under the control of, e.g., a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324). Using chemically- (e.g., hormone- or pesticide-) induced promoters, i.e., promoter responsive to a chemical which can be applied to the transgenic plant in the field, expression of a polypeptide of the invention can be induced at a particular stage of development of the plant. Thus, the invention also provides for transgenic plants containing an inducible gene encoding for polypeptides of the invention whose host range is limited to target plant species, such as corn, rice, barley, wheat, potato or other crops, inducible at any stage of development of the crop.

One of skill will recognize that a tissue-specific plant promoter may drive expression of operably linked sequences in tissues other than the target tissue. Thus, a tissue-specific promoter is one that drives expression preferentially in the target tissue or cell type, but may also lead to some expression in other tissues as well.

The nucleic acids of the invention can also be operably linked to plant promoters which are inducible upon exposure to chemicals reagents. These reagents include, e.g., herbicides, synthetic auxins, or antibiotics which can be applied, e.g., sprayed, onto transgenic plants. Inducible expression of the amylase-producing nucleic acids of the invention will allow the grower to select plants with the optimal starch/sugar ratio. The development of plant parts can thus controlled. In this way the invention provides the means to facilitate the harvesting of plants and plant parts. For example, in various embodiments, the maize In2-2 promoter, activated by benzenesulfonamide herbicide safeners, is used (De Veylder (1997) Plant Cell Physiol. 38:568-577); application of different herbicide safeners induces distinct gene expression patterns, including expression in the root, hydathodes, and the shoot apical meristem. Coding sequences of the invention are also under the control of a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324).

If proper polypeptide expression is desired, a polyadenylation region at the 3′-end of the coding region should be included. The polyadenylation region can be derived from the natural gene, from a variety of other plant genes, or from genes in the Agrobacterial T-DNA.

Expression Vectors and Cloning Vehicles

The invention provides expression vectors and cloning vehicles comprising nucleic acids of the invention, e.g., sequences encoding the xylose isomerases of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors of the invention can include chromosomal, non-chromosomal and synthetic DNA sequences. Large numbers of suitable vectors are known to those of skill in the art, and are commercially available. Exemplary vectors are include: bacterial: pQE vectors (Qiagen), pBluescript plasmids, pNH vectors, (lambda-ZAP vectors (Stratagene); ptrc99a, pKK223-3, pDR540, pRIT2T (Pharmacia); Eukaryotic: pXT1, pSG5 (Stratagene), pSVK3, pBPV, pMSG, pSVLSV40 (Pharmacia). However, any other plasmid or other vector may be used so long as they are replicable and viable in the host. Low copy number or high copy number vectors may be employed with the present invention.

The expression vector may comprise a promoter, a ribosome binding site for translation initiation and a transcription terminator. The vector may also include appropriate sequences for amplifying expression. Mammalian expression vectors can comprise an origin of replication, any necessary ribosome binding sites, a polyadenylation site, splice donor and acceptor sites, transcriptional termination sequences, and 5′ flanking non-transcribed sequences. In some aspects, DNA sequences derived from the SV40 splice and polyadenylation sites may be used to provide the required non-transcribed genetic elements.

In one aspect, the expression vectors contain one or more selectable marker genes to permit selection of host cells containing the vector. Such selectable markers include genes encoding dihydrofolate reductase or genes conferring neomycin resistance for eukaryotic cell culture, genes conferring tetracycline or ampicillin resistance in E. coli, and the S. cerevisiae TRP1 gene. Promoter regions can be selected from any desired gene using chloramphenicol transferase (CAT) vectors or other vectors with selectable markers.

Vectors for expressing the polypeptide or fragment thereof in eukaryotic cells may also contain enhancers to increase expression levels. Enhancers are cis-acting elements of DNA, usually from about 10 to about 300 bp in length that act on a promoter to increase its transcription. Examples include the SV40 enhancer on the late side of the replication origin bp 100 to 270, the cytomegalovirus early promoter enhancer, the polyoma enhancer on the late side of the replication origin, and the adenovirus enhancers.

A DNA sequence may be inserted into a vector by a variety of procedures. In general, the DNA sequence is ligated to the desired position in the vector following digestion of the insert and the vector with appropriate restriction endonucleases. Alternatively, blunt ends in both the insert and the vector may be ligated. A variety of cloning techniques are known in the art, e.g., as described in Ausubel and Sambrook. Such procedures and others are deemed to be within the scope of those skilled in the art.

The vector may be in the form of a plasmid, a viral particle, or a phage. Other vectors include chromosomal, non-chromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by, e.g., Sambrook.

Particular bacterial vectors which may be used include the commercially available plasmids comprising genetic elements of the well known cloning vector pBR322 (ATCC 37017), pKK223-3 (Pharmacia Fine Chemicals, Uppsala, Sweden), GEM1 (Promega Biotec, Madison, Wis., USA) pQE70, pQE60, pQE-9 (Qiagen), pD10, psiX174 pBluescript II KS, pNH8A, pNH16a, pNH18A, pNH46A (Stratagene), ptrc99a, pKK223-3, pKK233-3, DR540, pRIT5 (Pharmacia), pKK232-8 and pCM7. Particular eukaryotic vectors include pSV2CAT, pOG44, pXT1, pSG (Stratagene) pSVK3, pBPV, pMSG, and pSVL (Pharmacia). However, any other vector may be used as long as it is replicable and viable in the host cell.

The nucleic acids of the invention can be expressed in expression cassettes, vectors or viruses and transiently or stably expressed in plant cells and seeds. One exemplary transient expression system uses episomal expression systems, e.g., cauliflower mosaic virus (CaMV) viral RNA generated in the nucleus by transcription of an episomal mini-chromosome containing supercoiled DNA, see, e.g., Covey (1990) Proc. Natl. Acad. Sci. USA 87:1633-1637. Alternatively, coding sequences, i.e., all or sub-fragments of sequences of the invention can be inserted into a plant host cell genome becoming an integral part of the host chromosomal DNA. Sense or antisense transcripts can be expressed in this manner. A vector comprising the sequences (e.g., promoters or coding regions) from nucleic acids of the invention can comprise a marker gene that confers a selectable phenotype on a plant cell or a seed. For example, the marker may encode biocide resistance, particularly antibiotic resistance, such as resistance to kanamycin, G418, bleomycin, hygromycin, or herbicide resistance, such as resistance to chlorosulfuron or Basta.

Expression vectors capable of expressing nucleic acids and proteins in plants are well known in the art, and can include, e.g., vectors from Agrobacterium spp., potato virus X (see, e.g., Angell (1997) EMBO J. 16:3675-3684), tobacco mosaic virus (see, e.g., Casper (1996) Gene 173:69-73), tomato bushy stunt virus (see, e.g., Hillman (1989) Virology 169:42-50), tobacco etch virus (see, e.g., Dolja (1997) Virology 234:243-252), bean golden mosaic virus (see, e.g., Morinaga (1993) Microbiol Immunol. 37:471-476), cauliflower mosaic virus (see, e.g., Cecchini (1997) Mol. Plant Microbe Interact. 10:1094-1101), maize Ac/Ds transposable element (see, e.g., Rubin (1997) Mol. Cell. Biol. 17:6294-6302; Kunze (1996) Curr. Top. Microbiol. Immunol. 204:161-194), and the maize suppressor-mutator (Spm) transposable element (see, e.g., Schlappi (1996) Plant Mol. Biol. 32:717-725); and derivatives thereof.

In one aspect, the expression vector can have two replication systems to allow it to be maintained in two organisms, for example in mammalian or insect cells for expression and in a prokaryotic host for cloning and amplification. Furthermore, for integrating expression vectors, the expression vector can contain at least one sequence homologous to the host cell genome. It can contain two homologous sequences which flank the expression construct. The integrating vector can be directed to a specific locus in the host cell by selecting the appropriate homologous sequence for inclusion in the vector. Constructs for integrating vectors are well known in the art.

Expression vectors of the invention may also include a selectable marker gene to allow for the selection of bacterial strains that have been transformed, e.g., genes which render the bacteria resistant to drugs such as ampicillin, chloramphenicol, erythromycin, kanamycin, neomycin and tetracycline. Selectable markers can also include biosynthetic genes, such as those in the histidine, tryptophan and leucine biosynthetic pathways.

In one aspect, the invention provides a xylose isomerase where one amino acid was changed from SEQ ID NO:2, from MTEFFPEI . . . (in SEQ ID NO:2) to MAEFFPEI . . . (SEQ ID NO:6), which is also active in isomerizing glucose and fructose. The first nucleotide residue in the coding sequence for SEQ ID NO:6 (the coding sequence designated SEQ ID NO:5) after the first codon ATG was changed to a “G” to provide a restriction site for cloning, e.g., into an expression cassette, such as a vector, plasmid and the like. In one aspect, SEQ ID NO:5 is used to overexpress the enzyme.

Host Cells and Transformed Cells

The invention also provides a transformed cell comprising a nucleic acid sequence of the invention, e.g., a sequence encoding a xylose isomerase of the invention, or a vector of the invention. The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. The selection of an appropriate host is within the abilities of those skilled in the art. Techniques for transforming a wide variety of higher plant species are well known and described in the technical and scientific literature. See, e.g., Weising (1988) Ann. Rev. Genet. 22:421-477, U.S. Pat. No. 5,750,870.

The vector may be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, I., Basic Methods in Molecular Biology, (1986)).

Where appropriate, the engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

In one aspect, the nucleic acids or vectors of the invention are introduced into the cells for screening, thus, the nucleic acids enter the cells in a manner suitable for subsequent expression of the nucleic acid. The method of introduction is largely dictated by the targeted cell type. Exemplary methods include CaPO₄ precipitation, liposome fusion, lipofection (e.g., LIPOFECTIN™), electroporation, viral infection, etc. The candidate nucleic acids may stably integrate into the genome of the host cell (for example, with retroviral introduction) or may exist either transiently or stably in the cytoplasm (i.e. through the use of traditional plasmids, utilizing standard regulatory sequences, selection markers, etc.). As many pharmaceutically important screens require human or model mammalian cell targets, retroviral vectors capable of transfecting such targets can be used.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment thereof can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

Amplification of Nucleic Acids

In practicing the invention, nucleic acids encoding the polypeptides of the invention, or modified nucleic acids, can be reproduced by, e.g., amplification. The invention provides amplification primer sequence pairs for amplifying nucleic acids encoding xylose isomerases, where the primer pairs are capable of amplifying nucleic acid sequences including the exemplary SEQ ID NO:1, or a subsequence thereof; a sequence as set forth in SEQ ID NO:3, or a subsequence thereof.

In one aspect, the invention provides a nucleic acid amplified by a primer pair of the invention, e.g., a primer pair as set forth by about the first (the 5′) 12, 13, 14, 15, 16, 17, 18, 19, 20, 21, 22, 23, 24, 25, 26, 27, 28, 29 or 30 or more residues of a nucleic acid of the invention, and about the first (the 5′) 15, 16, 17, 18, 19, 20, 21, 22, 23, 24, or 25 residues of the complementary strand; e.g., of the exemplary SEQ ID NO:1; SEQ ID NO:3; SEQ ID NO:5.

The invention provides xylose isomerases generated by amplification, e.g., polymerase chain reaction (PCR), using an amplification primer pair of the invention. The invention provides methods of making xylose isomerases by amplification, e.g., polymerase chain reaction (PCR), using an amplification primer pair of the invention. In one aspect, the amplification primer pair amplifies a nucleic acid from a library, e.g., a gene library, such as an environmental library.

One of skill in the art can design amplification primer sequence pairs for any part of or the full length of these sequences; for example:

The exemplary SEQ ID NO:1 is

| | |
|---|---|
| atgactgagt tctttccaga gatcccgaag atacagtttg aaggtaaaga gagcacaaat | 60 |
| ccatttgcgt tcaagttcta cgatccaaac gaggtgatcg acggaaaacc tctcaaggac | 120 |
| catctgaagt tctcagttgc attctggcac accttcgtga acgaggggag agatcccttc | 180 |
| ggagatccaa cagccgaccg accctggaac aagtacacag accctatgga caaagccttt | 240 |
| gcaagggtgg acgccctctt tgaattctgt gaaaaactca acatcgagta cttctgtttt | 300 |
| cacgacaggg acatagctcc tgaaggaaag actctgaggg agacaaacaa gatcctggac | 360 |
| aaggtcgtgg agaggatcaa agagagaatg aaagacagca acgtaaaact cctctggggt | 420 |
| actgcgaatc tcttttctca tccaaggtac atgcacggtg cggcgacaac ctgtagtgct | 480 |
| gatgtcttcg cctacgcggc agcacaggtg aagaaagccc ttgagatcac aaaagagctt | 540 |
| ggaggagaag ggtacgtctt ttggggtgga agagaagggt acgagacact cctcaacacg | 600 |
| gatctggatc ttgaacttgg aaacctcgct cgcttcctca gaatggctgt ggattacgca | 660 |
| aagaagatag gtttcaacgg ccagtttctc atcgagccta aaccgaagga accaacgaag | 720 |
| catcagtacg acttcgatgt tgcgacggct tacgccttcc tgaagagtca cggtctcgat | 780 |
| gagtatttca aattcaacat cgaagcgaac catgccacac ttgctggtca caccttccag | 840 |
| cacgaactga ggatggcaag aattcttgga aaactcggca gcatcgacgc gaaccagggg | 900 |
| gaccttctgc tcggctggga caccgaccag ttcccaacaa acgtctacga cacaactctt | 960 |
| gccatgtatg aagtgataaa agcgggtggg tttacaaaag gtggtctcaa cttcgatgca | 1020 |
| aaggtgagaa gagcttctta caaggtggaa gatctcttca tcgggcacat agcaggaatg | 1080 |
| gatactttcg cactcggttt caaaatagcc cacaaacttg taaaagacgg tgtgttcgac | 1140 |
| aagttcattg aagaaaaata caaaagtttc agagagggca tcggaaaaga gatcgttgaa | 1200 |
| ggaaaggcag attttgaaaa gctggaagct tatataatag acaaggaaga gatggagctt | 1260 |
| ccatctggaa agcaggagta tttggaaagt ctcctcaaca gctacatagt gaaaacgatc | 1320 |
| tccgagttga ggtga | 1335 |

Thus, an exemplary amplification primer sequence pair is residues 1 to 21 of SEQ ID NO:1 (i.e., atgactgagttctttccagag) and the complementary strand of the last 21 residues of SEQ ID NO:1 (i.e., the complementary strand of acgatctccgagttgaggtga).

The exemplary SEQ ID NO:3 is

| | |
|---|---|
| atgacagaat ttttcccgga aattccaaag atacagttcg aagggaagga aagcaataac | 60 |
| cctcttgcct ttaagttcta cgatccagac gaagtaatcg atggaaaacc tctgaaggac | 120 |
| catttgaaat tctccgttgc tttctggcac acttttgtaa acgaaggtcg agatcccttc | 180 |
| ggtgacccca ctgctgaaag accctggaac aagtattcgg atcccatgga caaagcgttt | 240 |
| gcaagagtgg atgctttatt cgaattctgt gagaaactca atattgaata cttttgtttt | 300 |
| catgacagag acattgcacc cgaagggaaa actctgagag agacgaacaa aattctggac | 360 |
| aaagttgttg agaaaataaa agaacgaatg aaggaaagca atgtgaaact cctttgggga | 420 |
| actgccaatc tgttctcaca tcctcggtac atgcacggtg cggcaactac ttgcagcgcc | 480 |
| gatgtttttg catacgctgc tgcacaggtg aaaaaagcgt tggagattac gaaggaactt | 540 |
| ggaggagaag gatatgtttt ttggggcggt agagaaggat acgaaacctt gctcaacacg | 600 |
| gatttgggat tggaactcga aaacctcgcg aggttcctca gaatggccgt agagtacgca | 660 |
| aagaagatag gttttgatgg acagttcctc atagaaccca aaccaaaaga acccacaaaa | 720 |
| catcagtacg atttcgacgt agcgaccgca tacgccttct tgaaaactca cgatttggat | 780 |
| gaatacttca agttcaacat agaagctaat cacgcaacac tcgctggtca tactttccag | 840 |
| catgaattga gaatggccag aatcctcgga aaattcggaa gtatcgacgc aaatcaaggc | 900 |
| gatcttctgt tgggatggga caccgatcaa tttccaacga acgtatacga tacaactctt | 960 |
| gccatgtacg aggttataaa agcagggggt ttcacaaaag gtggtctcaa cttcgacgcc | 1020 |
| aaagtgagac gtgcttctta caaggtagag gatctcttca tcgggcatat agtaggaata | 1080 |
| gacactttcg cactcggttt caagatagcc tacaaacttg taaaagacgg cgtattcgac | 1140 |
| agattcgttg aggaaaaata cagaagtttc agagaaggta ttggaaaaga aatattggaa | 1200 |
| ggaaaagcag attttgaaaa actagaatcg tatataatag acaaagaaga tgttgaactt | 1260 |
| ccatctggaa aacaggagta tcttgaaagt ttgctcaaca gctatatcgt gaagaccgta | 1320 |
| tcagaactga ggtga | 1335 |

Thus, an exemplary amplification primer sequence pair is residues 1 to 21 of SEQ ID NO:3 (i.e., atgacagaatttttcccggaa) and the complementary strand of the last 21 residues of SEQ ID NO:3 (i.e., the complementary strand of accgtatcagaactgaggtga).

The exemplary SEQ ID NO:5 is

| | |
|---|---|
| atggctgagt tctttccaga gatcccgaag atacagtttg aaggtaaaga gagcacaaat | 60 |
| ccatttgcgt tcaagttcta cgatccaaac gaggtgatcg acggaaaacc tctcaaggac | 120 |
| catctgaagt tctcagttgc attctggcac accttcgtga acgaggggag agatcccttc | 180 |
| ggagatccaa cagccgaccg accctggaac aagtacacag accctatgga caaagccttt | 240 |
| gcaagggtgg acgccctctt tgaattctgt gaaaaactca acatcgagta cttctgtttt | 300 |
| cacgacaggg acatagctcc tgaaggaaag actctgaggg agacaaacaa gatcctggac | 360 |
| aaggtcgtgg agaggatcaa agagagaatg aaagacagca acgtaaaact cctctggggt | 420 |
| actgcgaatc tcttttctca tccaaggtac atgcacggtg cggcgacaac ctgtagtgct | 480 |
| gatgtcttcg cctacgcggc agcacaggtg aagaaagccc ttgagatcac aaaagagctt | 540 |
| ggaggagaag ggtacgtctt ttggggtgga agagaagggt acgagacact cctcaacacg | 600 |
| gatctggatc ttgaacttgg aaacctcgct cgcttcctca gaatggctgt ggattacgca | 660 |
| aagaagatag gtttcaacgg ccagtttctc atcgagccta aaccgaagga accaacgaag | 720 |
| catcagtacg acttcgatgt tgcgacggct tacgccttcc tgaagagtca cggtctcgat | 780 |
| gagtatttca aattcaacat cgaagcgaac catgccacac ftgctggtca caccttccag | 840 |
| cacgaactga ggatggcaag aattcttgga aaactcggca gcatcgacgc gaaccagggg | 900 |
| gaccttctgc tcggctggga caccgaccag ttcccaacaa acgtctacga cacaactctt | 960 |
| gccatgtatg aagtgataaa agcgggtggg tttacaaaag gtggtctcaa cttcgatgca | 1020 |
| aaggtgagaa gagcttctta caaggtggaa gatctcttca tcgggcacat agcaggaatg | 1080 |
| gatactttcg cactcggttt caaaatagcc cacaaacttg taaaagacgg tgtgttcgac | 1140 |
| aagttcattg aagaaaaata caaaagtttc agagagggca tcggaaaaga gatcgttgaa | 1200 |
| ggaaaggcag attttgaaaa gctggaagct tatataatag acaaggaaga gatggagctt | 1260 |
| ccatctggaa agcaggagta tttggaaagt ctcctcaaca gctacatagt gaaaacgatc | 1320 |
| tccgagttga ggtga | 1335 |

Amplification reactions can also be used to quantify the amount of nucleic acid in a sample (such as the amount of message in a cell sample), label the nucleic acid (e.g., to apply it to an array or a blot), detect the nucleic acid, or quantify the amount of a specific nucleic acid in a sample. In one aspect of the invention, message isolated from a cell or a cDNA library are amplified.

The skilled artisan can select and design suitable oligonucleotide amplification primers. Amplification methods are also well known in the art, and include, e.g., polymerase chain reaction, PCR (see, e.g., PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564.

Determining the Degree of Sequence Identity

The invention provides nucleic acids comprising sequences having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to an exemplary nucleic acid of the invention, e.g., SEQ ID NO:1; SEQ ID NO:3; SEQ ID NO:5. In one aspect, the invention provides nucleic acids having at least 96%/o sequence identity to SEQ ID NO:1 or SEQ ID NO:5, or nucleic acids having at least 95% sequence identity to SEQ ID NO:3. In alternative embodiments, the invention provides nucleic acids and polypeptides having at least 99%, 98%, 97% or 96% sequence identity (homology) to SEQ ID NO:1, SEQ ID NO:2, SEQ ID NO:3, SEQ ID NO:4, SEQ ID NO:5, SEQ ID NO:6. In alternative aspects, the sequence identify can be over a region of at least about 5, 10, 20, 30, 40, 50, 100, 150, 200, 250, 300, 350, 400, 450, 500, 550, 600, 650, 700, 750, 800, 850, 900, 950, 1000, or more consecutive residues, or the full length of the nucleic acid or polypeptide. The extent of sequence identity (homology) may be determined using any computer program and associated parameters, including those described herein, such as BLAST 2.2.2. or FASTA version 3.0t78, with the default parameters.

Homologous sequences also include RNA sequences in which uridines replace the thymines in the nucleic acid sequences. The homologous sequences may be obtained using any of the procedures described herein or may result from the correction of a sequencing error. It will be appreciated that the nucleic acid sequences as set forth herein can be represented in the traditional single character format (see, e.g., Stryer, Lubert. Biochemistry, 3rd Ed., W. H Freeman & Co., New York) or in any other format which records the identity of the nucleotides in a sequence.

Various sequence comparison programs identified herein are used in this aspect of the invention. Protein and/or nucleic acid sequence identities (homologies) may be evaluated using any of the variety of sequence comparison algorithms and programs known in the art. Such algorithms and programs include, but are not limited to, TBLASTN, BLASTP, FASTA, TFASTA, and CLUSTALW (Pearson and Lipman, Proc. Natl. Acad. Sci. USA 85(8):2444-2448, 1988; Altschul et al., J. Mol. Biol. 215(3):403-410, 1990; Thompson et al., Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993).

Homology or identity can be measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various deletions, substitutions and other modifications. The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence (an exemplary sequence SEQ ID NO:1, SEQ ID NO:2, SEQ ID NO:3, SEQ ID NO:4, SEQ ID NO:5, SEQ ID NO:6 to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the numbers of contiguous residues. For example, in alternative aspects of the invention, continugous residues ranging anywhere from 20 to the full length of an exemplary polypeptide or nucleic acid sequence of the invention, e.g., SEQ ID NO:1, SEQ ID NO:2, SEQ ID NO:3, SEQ ID NO:4, SEQ ID NO:5, SEQ ID NO:6, are compared to a reference sequence of the same number of contiguous positions after the two sequences are optimally aligned. If the reference sequence has the requisite sequence identity to an exemplary polypeptide or nucleic acid sequence of the invention, e.g., 95%, 96%, 97%, 98%, 99% sequence identity to SEQ ID NO:1, SEQ ID NO:2, SEQ ID NO:3, SEQ ID NO:4, SEQ ID NO:5, SEQ ID NO:6, that sequence is within the scope of the invention. In alternative embodiments, subsequences ranging from about 20 to 600, about 50 to 200, and about 100 to 150 are compared to a reference sequence of the same number of contiguous positions after the two sequences are optimally aligned. Methods of alignment of sequence for comparison are well known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of person & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. Other algorithms for determining homology or identity include, for example, in addition to a BLAST program (Basic Local Alignment Search Tool at the National Center for Biological Information), ALIGN, AMAS (Analysis of Multiply Aligned Sequences), AMPS (Protein Multiple Sequence Alignment), ASSET (Aligned Segment Statistical Evaluation Tool), BANDS, BESTSCOR, BIOSCAN (Biological Sequence Comparative Analysis Node), BLIMPS (BLocks IMProved Searcher), FASTA, Intervals & Points, BMB, CLUSTAL V, CLUSTAL W, CONSENSUS, LCONSENSUS, WCONSENSUS, Smith-Waterman algorithm, DARWIN, Las Vegas algorithm, FNAT (Forced Nucleotide Alignment Tool), Framealign, Framesearch, DYNAMIC, FILTER, FSAP (Fristensky Sequence Analysis Package), GAP (Global Alignment Program), GENAL, GIBBS, GenQuest, ISSC (Sensitive Sequence Comparison), LALIGN (Local Sequence Alignment), LCP (Local Content Program), MACAW (Multiple Alignment Construction & Analysis Workbench), MAP (Multiple Alignment Program), MBLKP, MBLKN, PIMA (Pattern-Induced Multi-sequence Alignment), SAGA (Sequence Alignment by Genetic Algorithm) and WHAT-IF. Such alignment programs can also be used to screen genome databases to identify polynucleotide sequences having substantially identical sequences. A number of genome databases are available, for example, a substantial portion of the human genome is available as part of the Human Genome Sequencing Project (Gibbs, 1995). Several genomes have been sequenced, e.g., M. genitalium (Fraser et al., 1995), M. jannaschii (Bult et al., 1996), H. influenzae (Fleischmann et al., 1995), E. coli (Blattner et al., 1997), and yeast (S. cerevisiae) (Mewes et al., 1997), and D. melanogaster (Adams et al., 2000). Significant progress has also been made in sequencing the genomes of model organism, such as mouse, C. elegans, and Arabadopsis sp. Databases containing genomic information annotated with some functional information are maintained by different organization, and are accessible via the internet.

BLAST, BLAST 2.0 and BLAST 2.2.2 algorithms are also used to practice the invention. They are described, e.g., in Altschul (1977) Nuc. Acids Res. 25:3389-3402; Altschul (1990) J. Mol. Biol. 215:403-410. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul (1990) supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectations (E) of 10, and the BLOSUM62 scoring matrix (see Henikoff & Henikoff (1989) Proc. Natl. Acad. Sci. USA 89:10915) alignments (B) of 50, expectation (E) of 10, M=5, N=−4, and a comparison of both strands. The BLAST algorithm also performs a statistical analysis of the similarity between two sequences (see, e.g., Karlin & Altschul (1993) Proc. Natl. Acad. Sci. USA 90:5873). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, or less than about 0.01, or less than about 0.001. In one aspect, protein and nucleic acid sequence homologies are evaluated using the Basic Local Alignment Search Tool (“BLAST”). For example, five specific BLAST programs can be used to perform the following task: (1) BLASTP and BLAST3 compare an amino acid query sequence against a protein sequence database; (2) BLASTN compares a nucleotide query sequence against a nucleotide sequence database; (3) BLASTX compares the six-frame conceptual translation products of a query nucleotide sequence (both strands) against a protein sequence database; (4) TBLASTN compares a query protein sequence against a nucleotide sequence database translated in all six reading frames (both strands); and, (5) TBLASTX compares the six-frame translations of a nucleotide query sequence against the six-frame translations of a nucleotide sequence database. The BLAST programs identify homologous sequences by identifying similar segments, which are referred to herein as “high-scoring segment pairs,” between a query amino or nucleic acid sequence and a test sequence which can be obtained from a protein or nucleic acid sequence database. High-scoring segment pairs are preferably identified (i.e., aligned) by means of a scoring matrix, many of which are known in the art. The scoring matrix can used is the BLOSUM62 matrix (Gonnet (1992) Science 256:1443-1445; Henikoff and Henikoff, Proteins 17:49-61, 1993). The PAM or PAM250 matrices may also be used (see, e.g., Schwartz and Dayhoff, eds., 1978, Matrices for Detecting Distance Relationships: Atlas of Protein Sequence and Structure, Washington: National Biomedical Research Foundation).

In one aspect of the invention, to determine if a nucleic acid has the requisite sequence identity to be within the scope of the invention, the NCBI BLAST 2.2.2 programs is used, default options to blastp. There are about 38 setting options in the BLAST 2.2.2 program. In this exemplary aspect of the invention, all default values are used except for the default filtering setting (i.e., all parameters set to default except filtering which is set to OFF); in its place a “-F F” setting is used, which disables filtering. Use of default filtering often results in Karlin-Altschul violations due to short length of sequence.

The default values used in this exemplary aspect of the invention include:

“Filter for low complexity: ON

Word Size: 3

Matrix: Blosum62

Gap Costs: Existence: 11

Extension:1″

Other default settings are: filter for low complexity OFF, word size of 3 for protein, BLOSUM62 matrix, gap existence penalty of −11 and a gap extension penalty of −1. An exemplary NCBI BLAST 2.2.2 program setting is set forth in Example 1, below. Note that the “−W” option defaults to 0. This means that, if not set, the word size defaults to 3 for proteins and 11 for nucleotides.

Computer Systems and Computer Program Products

To determine and identify sequence identities, structural homologies, motifs and the like in silico, the sequence of the invention can be stored, recorded, and manipulated on any medium which can be read and accessed by a computer. Accordingly, the invention provides computers, computer systems, computer readable mediums, computer programs products and the like recorded or stored thereon the nucleic acid and polypeptide sequences of the invention. As used herein, the words “recorded” and “stored” refer to a process for storing information on a computer medium. A skilled artisan can readily adopt any known methods for recording information on a computer readable medium to generate manufactures comprising one or more of the nucleic acid and/or polypeptide sequences of the invention.

Another aspect of the invention is a computer readable medium having recorded thereon at least one nucleic acid and/or polypeptide sequence of the invention. Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of other media known to those skilled in the art.

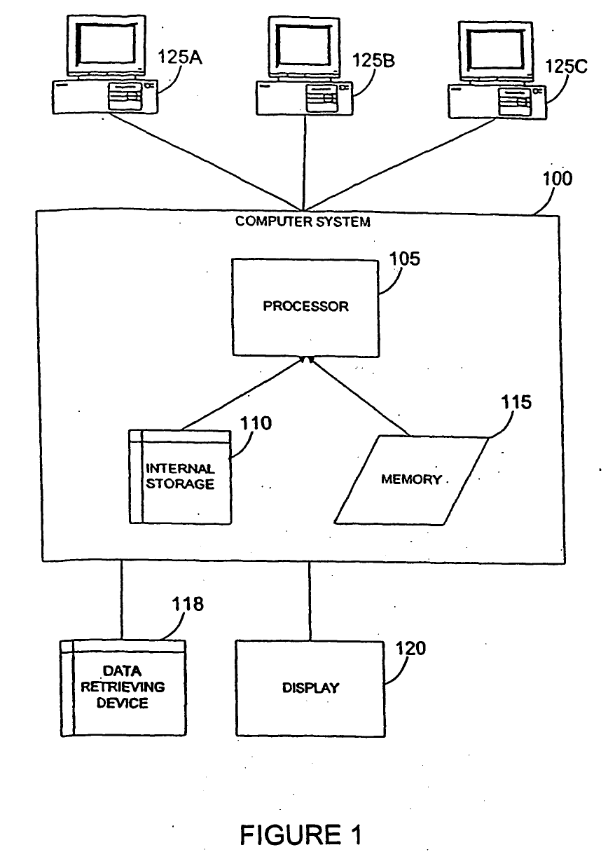

Aspects of the invention include systems (e.g., internet based systems), particularly computer systems, which store and manipulate the sequences and sequence information described herein. One example of a computer system 100 is illustrated in block diagram form in FIG. 1. As used herein, “a computer system” refers to the hardware components, software components, and data storage components used to analyze a nucleotide or polypeptide sequence of the invention. The computer system 100 can include a processor for processing, accessing and manipulating the sequence data. The processor 105 can be any well-known type of central processing unit, such as, for example, the Pentium III from Intel Corporation, or similar processor from Sun, Motorola, Compaq, AMD or International Business Machines. The computer system 100 is a general purpose system that comprises the processor 105 and one or more internal data storage components 110 for storing data, and one or more data retrieving devices for retrieving the data stored on the data storage components. A skilled artisan can readily appreciate that any one of the currently available computer systems are suitable.

In one aspect, the computer system 100 includes a processor 105 connected to a bus which is connected to a main memory 115 (which can be implemented as RAM) and one or more internal data storage devices 110, such as a hard drive and/or other computer readable media having data recorded thereon. The computer system 100 can further include one or more data retrieving device 118 for reading the data stored on the internal data storage devices 110. The data retrieving device 118 may represent, for example, a floppy disk drive, a compact disk drive, a magnetic tape drive, or a modem capable of connection to a remote data storage system (e.g., via the internet) etc. In some embodiments, the internal data storage device 110 is a removable computer readable medium such as a floppy disk, a compact disk, a magnetic tape, etc. containing control logic and/or data recorded thereon. The computer system 100 may advantageously include or be programmed by appropriate software for reading the control logic and/or the data from the data storage component once inserted in the data retrieving device. The computer system 100 includes a display 120 which is used to display output to a computer user. It should also be noted that the computer system 100 can be linked to other computer systems 125a-c in a network or wide area network to provide centralized access to the computer system 100. Software for accessing and processing the nucleotide or amino acid sequences of the invention can reside in main memory 115 during execution. In some aspects, the computer system 100 may further comprise a sequence comparison algorithm for comparing a nucleic acid sequence of the invention. The algorithm and sequence(s) can be stored on a computer readable medium. A “sequence comparison algorithm” refers to one or more programs which are implemented (locally or remotely) on the computer system 100 to compare a nucleotide sequence with other nucleotide sequences and/or compounds stored within a data storage means. For example, the sequence comparison algorithm may compare the nucleotide sequences of the invention stored on a computer readable medium to reference sequences stored on a computer readable medium to identify homologies or structural motifs.

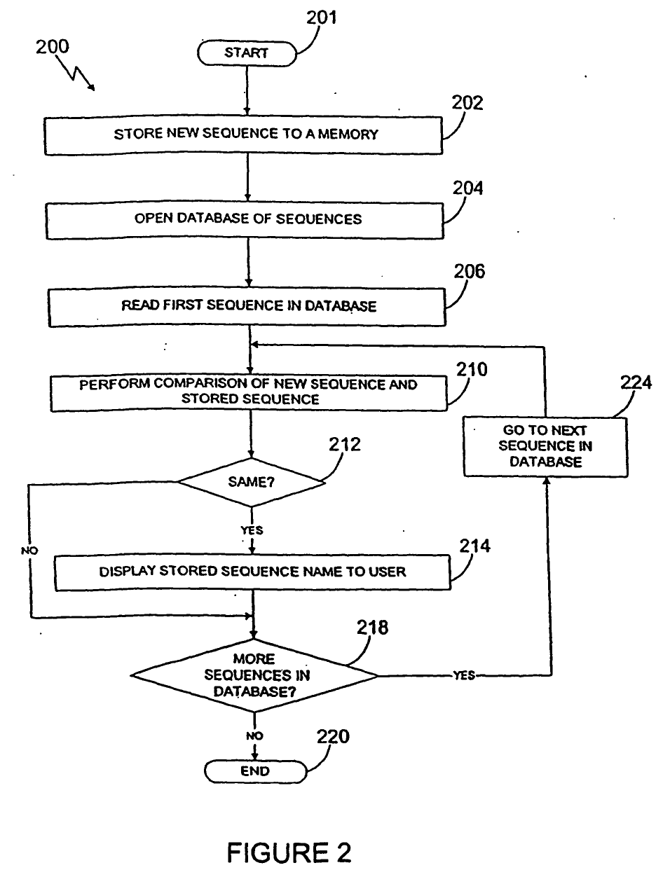

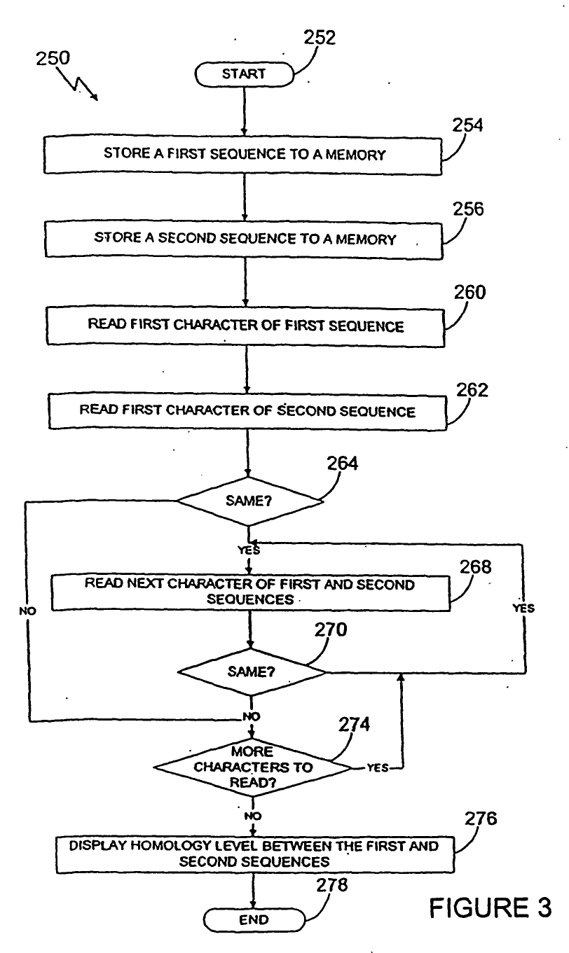

The parameters used with the above algorithms may be adapted depending on the sequence length and degree of homology studied. In some aspects, the parameters may be the default parameters used by the algorithms in the absence of instructions from the user. FIG. 2 is a flow diagram illustrating one aspect of a process 200 for comparing a new nucleotide or protein sequence with a database of sequences in order to determine the homology levels between the new sequence and the sequences in the database. The database of sequences can be a private database stored within the computer system 100, or a public database such as GENBANK that is available through the Internet. The process 200 begins at a start state 201 and then moves to a state 202 wherein the new sequence to be compared is stored to a memory in a computer system 100. As discussed above, the memory could be any type of memory, including RAM or an internal storage device. The process 200 then moves to a state 204 wherein a database of sequences is opened for analysis and comparison. The process 200 then moves to a state 206 wherein the first sequence stored in the database is read into a memory on the computer. A comparison is then performed at a state 210 to determine if the first sequence is the same as the second sequence. It is important to note that this step is not limited to performing an exact comparison between the new sequence and the first sequence in the database. Well-known methods are known to those of skill in the art for comparing two nucleotide or protein sequences, even if they are not identical. For example, gaps can be introduced into one sequence in order to raise the homology level between the two tested sequences. The parameters that control whether gaps or other features are introduced into a sequence during comparison are normally entered by the user of the computer system. Once a comparison of the two sequences has been performed at the state 210, a determination is made at a decision state 210 whether the two sequences are the same. Of course, the term “same” is not limited to sequences that are absolutely identical. Sequences that are within the homology parameters entered by the user will be marked as “same” in the process 200. If a determination is made that the two sequences axe the same, the process 200 moves to a state 214 wherein the name of the sequence from the database is displayed to the user. This state notifies the user that the sequence with the displayed name fulfills the homology constraints that were entered. Once the name of the stored sequence is displayed to the user, the process 200 moves to a decision state 218 wherein a determination is made whether more sequences exist in the database. If no more sequences exist in the database, then the process 200 terminates at an end state 220. However, if more sequences do exist in the database, then the process 200 moves to a state 224 wherein a pointer is moved to the next sequence in the database so that it can be compared to the new sequence. In this manner, the new sequence is aligned and compared with every sequence in the database. It should be noted that if a determination had been made at the decision state 212 that the sequences were not homologous, then the process 200 would move immediately to the decision state 218 in order to determine if any other sequences were available in the database for comparison. Accordingly, one aspect of the invention is a computer system comprising a processor, a data storage device having stored thereon a nucleic acid sequence of the invention and a sequence comparer for conducting the comparison. The sequence comparer may indicate a homology level between the sequences compared or identify structural motifs, or it may identify structural motifs in sequences which are Compared to these nucleic acid codes and polypeptide codes. FIG. 3 is a flow diagram illustrating one embodiment of a process 250 in a computer for determining whether two sequences are homologous. The process 250 begins at a start state 252 and then moves to a state 254 wherein a first sequence to be compared is stored to a memory. The second sequence to be compared is then stored to a memory at a state 256. The process 250 then moves to a state 260 wherein the first character in the first sequence is read and then to a state 262 wherein the first character of the second sequence is read. It should be understood that if the sequence is a nucleotide sequence, then the character would normally be either A, T, C, G or U. If the sequence is a protein sequence, then it can be a single letter amino acid code so that the first and sequence sequences can be easily compared. A determination is then made at a decision state 264 whether the two characters are the same. If they are the same, then the process 250 moves to a state 268 wherein the next characters in the first and second sequences are read. A determination is then made whether the next characters are the same. If they are, then the process 250 continues this loop until two characters are not the same. If a determination is made that the next two characters are not the same, the process 250 moves to a decision state 274 to determine whether there are any more characters either sequence to read. If there are not any more characters to read, then the process 250 moves to a state 276 wherein the level of homology between the first and second sequences is displayed to the user. The level of homology is determined by calculating the proportion of characters between the sequences that were the same out of the total number of sequences in the first sequence. Thus, if every character in a first 100 nucleotide sequence aligned with an every character in a second sequence, the homology level would be 100%.

Alternatively, the computer program can compare a reference sequence to a sequence of the invention to determine whether the sequences differ at one or more positions. The program can record the length and identity of inserted, deleted or substituted nucleotides or amino acid residues with respect to the sequence of either the reference or the invention. The computer program may be a program which determines whether a reference sequence contains a single nucleotide polymorphism (SNP) with respect to a sequence of the invention, or, whether a sequence of the invention comprises a SNP of a known sequence. Thus, in some aspects, the computer program is a program which identifies SNPs. The method may be implemented by the computer systems described above and the method illustrated in FIG. 3. The method can be performed by reading a sequence of the invention and the reference sequences through the use of the computer program and identifying differences with the computer program.

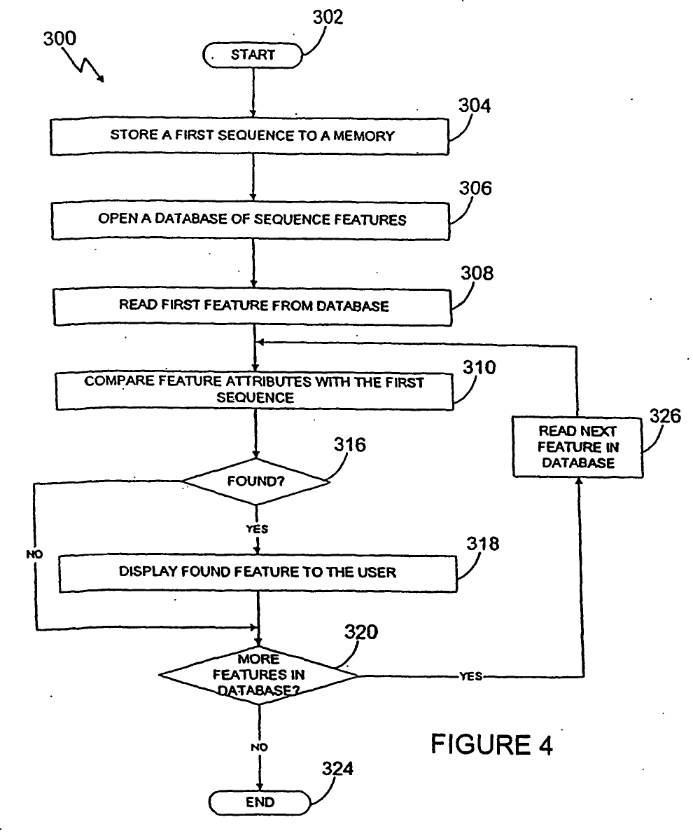

In other aspects the computer based system comprises an identifier for identifying features within a nucleic acid or polypeptide of the invention. An “identifier” refers to one or more programs which identifies certain features within a nucleic acid sequence. For example, an identifier may comprise a program which identifies an open reading frame (ORF) in a nucleic acid sequence. FIG. 4 is a flow diagram illustrating one aspect of an identifier process 300 for detecting the presence of a feature in a sequence. The process 300 begins at a start state 302 and then moves to a state 304 wherein a first sequence that is to be checked for features is stored to a memory 115 in the computer system 100. The process 300 then moves to a state 306 wherein a database of sequence features is opened. Such a database would include a list of each feature's attributes along with the name of the feature. For example, a feature name could be “Initiation Codon” and the attribute would be “ATG”. Another example would be the feature name “TAATAA Box” and the feature attribute would be “TAATAA”. An example of such a database is produced by the University of Wisconsin Genetics Computer Group. Alternatively, the features may be structural polypeptide motifs such as alpha helices, beta sheets, or functional polypeptide motifs such as enzymatic active sites, helix-turn-helix motifs or other motifs known to those skilled in the art. Once the database of features is opened at the state 306, the process 300 moves to a state 308 wherein the first feature is read from the database. A comparison of the attribute of the first feature with the first sequence is then made at a state 310. A determination is then made at a decision state 316 whether the attribute of the feature was found in the first sequence. If the attribute was found, then the process 300 moves to a state 318 wherein the name of the found feature is displayed to the user. The process 300 then moves to a decision state 320 wherein a determination is made whether move features exist in the database. If no more features do exist, then the process 300 terminates at an end state 324. However, if more features do exist in the database, then the process 300 reads the next sequence feature at a state 326 and loops back to the state 310 wherein the attribute of the next feature is compared against the first sequence. If the feature attribute is not found in the first sequence at the decision state 316, the process 300 moves directly to the decision state 320 in order to determine if any more features exist in the database. Thus, in one aspect, the invention provides a computer program that identifies open reading frames (ORFs).

A polypeptide or nucleic acid sequence of the invention may be stored and manipulated in a variety of data processor programs in a variety of formats. For example, a sequence can be stored as text in a word processing file, such as MicrosoftWORD or WORDPERFECT or as an ASCII file in a variety of database programs familiar to those of skill in the art, such as DB2, SYBASE, or ORACLE. In addition, many computer programs and databases may be used as sequence comparison algorithms, identifiers, or sources of reference nucleotide sequences or polypeptide sequences to be compared to a nucleic acid sequence of the invention. The programs and databases used to practice the invention include, but are not limited to: MacPattern (EMBL), DiscoveryBase (Molecular Applications Group), GeneMine (Molecular Applications Group), Look (Molecular Applications Group), MacLook (Molecular Applications Group), BLAST and BLAST2 (NCBI), BLASTN and BLASTX (Altschul et al, J. Mol. Biol. 215: 403, 1990), FASTA (Pearson and Lipman, Proc. Natl. Acad. Sci. USA, 85: 2444, 1988), FASTDB (Brutlag et al. Comp. App. Biosci. 6:237-245, 1990), Catalyst (Molecular Simulations Inc.), Catalyst/SHAPE (Molecular Simulations Inc.), Cerius2.DBAccess (Molecular Simulations Inc.), HypoGen (Molecular Simulations Inc.), Insight II, (Molecular Simulations Inc.), Discover (Molecular Simulations Inc.), CHARMm (Molecular Simulations Inc.), Felix (Molecular Simulations Inc.), DelPhi, (Molecular Simulations Inc.), QuanteMM, (Molecular Simulations Inc.), Homology (Molecular Simulations Inc.), Modeler (Molecular Simulations Inc.), ISIS (Molecular Simulations Inc.), Quanta/Protein Design (Molecular Simulations Inc.), WebLab (Molecular Simulations Inc.), WebLab Diversity Explorer (Molecular Simulations Inc.), Gene Explorer (Molecular Simulations Inc.), SeqFold (Molecular Simulations Inc.), the MDL Available Chemicals Directory database, the MDL Drug Data Report data base, the Comprehensive Medicinal Chemistry database, Derwent's World Drug Index database, the BioByteMasterFile database, the Genbank database, and the Genseqn database. Many other programs and data bases would be apparent to one of skill in the art given the present disclosure.

Motifs which may be detected using the above programs include sequences encoding leucine zippers, helix-turn-helix motifs, glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites.

Hybridization of Nucleic Acids

The invention provides isolated or recombinant nucleic acids that hybridize under stringent conditions to an exemplary sequence of the invention, e.g., a sequence as set forth in SEQ ID NO:1, SEQ ID NO:3, SEQ ID NO:5, or a nucleic acid that encodes a polypeptide of the invention or fragments or subsequences thereof. The stringent conditions can be highly stringent conditions, medium stringent conditions, low stringent conditions, including the high and reduced stringency conditions described herein.

In alternative embodiments, nucleic acids of the invention as defined by their ability to hybridize under stringent conditions can be between about five residues and the full length of nucleic acid of the invention; e.g., they can be at least 5, 10, 15, 20, 25, 30, 35, 40, 50, 55, 60, 65, 70, 75, 80, 90, 100, 150, 200, 250, 300, 350, 400, 450, 500, 550, 600, 650, 700, 750, 800, 850, 900, 950, 1000, or more, residues in length. Nucleic acids shorter than full length are also included. These nucleic acids can be useful as, e.g., hybridization probes, labeling probes, PCR oligonucleotide probes, iRNA, antisense or sequences encoding antibody binding peptides (epitopes), motifs, active sites and the like.

In one aspect, nucleic acids of the invention are defined by their ability to hybridize under high stringency comprises conditions of about 50% formamide at about 37° C. to 42° C. In one aspect, nucleic acids of the invention are defined by their ability to hybridize under reduced stringency comprising conditions in about 35% to 25% formamide at about 30° C. to 35° C.

Alternatively, nucleic acids of the invention are defined by their ability to hybridize under high stringency comprising conditions at 42° C. in 50% formamide, 5×SSPE, 0.3% SDS, and a repetitive sequence blocking nucleic acid, such as cot-1 or salmon sperm DNA (e.g., 200 n/ml sheared and denatured salmon sperm DNA). In one aspect, nucleic acids of the invention are defined by their ability to hybridize under reduced stringency conditions comprising 35% formamide at a reduced temperature of 35° C.

Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide.

The temperature range corresponding to a particular level of stringency can be further narrowed by calculating the purine to pyrimidine ratio of the nucleic acid of interest and adjusting the temperature accordingly. Nucleic acids of the invention are also defined by their ability to hybridize under high, medium, and low stringency conditions as set forth in Ausubel and Sambrook. Variations on the above ranges and conditions are well known in the art. Hybridization conditions are discussed further, below.

The above procedure may be modified to identify nucleic acids having decreasing levels of homology to the probe sequence. For example, to obtain nucleic acids of decreasing homology to the detectable probe, less stringent conditions may be used. For example, the hybridization temperature may be decreased in increments of 5° C. from 68° C. to 42° C. in a hybridization buffer having a Na⁺ concentration of approximately 1M. Following hybridization, the filter may be washed with 2×SSC, 0.5% SDS at the temperature of hybridization. These conditions are considered to be “moderate” conditions above 50° C. and “low” conditions below 50° C. A specific example of “moderate” hybridization conditions is when the above hybridization is conducted at 55° C. A specific example of “low stringency” hybridization conditions is when the above hybridization is conducted at 45° C.

Alternatively, the hybridization may be carried out in buffers, such as 6×SSC, containing formamide at a temperature of 42° C. In this case, the concentration of formamide in the hybridization buffer may be reduced in 5% increments from 50% to 0% to identify clones having decreasing levels of homology to the probe. Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide.

However, the selection of a hybridization format is not critical—it is the stringency of the wash conditions that set forth the conditions which determine whether a nucleic acid is within the scope of the invention. Wash conditions used to identify nucleic acids within the scope of the invention include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. See Sambrook, Tijssen and Ausubel for a description of SSC buffer and equivalent conditions.

These methods may be used to isolate nucleic acids of the invention.

Oligonucleotides Probes and Methods for Using Them

The invention also provides nucleic acid probes for identifying nucleic acids encoding a polypeptide with a xylose isomerase activity. In one aspect, the probe comprises at least 10 consecutive bases of a nucleic acid of the invention. Alternatively, a probe of the invention can be at least about 5, 6, 7, 8, 9, 10, 15, 20, 25, 30, 35, 40, 45, 50, 60, 70, 80, 90, 100, 110, 120, 130, 150 or about 10 to 50, about 20 to 60 about 30 to 70, consecutive bases of a sequence as set forth in a nucleic acid of the invention. The probes identify a nucleic acid by binding and/or hybridization. The probes can be used in arrays of the invention, see discussion below, including, e.g., capillary arrays. The probes of the invention can also be used to isolate other nucleic acids or polypeptides.

The probes of the invention can be used to determine whether a biological sample, such as a soil sample, contains an organism having a nucleic acid sequence of the invention or an organism from which the nucleic acid was obtained. In such procedures, a biological sample potentially harboring the organism from which the nucleic acid was isolated is obtained and nucleic acids are obtained from the sample. The nucleic acids are contacted with the probe under conditions which permit the probe to specifically hybridize to any complementary sequences present in the sample. Where necessary, conditions which permit the probe to specifically hybridize to complementary sequences may be determined by placing the probe in contact with complementary sequences from samples known to contain the complementary sequence, as well as control sequences which do not contain the complementary sequence. Hybridization conditions, such as the salt concentration of the hybridization buffer, the formamide concentration of the hybridization buffer, or the hybridization temperature, may be varied to identify conditions which allow the probe to hybridize specifically to complementary nucleic acids (see discussion on specific hybridization conditions).

If the sample contains the organism from which the nucleic acid was isolated, specific hybridization of the probe is then detected. Hybridization may be detected by labeling the probe with a detectable agent such as a radioactive isotope, a fluorescent dye or an enzyme capable of catalyzing the formation of a detectable product Many methods for using the labeled probes to detect the presence of complementary nucleic acids in a sample are familiar to those skilled in the art. These include Southern Blots, Northern Blots, colony hybridization procedures, and dot blots. Protocols for each of these procedures are provided in Ausubel and Sambrook.

Alternatively, more than one probe (at least one of which is capable of specifically hybridizing to any complementary sequences which are present in the nucleic acid sample), may be used in an amplification reaction to determine whether the sample contains an organism containing a nucleic acid sequence of the invention (e.g., an organism from which the nucleic acid was isolated). In one aspect, the probes comprise oligonucleotides. In one aspect, the amplification reaction may comprise a PCR reaction. PCR protocols are described in Ausubel and Sambrook (see discussion on amplification reactions). In such procedures, the nucleic acids in the sample are contacted with the probes, the amplification reaction is performed, and any resulting amplification product is detected. The amplification product may be detected by performing gel electrophoresis on the reaction products and staining the gel with an intercalator such as ethidium bromide. Alternatively, one or more of the probes may be labeled with a radioactive isotope and the presence of a radioactive amplification product may be detected by autoradiography after gel electrophoresis.

Probes derived from sequences near the 3′ or 5′ ends of a nucleic acid sequence of the invention can also be used in chromosome walking procedures to identify clones containing additional, e.g., genomic sequences. Such methods allow the isolation of genes which encode additional proteins of interest from the host organism.

In one aspect, nucleic acid sequences of the invention are used as probes to identify and isolate related nucleic acids.

In some aspects, the so-identified related nucleic acids may be cDNAs or genomic DNAs from organisms other than the one from which the nucleic acid of the invention was first isolated. In such procedures, a nucleic acid sample is contacted with the probe under conditions which permit the probe to specifically hybridize to related sequences. Hybridization of the probe to nucleic acids from the related organism is then detected using any of the methods described above.

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter. Hybridization may be carried out under conditions of low stringency, moderate stringency or high stringency. As an example of nucleic acid hybridization, a polymer membrane containing immobilized denatured nucleic acids is first prehybridized for 30 minutes at 45° C. in a solution consisting of 0.9 M NaCl, 50 mM NaH₂PO4, pH 7.0, 5.0 mM Na₂EDTA, 0.5% SDS, 10× Denhardt's, and 0.5 mg/ml polyriboadenylic acid. Approximately 2×107 cpm (specific activity 4-9×108 cpm/ug) of 32P end-labeled oligonucleotide probe are then added to the solution. After 12-16 hours of incubation, the membrane is washed for 30 minutes at room temperature (RT) in 1×SET (150 mM NaCl, 20 mM Tris hydrochloride, pH 7.8, 1 mM Na2EDTA) containing 0.5% SDS, followed by a 30 minute wash in fresh 1×SET at Tm-10° C. for the oligonucleotide probe. The membrane is then exposed to auto-radiographic film for detection of hybridization signals.

By varying the stringency of the hybridization conditions used to identify nucleic acids, such as cDNAs or genomic DNAs, which hybridize to the detectable probe, nucleic acids having different levels of homology to the probe can be identified and isolated. Stringency may be varied by conducting the hybridization at varying temperatures below the melting temperatures of the probes. The melting temperature, Tm, is the temperature (under defined ionic strength and pH) at which 50% of the target sequence hybridizes to a perfectly complementary probe. Very stringent conditions are selected to be equal to or about 5° C. lower than the Tm for a particular probe. The melting temperature of the probe may be calculated using the following exemplary formulas. For probes between 14 and 70 nucleotides in length the melting temperature (Tm) is calculated using the formula: Tm=81.5+16.6(log [Na+])+0.41(fraction G+C)−(600/N) where N is the length of the probe. If the hybridization is carried out in a solution containing formamide, the melting temperature may be calculated using the equation: Tm=81.5+16.6(log [Na+])+0.41(fraction G+C)−(0.63% formamide)−(600/N) where N is the length of the probe. Prehybridization may be carried out in 6×SSC, 5× Denhardt's reagent, 0.5% SDS, 100 μg denatured fragmented salmon sperm DNA or 6×SSC, 5× Denhardt's reagent, 0.5% SDS, 100 μg denatured fragmented salmon sperm DNA, 50% formamide. Formulas for SSC and Denhardt's and other solutions are listed, e.g., in Sambrook.

Hybridization is conducted by adding the detectable probe to the prehybridization solutions listed above. Where the probe comprises double stranded DNA, it is denatured before addition to the hybridization solution. The filter is contacted with the hybridization solution for a sufficient period of time to allow the probe to hybridize to cDNAs or genomic DNAs containing sequences complementary thereto or homologous thereto. For probes over 200 nucleotides in length, the hybridization may be carried out at 15-25° C. below the Tm. For shorter probes, such as oligonucleotide probes, the hybridization may be conducted at 5-10° C. below the Tm. In one aspect, hybridizations in 6×SSC are conducted at approximately 68° C. In one aspect, hybridizations in 50% formamide containing solutions are conducted at approximately 42° C. All of the foregoing hybridizations would be considered to be under conditions of high stringency.

Following hybridization, the filter is washed to remove any non-specifically bound detectable probe. The stringency used to wash the filters can also be varied depending on the nature of the nucleic acids being hybridized, the length of the nucleic acids being hybridized, the degree of complementarity, the nucleotide sequence composition (e.g., GC v. AT content), and the nucleic acid type (e.g., RNA v. DNA). Examples of progressively higher stringency condition washes are as follows: 2×SSC, 0.1% SDS at room temperature for 15 minutes (low stringency); 0.1×SSC, 0.5% SDS at room temperature for 30 minutes to 1 hour (moderate stringency); 0.1×SSC, 0.5% SDS for 15 to 30 minutes at between the hybridization temperature and 68° C. (high stringency); and 0.15M NaCl for 15 minutes at 72° C. (very high stringency). A final low stringency wash can be conducted in 0.1×SSC at room temperature. The examples above are merely illustrative of one set of conditions that can be used to wash filters. One of skill in the art would know that there are numerous recipes for different stringency washes.

Nucleic acids which have hybridized to the probe can be identified by autoradiography or other conventional techniques. The above procedure may be modified to identify nucleic acids having decreasing levels of homology to the probe sequence. For example, to obtain nucleic acids of decreasing homology to the detectable probe, less stringent conditions may be used. For example, the hybridization temperature may be decreased in increments of 5° C. from 68° C. to 42° C. in a hybridization buffer having a Na+ concentration of approximately 1M. Following hybridization, the filter may be washed with 2×SSC, 0.5% SDS at the temperature of hybridization. These conditions are considered to be “moderate” conditions above 50° C. and “low” conditions below 50° C. An example of “moderate” hybridization conditions is when the above hybridization is conducted at 55° C. An example of “low stringency” hybridization conditions is when the above hybridization is conducted at 45° C.

Alternatively, the hybridization may be carried out in buffers, such as 6×SSC, containing formamide at a temperature of 42° C. In this case, the concentration of formamide in the hybridization buffer may be reduced in 5% increments from 50% to 0% to identify clones having decreasing levels of homology to the probe. Following hybridization, the filter may be washed with 6×SSC, 0.5% SDS at 50° C. These conditions are considered to be “moderate” conditions above 25% formamide and “low” conditions below 25% formamide. A specific example of “moderate” hybridization conditions is when the above hybridization is conducted at 30% formamide. A specific example of “low stringency” hybridization conditions is when the above hybridization is conducted at 10% formamide.

These probes and methods of the invention can be used to isolate nucleic acids having a sequence with at least about 99%, 98%, 97%, at least 95%, homology to a nucleic acid sequence of the invention comprising at least about 10, 15, 20, 25, 30, 35, 40, 50, 75, 100, 150, 200, 250, 300, 350, 400, 500, 550, 600, 650, 700, 750, 800, 850, 900, 950, 1000, or more consecutive bases thereof, and the sequences complementary thereto. Homology may be measured using an alignment algorithm, as discussed herein. For example, the homologous polynucleotides may have a coding sequence which is a naturally occurring allelic variant of one of the coding sequences described herein. Such allelic variants may have a substitution, deletion or addition of one or more nucleotides when compared to a nucleic acid of the invention.

Additionally, the probes and methods of the invention may be used to isolate nucleic acids which encode polypeptides having at least about 99%, at least 98%, at least 97%, at least 96%, at least 95% sequence identity (homology) to a polypeptide of the invention comprising at least 5, 10, 15, 20, 25, 30, 35, 40, 50, 75, 100, or 150 or more consecutive amino acids thereof as determined using a sequence alignment algorithm (e.g., such as the FASTA version 3.0t78 algorithm with the default parameters, or a BLAST 2.2.2 program with exemplary settings as set forth herein).

Inhibiting Expression of Xylose Isomerases

The invention further provides for nucleic acids complementary to (e.g., antisense sequences to) the nucleic acid sequences of the invention, e.g., xylose isomerase-encoding sequences. Antisense sequences are capable of inhibiting the transport, splicing or transcription of xylose isomerase-encoding genes. The inhibition can be effected through the targeting of genomic DNA or messenger RNA. The transcription or function of targeted nucleic acid can be inhibited, for example, by hybridization and/or cleavage. One particularly useful set of inhibitors provided by the present invention includes oligonucleotides which are able to either bind xylose isomerase gene or message, in either case preventing or inhibiting the production or function of xylose isomerase. The association can be through sequence specific hybridization. Another useful class of inhibitors includes oligonucleotides which cause inactivation or cleavage of xylose isomerase message. The oligonucleotide can have enzyme activity which causes such cleavage, such as ribozymes. The oligonucleotide can be chemically modified or conjugated to an enzyme or composition capable of cleaving the complementary nucleic acid. One may screen a pool of many different such oligonucleotides for those with the desired activity. Thus, the invention provides various compositions for the inhibition of xylose isomerase expression on a nucleic acid and/or protein level, e.g., antisense, iRNA and ribozymes comprising xylose isomerase sequences of the invention and the anti-xylose isomerase antibodies of the invention.

Antisense Oligonucleotides

The invention provides antisense oligonucleotides capable of binding xylose isomerase message which can inhibit isomerase activity by targeting mRNA. Strategies for designing antisense oligonucleotides are well described in the scientific and patent literature, and the skilled artisan can design such xylose isomerase oligonucleotides using the novel reagents of the invention. For example, gene walking/RNA mapping protocols to screen for effective antisense oligonucleotides are well known in the art, see, e.g., Ho (2000) Methods Enzymol. 314:168-183, describing an RNA mapping assay, which is based on standard molecular techniques to provide an easy and reliable method for potent antisense sequence selection. See also Smith (2000) Eur. J. Pharm. Sci. 11: 191-198.

Naturally occurring nucleic acids are used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as described in WO 97/03211; WO 96/39154; Mata (1997) Toxicol Appl Pharmacol 144:189-197; Antisense Therapeutics, ed. Agrawal (Humana Press, Totowa, N.J., 1996). Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense xylose isomerase sequences of the invention (see, e.g., Gold (1995) J. of Biol. Chem. 270:13581-13584).

Inhibitory Ribozymes

The invention provides for with ribozymes capable of binding xylose isomerase message that can inhibit isomerase activity by targeting mRNA. Strategies for designing ribozymes and selecting the xylose isomerase-specific antisense sequence for targeting are well described in the scientific and patent literature, and the skilled artisan can design such ribozymes using the novel reagents of the invention. Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary basepairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it is typically released from that RNA and so can bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The enzymatic ribozyme RNA molecule can be formed in a hammerhead motif, but may also be formed in the motif of a hairpin, hepatitis delta virus, group I intron or RnaseP-like RNA (in association with an RNA guide sequence). Examples of such hammerhead motifs are described by Rossi (1992) Aids Research and Human Retroviruses 8:183; hairpin motifs by Hampel (1989) Biochemistry 28:4929, and Hampel (1990) Nuc. Acids Res. 18:299; the hepatitis delta virus motif by Perrotta (1992) Biochemistry 31:16; the RNaseP motif by Guerrier-Takada (1983) Cell 35:849; and the group I intron by Cech U.S. Pat. No. 4,987,071. The recitation of these specific motifs is not intended to be limiting; those skilled in the art will recognize that an enzymatic RNA molecule of this invention has a specific substrate binding site complementary to one or more of the target gene RNA regions, and has nucleotide sequence within or surrounding that substrate binding site which imparts an RNA cleaving activity to the molecule.

RNA interference (RNAi)

In one aspect, the invention provides an RNA inhibitory molecule, a so-called “RNAi” molecule, comprising a nucleic acid sequence of the invention. The RNAi molecule comprises a double-stranded RNA (dsRNA) molecule. The RNAi can inhibit expression of a xylose isomerase gene. In one aspect, the RNAi is about 15, 16, 17, 18, 19, 20, 21, 22, 23, 24, 25 or more duplex nucleotides in length. While the invention is not limited by any particular mechanism of action, the RNAi can enter a cell and cause the degradation of a single-stranded RNA (ssRNA) of similar or identical sequences, including endogenous mRNAs. When a cell is exposed to double-stranded RNA (dsRNA), mRNA from the homologous gene is selectively degraded by a process called RNA interference (RNAi). A possible basic mechanism behind RNAi is the breaking of a double-stranded RNA (dsRNA) matching a specific gene sequence into short pieces called short interfering RNA, which trigger the degradation of mRNA that matches its sequence. In one aspect, the RNAi's of the invention are used in gene-silencing therapeutics, see, e.g., Shuey (2002) Drug Discov. Today 7:1040-1046. In one aspect, the invention provides methods to selectively degrade RNA using the RNAi's of the invention. The process may be practiced in vitro, ex vivo or in vivo. In one aspect, the RNAi molecules of the invention can be used to generate a loss-of-function mutation in a cell, an organ or an animal. Methods for making and using RNAi molecules for selectively degrade RNA are well known in the art, see, e.g., U.S. Pat. Nos. 6,506,559; 6,511,824; 6,515,109; 6,489,127.

Modification of Nucleic Acids

The invention provides methods of generating variants of the nucleic acids of the invention, e.g., those encoding a xylose isomerase. These methods can be repeated or used in various combinations to generate xylose isomerases having an altered or different activity or an altered or different stability from that of a xylose isomerase encoded by the template nucleic acid. These methods also can be repeated or used in various combinations, e.g., to generate variations in gene/message expression, message translation or message stability. In another aspect, the genetic composition of a cell is altered by, e.g., modification of a homologous gene ex vivo, followed by its reinsertion into the cell.

A nucleic acid of the invention can be altered by any means. For example, random or stochastic methods, or, non-stochastic, or “directed evolution,” methods, see, e.g., U.S. Pat. No. 6,361,974. Methods for random mutation of genes are well known in the art, see, e.g., U.S. Pat. No. 5,830,696. For example, mutagens can be used to randomly mutate a gene. Mutagens include, e.g., ultraviolet light or gamma irradiation, or a chemical mutagen, e.g., mitomycin, nitrous acid, photoactivated psoralens, alone or in combination, to induce DNA breaks amenable to repair by recombination. Other chemical mutagens include, for example, sodium bisulfite, nitrous acid, hydroxylamine, hydrazine or formic acid. Other mutagens are analogues of nucleotide precursors, e.g., nitrosoguanidine, 5-bromouracil, 2-aminopurine, or acridine. These agents can be added to a PCR reaction in place of the nucleotide precursor thereby mutating the sequence. Intercalating agents such as proflavine, acriflavine, quinacrine and the like can also be used.

Any technique in molecular biology can be used, e.g., random PCR mutagenesis, see, e.g., Rice (1992) Proc. Natl. Acad. Sci. USA 89:5467-5471; or, combinatorial multiple cassette mutagenesis, see, e.g., Crameri (1995) Biotechniques 18:194-196. Alternatively, nucleic acids, e.g., genes, can be reassembled after random, or “stochastic,” fragmentation, see, e.g., U.S. Pat. Nos. 6,291,242; 6,287,862; 6,287,861; 5,955,358; 5,830,721; 5,824,514; 5,811,238; 5,605,793. In alternative aspects, modifications, additions or deletions are introduced by error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturated mutagenesis (GSSM™), synthetic ligation reassembly (SLR), recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, and/or a combination of these and other methods.

The following publications describe a variety of recursive recombination procedures and/or methods which can be incorporated into the methods of the invention: Stemmer (1999) “Molecular breeding of viruses for targeting and other clinical properties” Tumor Targeting 4:1-4; Ness (1999) Nature Biotechnology 17:893-896; Chang (1999) “Evolution of a cytokine using DNA family shuffling” Nature Biotechnology 17:793-797; Minshull (1999) “Protein evolution by molecular breeding” Current Opinion in Chemical Biology 3:284-290; Christians (1999) “Directed evolution of thymidine kinase for AZT phosphorylation using DNA family shuffling” Nature Biotechnology 17:259-264; Crameri (1998) “DNA shuffling of a family of genes from diverse species accelerates directed evolution” Nature 391:288-291; Crameri (1997) “Molecular evolution of an arsenate detoxification pathway by DNA shuffling,” Nature Biotechnology 15:436-438; Zhang (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp. 447-457; Crameri and Stemmer (1995) “Combinatorial multiple cassette mutagenesis creates all the permutations of mutant and wildtype cassettes” BioTechniques 18:194-195; Stemmer et al. (1995) “Single-step assembly of a gene and entire plasmid form large numbers of oligodeoxyribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional protocols used in the methods of the invention include point mismatch repair (Kramer (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh (1986) “Use of oligonucleotides to generate large deletions” Nucl. Acids Res. 14: 5115), restriction-selection and restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundstrom et al. (1985) “Oligonucleotide-directed mutagenesis by microscale ‘shot-gun’ gene synthesis” Nucl. Acids Res. 13: 3305-3316), double-strand break repair (Mandecki (1986); Arnold (1993) “Protein engineering for unusual environments” Current Opinion in Biotechnology 4:450-455. “Oligonucleotide-directed double-strand break repair in plasmids of Escherichia coli: a method for site-specific mutagenesis” Proc. Natl. Acad. Sci. USA, 83:7177-7181). Additional details on many of the above methods can be found in Methods in Enzymology Volume 154, which also describes useful controls for trouble-shooting problems with various mutagenesis methods.

Additional protocols used in the methods of the invention include those discussed in U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library ImLmunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination.”

Protocols that can be used to practice the invention (providing details regarding various diversity generating methods) are described, e.g., in U.S. patent application Ser. No. 09/407,800, “SHUFFLING OF CODON ALTERED GENES” by Patten et al. filed Sep. 28, 1999; “EVOLUTION OF WHOLE CELLS AND ORGANISMS BY RECURSIVE SEQUENCE RECOMBINATION” by del Cardayre et al., U.S. Pat. No. 6,379,964; “OLIGONUCLEOTIDE MEDIATED NUCLEIC ACID RECOMBINATION” by Crameri et al., U.S. Pat. Nos. 6,319,714; 6,368,861; 6,376,246; 6,423,542; 6,426,224 and PCT/US00/01203; “USE OF CODON-VARIED OLIGONUCLEOTIDE SYNTHESIS FOR SYNTHETIC SHUFFLING” by Welch et al., U.S. Pat. No. 6,436,675; “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) and, e.g. “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579); “METHODS OF POPULATING DATA STRUCTURES FOR USE IN EVOLUTIONARY SIMULATIONS” by Selifonov and Stemmer, filed Jan. 18, 2000 (PCT/US00/01138); and “SINGLE-STRANDED NUCLEIC ACID TEMPLATE-MEDIATED RECOMBINATION AND NUCLEIC ACID FRAGMENT ISOLATION” by Affholter, filed Sep. 6, 2000 (U.S. Ser. No. 09/656,549); and U.S. Pat. Nos. 6,177,263; 6,153,410.

Non-stochastic, or “directed evolution,” methods include, e.g., saturation mutagenesis (GSSM™), synthetic ligation reassembly (SLR), or a combination thereof are used to modify the nucleic acids of the invention to generate xylose isomerases with new or altered properties (e.g., activity under highly acidic or alkaline conditions, high temperatures, and the like). Polypeptides encoded by the modified nucleic acids can be screened for an activity before testing for proteolytic or other activity. Any testing modality or protocol can be used, e.g., using a capillary array platform. See, e.g., U.S. Pat. Nos. 6,361,974; 6,280,926; 5,939,250.

Saturation Mutagenesis, or, GSSM™

In one aspect of the invention, non-stochastic gene modification, a “directed evolution process,” is used to generate xylose isomerases with new or altered properties. Variations of this method have been termed “gene site-saturation mutagenesis,” “site-saturation mutagenesis,” “saturation mutagenesis” or simply “GSSM™.” It can be used in combination with other mutagenization processes. See, e.g., U.S. Pat. Nos. 6,171,820; 6,238,884. In one aspect, GSSM™ comprises providing a template polynucleotide and a plurality of oligonucleotides, wherein each oligonucleotide comprises a sequence homologous to the template polynucleotide, thereby targeting a specific sequence of the template polynucleotide, and a sequence that is a variant of the homologous gene; generating progeny polynucleotides comprising non-stochastic sequence variations by replicating the template polynucleotide with the oligonucleotides, thereby generating polynucleotides comprising homologous gene sequence variations.

In one aspect, codon primers containing a degenerate N,N,G/T sequence are used to introduce point mutations into a polynucleotide, so as to generate a set of progeny polypeptides in which a full range of single amino acid substitutions is represented at each amino acid position, e.g., an amino acid residue in an enzyme active site or ligand binding site targeted to be modified. These oligonucleotides can comprise a contiguous first homologous sequence, a degenerate N,N,G/T sequence, and, optionally, a second homologous sequence. The downstream progeny translational products from the use of such oligonucleotides include all possible amino acid changes at each amino acid site along the polypeptide, because the degeneracy of the N,N,G/T sequence includes codons for all 20 amino acids. In one aspect, one such degenerate oligonucleotide (comprised of, e.g., one degenerate N,N,G/T cassette) is used for subjecting each original codon in a parental polynucleotide template to a full range of codon substitutions. In another aspect, at least two degenerate cassettes are used—either in the same oligonucleotide or not, for subjecting at least two original codons in a parental polynucleotide template to a full range of codon substitutions. For example, more than one N,N,G/T sequence can be contained in one oligonucleotide to introduce amino acid mutations at more than one site. This plurality of N,N,G/T sequences can be directly contiguous, or separated by one or more additional nucleotide sequence(s). In another aspect, oligonucleotides serviceable for introducing additions and deletions can be used either alone or in combination with the codons containing an N,N,G/T sequence, to introduce any combination or permutation of amino acid additions, deletions, and/or substitutions.

In one aspect, simultaneous mutagenesis of two or more contiguous amino acid positions is done using an oligonucleotide that contains contiguous N,N,G/T triplets, i.e. a degenerate (N,N,G/T)n sequence. In another aspect, degenerate cassettes having less degeneracy than the N,N,G/T sequence are used. For example, it may be desirable in some instances to use (e.g. in an oligonucleotide) a degenerate triplet sequence comprised of only one N, where said N can be in the first second or third position of the triplet. Any other bases including any combinations and permutations thereof can be used in the remaining two positions of the triplet. Alternatively, it may be desirable in some instances to use (e.g. in an oligo) a degenerate N,N,N triplet sequence.

In one aspect, use of degenerate triplets (e.g., N,N,G/T triplets) allows for systematic and easy generation of a full range of possible natural amino acids (for a total of 20 amino acids) into each and every amino acid position in a polypeptide (in alternative aspects, the methods also include generation of less than all possible substitutions per amino acid residue, or codon, position). For example, for a 100 amino acid polypeptide, 2000 distinct species (i.e. 20 possible amino acids per position X 100 amino acid positions) can be generated. Through the use of an oligonucleotide or set of oligonucleotides containing a degenerate N,N,G/T triplet, 32 individual sequences can code for all 20 possible natural amino acids. Thus, in a reaction vessel in which a parental polynucleotide sequence is subjected to saturation mutagenesis using at least one such oligonucleotide, there are generated 32 distinct progeny polynucleotides encoding 20 distinct polypeptides. In contrast, the use of a non-degenerate oligonucleotide in site-directed mutagenesis leads to only one progeny polypeptide product per reaction vessel. Nondegenerate oligonucleotides can optionally be used in combination with degenerate primers disclosed; for example, nondegenerate oligonucleotides can be used to generate specific point mutations in a working polynucleotide. This provides one means to generate specific silent point mutations, point mutations leading to corresponding amino acid changes, and point mutations that cause the generation of stop codons and the corresponding expression of polypeptide fragments.

In one aspect, each saturation mutagenesis reaction vessel contains polynucleotides encoding at least 20 progeny polypeptide (e.g., xylose isomerase) molecules such that all 20 natural amino acids are represented at the one specific amino acid position corresponding to the codon position mutagenized in the parental polynucleotide (other aspects use less than all 20 natural combinations). The 32-fold degenerate progeny polypeptides generated from each saturation mutagenesis reaction vessel can be subjected to clonal amplification (e.g. cloned into a suitable host, e.g., E. coli host, using, e.g., an expression vector) and subjected to expression screening. When an individual progeny polypeptide is identified by screening to display a favorable change in property (when compared to the parental polypeptide, such as increased proteolytic activity under alkaline or acidic conditions), it can be sequenced to identify the correspondingly favorable amino acid substitution contained therein.

In one aspect, upon mutagenizing each and every amino acid position in a parental polypeptide using saturation mutagenesis as disclosed herein, favorable amino acid changes may be identified at more than one amino acid position. One or more new progeny molecules can be generated that contain a combination of all or part of these favorable amino acid substitutions. For example, if 2 specific favorable amino acid changes are identified in each of 3 amino acid positions in a polypeptide, the permutations include 3 possibilities at each position (no change from the original amino acid, and each of two favorable changes) and 3 positions. Thus, there are 3×3×3 or 27 total possibilities, including 7 that were previously examined—6 single point mutations (i.e. 2 at each of three positions) and no change at any position.

In another aspect, site-saturation mutagenesis can be used together with another stochastic or non-stochastic means to vary sequence, e.g., synthetic ligation reassembly (see below), shuffling, chimerization, recombination and other mutagenizing processes and mutagenizing agents. This invention provides for the use of any mutagenizing process(es), including saturation mutagenesis, in an iterative manner.

Synthetic Ligation Reassembly (SLR)

The invention provides a non-stochastic gene modification system termed “synthetic ligation reassembly,” or simply “SLR,” a “directed evolution process,” to generate xylose isomerases with new or altered properties. SLR is a method of ligating oligonucleotide fragments together non-stochastically. This method differs from stochastic oligonucleotide shuffling in that the nucleic acid building blocks are not shuffled, concatenated or chimerized randomly, but rather are assembled non-stochastically. See, e.g., U.S. patent application Ser. No. 09/332,835 entitled “Synthetic Ligation Reassembly in Directed Evolution” and filed on Jun. 14, 1999 (“U.S. Ser. No. 09/332,835”). In one aspect, SLR comprises the following steps: (a) providing a template polynucleotide, wherein the template polynucleotide comprises sequence encoding a homologous gene; (b) providing a plurality of building block polynucleotides, wherein the building block polynucleotides are designed to cross-over reassemble with the template polynucleotide at a predetermined sequence, and a building block polynucleotide comprises a sequence that is a variant of the homologous gene and a sequence homologous to the template polynucleotide flanking the variant sequence; (c) combining a building block polynucleotide with a template polynucleotide such that the building block polynucleotide cross-over reassembles with the template polynucleotide to generate polynucleotides comprising homologous gene sequence variations.

SLR does not depend on the presence of high levels of homology between polynucleotides to be rearranged. Thus, this method can be used to non-stochastically generate libraries (or sets) of progeny molecules comprised of over 10100 different chimeras. SLR can be used to generate libraries comprised of over 101000 different progeny chimeras. Thus, aspects of the present invention include non-stochastic methods of producing a set of finalized chimeric nucleic acid molecule shaving an overall assembly order that is chosen by design. This method includes the steps of generating by design a plurality of specific nucleic acid building blocks having serviceable mutually compatible ligatable ends, and assembling these nucleic acid building blocks, such that a designed overall assembly order is achieved.

The mutually compatible ligatable ends of the nucleic acid building blocks to be assembled are considered to be “serviceable” for this type of ordered assembly if they enable the building blocks to be coupled in predetermined orders. Thus, the overall assembly order in which the nucleic acid building blocks can be coupled is specified by the design of the ligatable ends. If more than one assembly step is to be used, then the overall assembly order in which the nucleic acid building blocks can be coupled is also specified by the sequential order of the assembly step(s). In one aspect, the annealed building pieces are treated with an enzyme, such as a ligase (e.g. T4 DNA ligase), to achieve covalent bonding of the building pieces.

In one aspect, the design of the oligonucleotide building blocks is obtained by analyzing a set of progenitor nucleic acid sequence templates that serve as a basis for producing a progeny set of finalized chimeric polynucleotides. These parental oligonucleotide templates thus serve as a source of sequence information that aids in the design of the nucleic acid building blocks that are to be mutagenized, e.g., chimerized or shuffled. In one aspect of this method, the sequences of a plurality of parental nucleic acid templates are aligned in order to select one or more demarcation points. The demarcation points can be located at an area of homology, and are comprised of one or more nucleotides. These demarcation points can be shared by at least two of the progenitor templates. The demarcation points can thereby be used to delineate the boundaries of oligonucleotide building blocks to be generated in order to rearrange the parental polynucleotides. The demarcation points identified and selected in the progenitor molecules serve as potential chimerization points in the assembly of the final chimeric progeny molecules. A demarcation point can be an area of homology (comprised of at least one homologous nucleotide base) shared by at least two parental polynucleotide sequences. Alternatively, a demarcation point can be an area of homology that is shared by at least half of the parental polynucleotide sequences, or, it can be an area of homology that is shared by at least two thirds of the parental polynucleotide sequences. Alternatively, a serviceable demarcation points is an area of homology that is shared by at least three fourths of the parental polynucleotide sequences, or, it can be shared by at almost all of the parental polynucleotide sequences. In one aspect, a demarcation point is an area of homology that is shared by all of the parental polynucleotide sequences.

In one aspect, a ligation reassembly process is performed exhaustively in order to generate an exhaustive library of progeny chimeric polynucleotides. In other words, all possible ordered combinations of the nucleic acid building blocks are represented in the set of finalized chimeric nucleic acid molecules. At the same time, in another aspect, the assembly order (i.e. the order of assembly of each building block in the 5′ to 3 sequence of each finalized chimeric nucleic acid) in each combination is by design (or non-stochastic) as described above. Because of the non-stochastic nature of this invention, the possibility of unwanted side products is greatly reduced.

In another aspect, the ligation reassembly method is performed systematically. For example, the method is performed in order to generate a systematically compartmentalized library of progeny molecules, with compartments that can be screened systematically, e.g. one by one. In other words this invention provides that, through the selective and judicious use of specific nucleic acid building blocks, coupled with the selective and judicious use of sequentially stepped assembly reactions, a design can be achieved where specific sets of progeny products are made in each of several reaction vessels. This allows a systematic examination and screening procedure to be performed. Thus, these methods allow a potentially very large number of progeny molecules to be examined systematically in smaller groups. Because of its ability to perform chimerizations in a manner that is highly flexible yet exhaustive and systematic as well, particularly when there is a low level of homology among the progenitor molecules, these methods provide for the generation of a library (or set) comprised of a large number of progeny molecules. Because of the non-stochastic nature of the instant ligation reassembly invention, the progeny molecules generated can comprise a library of finalized chimeric nucleic acid molecules having an overall assembly order that is chosen by design. The saturation mutagenesis and optimized directed evolution methods also can be used to generate different progeny molecular species. It is appreciated that the invention provides freedom of choice and control regarding the selection of demarcation points, the size and number of the nucleic acid building blocks, and the size and design of the couplings. It is appreciated, furthermore, that the requirement for intermolecular homology is highly relaxed for the operability of this invention. In fact, demarcation points can even be chosen in areas of little or no intermolecular homology. For example, because of codon wobble, i.e. the degeneracy of codons, nucleotide substitutions can be introduced into nucleic acid building blocks without altering the amino acid originally encoded in the corresponding progenitor template. Alternatively, a codon can be altered such that the coding for an originally amino acid is altered. This invention provides that such substitutions can be introduced into the nucleic acid building block in order to increase the incidence of intermolecular homologous demarcation points and thus to allow an increased number of couplings to be achieved among the building blocks, which in turn allows a greater number of progeny chimeric molecules to be generated.

In another aspect, the synthetic nature of the step in which the building blocks are generated allows the design and introduction of nucleotides (e.g., one or more nucleotides, which may be, for example, codons or introns or regulatory sequences) that can later be optionally removed in an in vitro process (e.g. by mutagenesis) or in an in vivo process (e.g. by utilizing the gene splicing ability of a host organism). It is appreciated that in many instances the introduction of these nucleotides may also be desirable for many other reasons in addition to the potential benefit of creating a serviceable demarcation point.

In one aspect, a nucleic acid building block is used to introduce an intron. Thus, functional introns are introduced into a man-made gene manufactured according to the methods described herein. The artificially introduced intron(s) can be functional in a host cells for gene splicing much in the way that naturally-occurring introns serve functionally in gene splicing.

Optimized Directed Evolution System

The invention provides a non-stochastic gene modification system termed “optimized directed evolution system” to generate xylose isomerases with new or altered properties. Optimized directed evolution is directed to the use of repeated cycles of reductive reassortment, recombination and selection that allow for the directed molecular evolution of nucleic acids through recombination. Optimized directed evolution allows generation of a large population of evolved chimeric sequences, wherein the generated population is significantly enriched for sequences that have a predetermined number of crossover events.

A crossover event is a point in a chimeric sequence where a shift in sequence occurs from one parental variant to another parental variant. Such a point is normally at the juncture of where oligonucleotides from two parents are ligated together to form a single sequence. This method allows calculation of the correct concentrations of oligonucleotide sequences so that the final chimeric population of sequences is enriched for the chosen number of crossover events. This provides more control over choosing chimeric variants having a predetermined number of crossover events.

In addition, this method provides a convenient means for exploring a tremendous amount of the possible protein variant space in comparison to other systems. Previously, if one generated, for example, 1013 chimeric molecules during a reaction, it would be extremely difficult to test such a high number of chimeric variants for a particular activity. Moreover, a significant portion of the progeny population would have a very high number of crossover events which resulted in proteins that were less likely to have increased levels of a particular activity. By using these methods, the population of chimerics molecules can be enriched for those variants that have a particular number of crossover events. Thus, although one can still generate 1013 chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

One method for creating a chimeric progeny polynucleotide sequence is to create oligonucleotides corresponding to fragments or portions of each parental sequence. Each oligonucleotide can includes a unique region of overlap so that mixing the oligonucleotides together results in a new variant that has each oligonucleotide fragment assembled in the correct order. Additional information can also be found, e.g., in U.S. Ser. No. 09/332,835; U.S. Pat. No. 6,361,974. The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a ⅓ chance (assuming 3 parents) that an oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. By utilizing these methods, one can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events. These methods are directed to the use of repeated cycles of reductive reassortment, recombination and selection that allow for the directed molecular evolution of a nucleic acid encoding a polypeptide through recombination. This system allows generation of a large population of evolved chimeric sequences, wherein the generated population is significantly enriched for sequences that have a predetermined number of crossover events. A crossover event is a point in a chimeric sequence where a shift in sequence occurs from one parental variant to another parental variant. Such a point is normally at the juncture of where oligonucleotides from two parents are ligated together to form a single sequence. The method allows calculation of the correct concentrations of oligonucleotide sequences so that the final chimeric population of sequences is enriched for the chosen number of crossover events. This provides more control over choosing chimeric variants having a predetermined number of crossover events.

In addition, these methods provide a convenient means for exploring a tremendous amount of the possible protein variant space in comparison to other systems. By using the methods described herein, the population of chimerics molecules can be enriched for those variants that have a particular number of crossover events. Thus, although one can still generate 1013 chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

In one aspect, the method creates a chimeric progeny polynucleotide sequence by creating oligonucleotides corresponding to fragments or portions of each parental sequence. Each oligonucleotide can includes a unique region of overlap so that mixing the oligonucleotides together results in a new variant that has each oligonucleotide fragment assembled in the correct order. See also U.S. Pat. Nos. 6,537,776; 6,605,449.

The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a ⅓ chance (assuming 3 parents) that an oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. One can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events.

Determining Crossover Events

Aspects of the invention include a system and software that receive a desired crossover probability density function (PDF), the number of parent genes to be reassembled, and the number of fragments in the reassembly as inputs. The output of this program is a “fragment PDF” that can be used to determine a recipe for producing reassembled genes, and the estimated crossover PDF of those genes. The processing described herein is can be performed in MATLAB™ (The Mathworks, Natick, Mass.) a programming language and development environment for technical computing.

Iterative Processes

In practicing the invention, these processes can be iteratively repeated. For example a nucleic acid (or, the nucleic acid) responsible for an altered xylose isomerase phenotype is identified, re-isolated, again modified, re-tested for activity. This process can be iteratively repeated until a desired phenotype is engineered. For example, an entire biochemical anabolic or catabolic pathway can be engineered into a cell, including proteolytic activity.

Similarly, if it is determined that a particular oligonucleotide has no affect at all on the desired trait (e.g., a new xylose isomerase phenotype), it can be removed as a variable by synthesizing larger parental oligonucleotides that include the sequence to be removed. Since incorporating the sequence within a larger sequence prevents any crossover events, there will no longer be any variation of this sequence in the progeny polynucleotides. This iterative practice of determining which oligonucleotides are most related to the desired trait, and which are unrelated, allows more efficient exploration all of the possible protein variants that might be provide a particular trait or activity.

In Vivo Shuffling

In vivo shuffling of molecules is use in methods of the invention that provide variants of polypeptides of the invention, e.g., antibodies, xylose isomerases, and the like. In vivo shuffling can be performed utilizing the natural property of cells to recombine multimers. While recombination in vivo has provided the major natural route to molecular diversity, genetic recombination remains a relatively complex process that involves 1) the recognition of homologies; 2) strand cleavage, strand invasion, and metabolic steps leading to the production of recombinant chiasma; and finally 3) the resolution of chiasma into discrete recombined molecules. The formation of the chiasma requires the recognition of homologous sequences.

In one aspect, the invention provides a method for producing a hybrid polynucleotide from at least a first polynucleotide and a second polynucleotide. The invention can be used to produce a hybrid polynucleotide by introducing at least a first polynucleotide and a second polynucleotide which share at least one region of partial sequence homology into a suitable host cell. The regions of partial sequence homology promote processes which result in sequence reorganization producing a hybrid polynucleotide. The term “hybrid polynucleotide”, as used herein, is any nucleotide sequence which results from the method of the present invention and contains sequence from at least two original polynucleotide sequences. Such hybrid polynucleotides can result from intermolecular recombination events which promote sequence integration between DNA molecules. In addition, such hybrid polynucleotides can result from intramolecular reductive reassortment processes which utilize repeated sequences to alter a nucleotide sequence within a DNA molecule.

Producing Sequence Variants

The invention also provides methods of making sequence variants of the nucleic acid and xylose isomerase sequences of the invention or isolating xylose isomerase using the nucleic acids and polypeptides of the invention. In one aspect, the invention provides for variants of a xylose isomerase gene of the invention, which can be altered by any means, including, e.g., random or stochastic methods, or, non-stochastic, or “directed evolution,” methods, as described above.

The isolated variants may be naturally occurring. Variant can also be created in vitro. Variants may be created using genetic engineering techniques such as site directed mutagenesis, random chemical mutagenesis, Exonuclease III deletion procedures, and standard cloning techniques. Alternatively, such variants, fragments, analogs, or derivatives may be created using chemical synthesis or modification procedures. Other methods of making variants are also familiar to those skilled in the art. These include procedures in which nucleic acid sequences obtained from natural isolates are modified to generate nucleic acids which encode polypeptides having characteristics which enhance their value in industrial or laboratory applications. In such procedures, a large number of variant sequences having one or more nucleotide differences with respect to the sequence obtained from the natural isolate are generated and characterized. These nucleotide differences can result in amino acid changes with respect to the polypeptides encoded by the nucleic acids from the natural isolates.

For example, variants may be created using error prone PCR. In error prone PCR, PCR is performed under conditions where the copying fidelity of the DNA polymerase is low, such that a high rate of point mutations is obtained along the entire length of the PCR product. Error prone PCR is described, e.g., in Leung (1989) Technique 1:11-15) and Caldwell (1992) PCR Methods Applic. 2:28-33. Briefly, in such procedures, nucleic acids to be mutagenized are mixed with PCR primers, reaction buffer, MgCl₂, MnCl₂, Taq polymerase and an appropriate concentration of dNTPs for achieving a high rate of point mutation along the entire length of the PCR product. For example, the reaction may be performed using 20 fmoles of nucleic acid to be mutagenized, 30 pmole of each PCR primer, a reaction buffer comprising 50 mM KCl, 10 mM Tris HCl (pH 8.3) and 0.01% gelatin, 7 mM MgCl₂, 0.5 mM MnCl₂, 5 units of Taq polymerase, 0.2 mM dGTP, 0.2 mM dATP, 1 mM dCTP, and 1 mM dTTP. PCR may be performed for 30 cycles of 94° C. for 1 min, 45° C. for 1 min, and 72° C. for 1 min. However, it will be appreciated that these parameters may be varied as appropriate. The mutagenized nucleic acids are cloned into an appropriate vector and the activities of the polypeptides encoded by the mutagenized nucleic acids is evaluated.

Variants may also be created using oligonucleotide directed mutagenesis to generate site-specific mutations in any cloned DNA of interest. Oligonucleotide mutagenesis is described, e.g., in Reidhaar-Olson (1988) Science 241:53-57. Briefly, in such procedures a plurality of double stranded oligonucleotides bearing one or more mutations to be introduced into the cloned DNA are synthesized and inserted into the cloned DNA to be mutagenized. Clones containing the mutagenized DNA are recovered and the activities of the polypeptides they encode are assessed.

Another method for generating variants is assembly PCR. Assembly PCR involves the assembly of a PCR product from a mixture of small DNA fragments. A large number of different PCR reactions occur in parallel in the same vial, with the products of one reaction priming the products of another reaction. Assembly PCR is described in, e.g., U.S. Pat. No. 5,965,408.

Still another method of generating variants is sexual PCR mutagenesis. In sexual PCR mutagenesis, forced homologous recombination occurs between DNA molecules of different but highly related DNA sequence in vitro, as a result of random fragmentation of the DNA molecule based on sequence homology, followed by fixation of the crossover by primer extension in a PCR reaction. Sexual PCR mutagenesis is described, e.g., in Stemmer (1994) Proc. Natl. Acad. Sci. USA 91:10747-10751. Briefly, in such procedures a plurality of nucleic acids to be recombined are digested with DNase to generate fragments having an average size of 50-200 nucleotides. Fragments of the desired average size are purified and resuspended in a PCR mixture. PCR is conducted under conditions which facilitate recombination between the nucleic acid fragments. For example, PCR may be performed by resuspending the purified fragments at a concentration of 10-30 ng/:1 in a solution of 0.2 mM of each dNTP, 2.2 mM MgCl₂, 50 mM KCL, 10 mM Tris HCl, pH 9.0, and 0.1% Triton X-100. 2.5 units of Taq polymerase per 100:1 of reaction mixture is added and PCR is performed using the following regime: 94° C. for 60 seconds, 94° C. for 30 seconds, 50-55° C. for 30 seconds, 72° C. for 30 seconds (30-45 times) and 72° C. for 5 minutes. However, it will be appreciated that these parameters may be varied as appropriate. In some aspects, oligonucleotides may be included in the PCR reactions. In other aspects, the Klenow fragment of DNA polymerase I may be used in a first set of PCR reactions and Taq polymerase may be used in a subsequent set of PCR reactions. Recombinant sequences are isolated and the activities of the polypeptides they encode are assessed.

Variants may also be created by in vivo mutagenesis. In some aspects, random mutations in a sequence of interest are generated by propagating the sequence of interest in a bacterial strain, such as an E. coli strain, which carries mutations in one or more of the DNA repair pathways. Such “mutator” strains have a higher random mutation rate than that of a wild-type parent. Propagating the DNA in one of these strains will eventually generate random mutations within the DNA. Mutator strains suitable for use for in vivo mutagenesis are described, e.g., in PCT Publication No. WO 91/16427.

Variants may also be generated using cassette mutagenesis. In cassette mutagenesis a small region of a double stranded DNA molecule is replaced with a synthetic oligonucleotide “cassette” that differs from the native sequence. The oligonucleotide often contains completely and/or partially randomized native sequence.

Recursive ensemble mutagenesis may also be used to generate variants. Recursive ensemble mutagenesis is an algorithm for protein engineering (protein mutagenesis) developed to produce diverse populations of phenotypically related mutants whose members differ in amino acid sequence. This method uses a feedback mechanism to control successive rounds of combinatorial cassette mutagenesis. Recursive ensemble mutagenesis is described, e.g., in Arkin (1992) Proc. Natl. Acad. Sci. USA 89:7811-7815.

In some aspects, variants are created using exponential ensemble mutagenesis. Exponential ensemble mutagenesis is a process for generating combinatorial libraries with a high percentage of unique and functional mutants, wherein small groups of residues are randomized in parallel to identify, at each altered position, amino acids which lead to functional proteins. Exponential ensemble mutagenesis is described, e.g., in Delegrave (1993) Biotechnology Res. 11:1548-1552. Random and site-directed mutagenesis are described, e.g., in Arnold (1993) Current Opinion in Biotechnology 4:450-455.

In some aspects, the variants are created using shuffling procedures wherein portions of a plurality of nucleic acids which encode distinct polypeptides are fused together to create chimeric nucleic acid sequences which encode chimeric polypeptides as described in, e.g., U.S. Pat. Nos. 5,965,408; 5,939,250.

The invention also provides variants of polypeptides of the invention comprising sequences in which one or more of the amino acid residues (e.g., of an exemplary polypeptide, such as SEQ ID NO:2, SEQ ID NO:4, SEQ ID NO:6) are substituted with a conserved or non-conserved amino acid residue (e.g., a conserved amino acid residue) and such substituted amino acid residue may or may not be one encoded by the genetic code. Conservative substitutions are those that substitute a given amino acid in a polypeptide by another amino acid of like characteristics. Thus, polypeptides of the invention include those with conservative substitutions of sequences of the invention, e.g., the exemplary SEQ ID NO:2, SEQ ID NO:4, SEQ ID NO:6, including but not limited to the following replacements: replacements of an aliphatic amino acid such as Alanine, Valine, Leucine and Isoleucine with another aliphatic amino acid; replacement of a Serine with a Threonine or vice versa; replacement of an acidic residue such as Aspartic acid and Glutamic acid with another acidic residue; replacement of a residue bearing an amide group, such as Asparagine and Glutamine, with another residue bearing an amide group; exchange of a basic residue such as Lysine and Arginine with another basic residue; and replacement of an aromatic residue such as Phenylalanine, Tyrosine with another aromatic residue. Other variants are those in which one or more of the amino acid residues of the polypeptides of the invention includes a substituent group.

Other variants within the scope of the invention are those in which the polypeptide is associated with another compound, such as a compound to increase the half-life of the polypeptide, for example, polyethylene glycol.

Additional variants within the scope of the invention are those in which additional amino acids are fused to the polypeptide, such as a leader sequence, a secretory sequence, a proprotein sequence or a sequence which facilitates purification, enrichment, or stabilization of the polypeptide.

In some aspects, the variants, fragments, derivatives and analogs of the polypeptides of the invention retain the same biological function or activity as the exemplary polypeptides, e.g., a proteolytic activity, as described herein. In other aspects, the variant, fragment, derivative, or analog includes a proprotein, such that the variant, fragment, derivative, or analog can be activated by cleavage of the proprotein portion to produce an active polypeptide.

Optimizing Codons to Achieve High Levels of Protein Expression in Host Cells

The invention provides methods for modifying xylose isomerase-encoding nucleic acids to modify codon usage. In one aspect, the invention provides methods for modifying codons in a nucleic acid encoding a xylose isomerase to increase or decrease its expression in a host cell, e.g., a bacterial, insect, mammalian, yeast or plant cell. The invention also provides nucleic acids encoding a xylose isomerase modified to increase its expression in a host cell, xylose isomerase so modified, and methods of making the modified xylose isomerases. The method comprises identifying a “non-preferred” or a “less preferred” codon in xylose isomerase-encoding nucleic acid and replacing one or more of these non-preferred or less preferred codons with a “preferred codon” encoding the same amino acid as the replaced codon and at least one non-preferred or less preferred codon in the nucleic acid has been replaced by a preferred codon encoding the same amino acid. A preferred codon is a codon over-represented in coding sequences in genes in the host cell and a non-preferred or less preferred codon is a codon under-represented in coding sequences in genes in the host cell.

Host cells for expressing the nucleic acids, expression cassettes and vectors of the invention include bacteria, yeast, fungi, plant cells, insect cells and mammalian cells. Thus, the invention provides methods for optimizing codon usage in all of these cells, codon-altered nucleic acids and polypeptides made by the codon-altered nucleic acids. Exemplary host cells include gram negative bacteria, such as Escherichia coli and Pseudomonas fluorescens; gram positive bacteria, such as Streptomyces diversa, Lactobacillus gasseri, Lactococcus lactis, Lactococcus cremoris, Bacillus subtilis. Exemplary host cells also include eukaryotic organisms, e.g., various yeast, such as Saccharomyces sp., including Saccharomyces cerevisiae, Schizosaccharomyces pombe, Pichia pastoris, and Kluyveromyces lactis, Hansenula polymorpha, Aspergillus niger, and mammalian cells and cell lines and insect cells and cell lines. Thus, the invention also includes nucleic acids and polypeptides optimized for expression in these organisms and species.

For example, the codons of a nucleic acid encoding a xylose isomerase isolated from a bacterial cell are modified such that the nucleic acid is optimally expressed in a bacterial cell different from the bacteria from which the xylose isomerase was derived, a yeast, a fungi, a plant cell, an insect cell or a mammalian cell. Methods for optimizing codons are well known in the art, see, e.g., U.S. Pat. No. 5,795,737; Baca (2000) Int. J. Parasitol. 30:113-118; Hale (1998) Protein Expr. Purif. 12:185-188; Narum (2001) Infect. Immun. 69:7250-7253. See also Narum (2001) Infect. Immun. 69:7250-7253, describing optimizing codons in mouse systems; Outchkourov (2002) Protein Expr. Purif. 24:18-24, describing optimizing codons in yeast; Feng (2000) Biochemistry 39:15399-15409, describing optimizing codons in E. coli; Humphreys (2000) Protein Expr. Purif. 20:252-264, describing optimizing codon usage that affects secretion in E. coli.

Transgenic Non-Human Animals

The invention provides transgenic non-human animals comprising a nucleic acid, a polypeptide, an expression cassette or vector or a transfected or transformed cell of the invention. The transgenic non-human animals can be, e.g., goats, rabbits, sheep, pigs, cows, rats and mice, comprising the nucleic acids of the invention. These animals can be used, e.g., as in vivo models to study xylose isomerase activity, or, as models to screen for agents that change the xylose isomerase activity in vivo. The coding sequences for the polypeptides to be expressed in the transgenic non-human animals can be designed to be constitutive, or, under the control of tissue-specific, developmental-specific or inducible transcriptional regulatory factors. Transgenic non-human animals can be designed and generated using any method known in the art; see, e.g., U.S. Pat. Nos. 6,211,428; 6,187,992; 6,156,952; 6,118,044; 6,111,166; 6,107,541; 5,959,171; 5,922,854; 5,892,070; 5,880,327; 5,891,698; 5,639,940; 5,573,933; 5,387,742; 5,087,571, describing making and using transformed cells and eggs and transgenic mice, rats, rabbits, sheep, pigs and cows. See also, e.g., Pollock (1999) J. Immunol. Methods 231:147-157, describing the production of recombinant proteins in the milk of transgenic dairy animals; Baguisi (1999) Nat. Biotechnol. 17:456-461, demonstrating the production of transgenic goats. U.S. Pat. No. 6,211,428, describes making and using transgenic non-human mammals which express in their brains a nucleic acid construct comprising a DNA sequence. U.S. Pat. No. 5,387,742, describes injecting cloned recombinant or synthetic DNA sequences into fertilized mouse eggs, implanting the injected eggs in pseudo-pregnant females, and growing to term transgenic mice whose cells express proteins related to the pathology of Alzheimer's disease. U.S. Pat. No. 6,187,992, describes making and using a transgenic mouse whose genome comprises a disruption of the gene encoding amyloid precursor protein (APP).

“Knockout animals” can also be used to practice the methods of the invention. For example, in one aspect, the transgenic or modified animals of the invention comprise a “knockout animal,” e.g., a “knockout mouse,” engineered not to express an endogenous gene, which is replaced with a gene expressing a xylose isomerase of the invention, or, a fusion protein comprising a xylose isomerase of the invention.

Transgenic Plants and Seeds

The invention provides transgenic plants and seeds comprising a nucleic acid, a polypeptide, an expression cassette or vector or a transfected or transformed cell of the invention. The transgenic plant can be dicotyledonous (a dicot) or monocotyledonous (a monocot). The invention also provides methods of making and using these transgenic plants and seeds. The transgenic plant or plant cell expressing a polypeptide of the present invention may be constructed in accordance with any method known in the art. See, for example, U.S. Pat. No. 6,309,872.

Nucleic acids and expression constructs of the invention can be introduced into a plant cell by any means. For example, nucleic acids or expression constructs can be introduced into the genome of a desired plant host, or, the nucleic acids or expression constructs can be episomes. Introduction into the genome of a desired plant can be such that the host's xylose isomerase production is regulated by endogenous transcriptional or translational control elements. The invention also provides “knockout plants” where insertion of gene sequence by, e.g., homologous recombination, has disrupted the expression of the endogenous gene. Means to generate “knockout” plants are well-known in the art, see, e.g., Strepp (1998) Proc Natl. Acad. Sci. USA 95:4368-4373; Miao (1995) Plant J 7:359-365. See discussion on transgenic plants, below.

The nucleic acids of the invention can be used to confer desired traits on essentially any plant, e.g., on glucose or starch-producing plants, such as corn, potato, wheat, rice, barley, and the like. Nucleic acids of the invention can be used to manipulate metabolic pathways of a plant in order to optimize or alter host's expression of xylose isomerase. The can change the ratio of starch/sugar conversion in a plant. This can facilitate industrial processing of a plant. Alternatively, xylose isomerases of the invention can be used in production of a transgenic plant to produce a compound not naturally produced by that plant. This can lower production costs or create a novel product.

In one aspect, the first step in production of a transgenic plant involves making an expression construct for expression in a plant cell. These techniques are well known in the art. They can include selecting and cloning a promoter, a coding sequence for facilitating efficient binding of ribosomes to mRNA and selecting the appropriate gene terminator sequences. One exemplary constitutive promoter is CaMV35S, from the cauliflower mosaic virus, which generally results in a high degree of expression in plants. Other promoters are more specific and respond to cues in the plant's internal or external environment. An exemplary light-inducible promoter is the promoter from the cab gene, encoding the major chlorophyll a/b binding protein.

In one aspect, the nucleic acid is modified to achieve greater expression in a plant cell. For example, a sequence of the invention is likely to have a higher percentage of A-T nucleotide pairs compared to that seen in a plant, some of which prefer G-C nucleotide pairs. Therefore, A-T nucleotides in the coding sequence can be substituted with G-C nucleotides without significantly changing the amino acid sequence to enhance production of the gene product in plant cells.

Selectable marker gene can be added to the gene construct in order to identify plant cells or tissues that have successfully integrated the transgene. This may be necessary because achieving incorporation and expression of genes in plant cells is a rare event, occurring in just a few percent of the targeted tissues or cells. Selectable marker genes encode proteins that provide resistance to agents that are normally toxic to plants, such as antibiotics or herbicides. Only plant cells that have integrated the selectable marker gene will survive when grown on a medium containing the appropriate antibiotic or herbicide. As for other inserted genes, marker genes also require promoter and termination sequences for proper function.

In one aspect, making transgenic plants or seeds comprises incorporating sequences of the invention and, optionally, marker genes into a target expression construct (e.g., a plasmid), along with positioning of the promoter and the terminator sequences. This can involve transferring the modified gene into the plant through a suitable method. For example, a construct may be introduced directly into the genomic DNA of the plant cell using techniques such as electroporation and microinjection of plant cell protoplasts, or the constructs can be introduced directly to plant tissue using ballistic methods, such as DNA particle bombardment. For example, see, e.g., Christou (1997) Plant Mol. Biol. 35:197-203; Pawlowski (1996) Mol. Biotechnol. 6:17-30; Klein (1987) Nature 327:70-73; Takumi (1997) Genes Genet. Syst. 72:63-69, discussing use of particle bombardment to introduce transgenes into wheat; and Adam (1997) supra, for use of particle bombardment to introduce YACs into plant cells. For example, Rinehart (1997) supra, used particle bombardment to generate transgenic cotton plants. Apparatus for accelerating particles is described U.S. Pat. No. 5,015,580; and, the commercially available BioRad (Biolistics) PDS-2000 particle acceleration instrument; see also, John, U.S. Pat. No. 5,608,148; and Ellis, U.S. Pat. No. 5,681,730, describing particle-mediated transformation of gymnosperms.

In one aspect, protoplasts can be immobilized and injected with a nucleic acids, e.g., an expression construct. Although plant regeneration from protoplasts is not easy with cereals, plant regeneration is possible in legumes using somatic embryogenesis from protoplast derived callus. Organized tissues can be transformed with naked DNA using gene gun technique, where DNA is coated on tungsten microprojectiles, shot 1/100th the size of cells, which carry the DNA deep into cells and organelles. Transformed tissue is then induced to regenerate, usually by somatic embryogenesis. This technique has been successful in several cereal species including maize and rice.

Nucleic acids, e.g., expression constructs, can also be introduced in to plant cells using recombinant viruses. Plant cells can be transformed using viral vectors, such as, e.g., tobacco mosaic virus derived vectors (Rouwendal (1997) Plant Mol. Biol. 33:989-999), see Porta (1996) “Use of viral replicons for the expression of genes in plants,” Mol. Biotechnol. 5:209-221.

Alternatively, nucleic acids, e.g., an expression construct, can be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria Agrobacterium tumefaciens-mediated transformation techniques, including disarming and use of binary vectors, are well described in the scientific literature. See, e.g., Horsch (1984) Science 233:496-498; Fraley (1983) Proc. Natl. Acad. Sci. USA 80:4803 (1983); Gene Transfer to Plants, Potrykus, ed. (Springer-Verlag, Berlin 1995). The DNA in an A. tumefaciens cell is contained in the bacterial chromosome as well as in another structure known as a Ti (tumor-inducing) plasmid. The Ti plasmid contains a stretch of DNA termed T-DNA (˜20 kb long) that is transferred to the plant cell in the infection process and a series of vir (virulence) genes that direct the infection process. A. tumefaciens can only infect a plant through wounds: when a plant root or stem is wounded it gives off certain chemical signals, in response to which, the vir genes of A. tumefaciens become activated and direct a series of events necessary for the transfer of the T-DNA from the Ti plasmid to the plant's chromosome. The T-DNA then enters the plant cell through the wound. One speculation is that the T-DNA waits until the plant DNA is being replicated or transcribed, then inserts itself into the exposed plant DNA. In order to use A. tumefaciens as a transgene vector, the tumor-inducing section of T-DNA have to be removed, while retaining the T-DNA border regions and the vir genes. The transgene is then inserted between the T-DNA border regions, where it is transferred to the plant cell and becomes integrated into the plant's chromosomes.

The invention provides for the transformation of monocotyledonous plants using the nucleic acids of the invention, including important cereals, see Hiei (1997) Plant Mol. Biol. 35:205-218. See also, e.g., Horsch, Science (1984) 233:496; Fraley (1983) Proc. Natl. Acad. Sci USA 80:4803; Thykjaer (1997) supra; Park (1996) Plant Mol. Biol. 32:1135-1148, discussing T-DNA integration into genomic DNA. See also D'Halluin, U.S. Pat. No. 5,712,135, describing a process for the stable integration of a DNA comprising a gene that is functional in a cell of a cereal, or other monocotyledonous plant.

In one aspect, the third step can involve selection and regeneration of whole plants capable of transmitting the incorporated target gene to the next generation. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee (1987) Ann. Rev. of Plant Phys. 38:467-486. To obtain whole plants from transgenic tissues such as immature embryos, they can be grown under controlled environmental conditions in a series of media containing nutrients and hormones, a process known as tissue culture. Once whole plants are generated and produce seed, evaluation of the progeny begins.

After the expression cassette is stably incorporated in transgenic plants, it can be introduced into other plants by sexual crossing. Any of a number of standard breeding techniques can be used, depending upon the species to be crossed. Since transgenic expression of the nucleic acids of the invention leads to phenotypic changes, plants comprising the recombinant nucleic acids of the invention can be sexually crossed with a second plant to obtain a final product. Thus, the seed of the invention can be derived from a cross between two transgenic plants of the invention, or a cross between a plant of the invention and another plant. The desired effects (e.g., expression of the polypeptides of the invention to produce a plant in which flowering behavior is altered) can be enhanced when both parental plants express the polypeptides of the invention. The desired effects can be passed to future plant generations by standard propagation means.

The nucleic acids and polypeptides of the invention are expressed in or inserted in any plant or seed. Transgenic plants of the invention can be dicotyledonous or monocotyledonous. Examples of monocot transgenic plants of the invention are grasses, such as meadow grass (blue grass, Poa), forage grass such as festuca, lolium, temperate grass, such as Agrostis, and cereals, e.g., wheat, oats, rye, barley, rice, sorghum, and maize (corn). Examples of dicot transgenic plants of the invention are tobacco, legumes, such as lupins, potato, sugar beet, pea, bean and soybean, and cruciferous plants (family Brassicaceae), such as cauliflower, rape seed, and the closely related model organism Arabidopsis thaliana. Thus, the transgenic plants and seeds of the invention include a broad range of plants, including, but not limited to, species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, and Zea.

In alternative embodiments, the nucleic acids of the invention are expressed in plants which contain fiber cells, including, e.g., cotton, silk cotton tree (Kapok, Ceiba pentandra), desert willow, creosote bush, winterfat, balsa, ramie, kenaf, hemp, roselle, jute, sisal abaca and flax. In alternative embodiments, the transgenic plants of the invention can be members of the genus Gossypium, including members of any Gossypium species, such as G. arboreum; G. herbaceum, G. barbadense, and G. hirsutum.

The invention also provides for transgenic plants to be used for producing large amounts of the polypeptides (e.g., antibodies, xylose isomerases) of the invention. For example, see Palmgren (1997) Trends Genet. 13:348; Chong (1997) Transgenic Res. 6:289-296 (producing human milk protein beta-casein in transgenic potato plants using an auxin-inducible, bidirectional mannopine synthase (mas1′,2′) promoter with Agrobacterium tumefaciens-mediated leaf disc transformation methods).

Using known procedures, one of skill can screen for plants of the invention by detecting the increase or decrease of transgene mRNA or protein in transgenic plants. Means for detecting and quantitation of mRNAs or proteins are well known in the art.

Polypeptides and Peptides

In one aspect, the invention provides isolated or recombinant polypeptides having a sequence identity (e.g., at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity) to an exemplary polypeptide (amino acid) sequence of the invention, e.g., proteins having a sequence as set forth in SEQ ID NO:2; SEQ ID NO:4; SEQ ID NO:6. In one aspect, the identity can be over the full length of the polypeptide, or, the identity can be over a region of at least about 5, 10, 15, 20, 25, 30, 35, 40, 45, 50, 55, 60, 65, 70, 75, 80, 85, 90, 100, 125, 150, 175, 200, 250, 300, 350, 400, 450, 500, 550, 600, 650, 700 or more residues.

Polypeptides of the invention can also be shorter than the full length of exemplary polypeptides (e.g., SEQ ID NO:2; SEQ ID NO:4; SEQ ID NO:6). In alternative aspects, the invention provides polypeptides (peptides, fragments) ranging in size between about 5 and the full length of a polypeptide, e.g., an enzyme, such as a xylose isomerase; exemplary sizes being of about 5, 10, 15, 20, 25, 30, 35, 40, 45, 50, 55, 60, 65, 70, 75, 80, 85, 90, 100, 125, 150, 175, 200, 250, 300, 350, 400, 450, 500, 550, 600, 650, 700, or more residues, e.g., contiguous residues of an exemplary xylose isomerase of the invention. Peptides of the invention can be useful as, e.g., labeling probes, antigens, toleragens, motifs, xylose isomerase active sites. Polypeptides of the invention also include antibodies capable of binding to a xylose isomerase of the invention.

The polypeptides of the invention include xylose isomerases in an active or inactive form. For example, the polypeptides of the invention include proproteins before “maturation” or processing of prepro sequences, e.g., by a proprotein-processing enzyme, such as a proprotein convertase to generate an “active” mature protein. The polypeptides of the invention include xylose isomerases inactive for other reasons, e.g., before “activation” by a post-translational processing event, e.g., an endo- or exo-peptidase or proteinase action, a phosphorylation event, an amidation, a glycosylation or a sulfation, a dimerization event, and the like.

The polypeptides of the invention include all active forms, including active subsequences, e.g., catalytic domains or active sites, of the xylose isomerases. In one aspect, the invention provides a peptide or polypeptide comprising or consisting of an active site domain as predicted through use of a database, e.g., Pfam (Washington Univ., St. Louis, Mo.), which is a large collection of multiple sequence alignments and hidden Markov models covering many common protein families, The Pfam protein families database, A. Bateman, E. Birney, L. Cerruti, R. Durbin, L. Etwiller, S. R. Eddy, S. Griffiths-Jones, K. L. Howe, M. Marshall, and E. L. L. Sonnhammer, Nucleic Acids Research, 30(1):276-280, 2002.

Methods for identifying “prepro” domain sequences and signal sequences are well known in the art, see, e.g., Van de Ven (1993) Crit. Rev. Oncog. 4(2):115-136. For example, to identify a prepro sequence, the protein is purified from the extracellular space and the N-terminal protein sequence is determined and compared to the unprocessed form.

In one aspect, the invention includes polypeptides with or without a signal sequence and/or a prepro sequence. The invention includes polypeptides with heterologous signal sequences and/or prepro sequences. The prepro sequence (including a sequence of the invention used as a heterologous prepro domain) can be located on the amino terminal or the carboxy terminal end of the protein. The invention also includes isolated or recombinant signal sequences, prepro sequences and catalytic domains (e.g., “active sites”) comprising sequences of the invention.

Peptides of the invention (e.g., a subsequence of an exemplary polypeptide of the invention) can be useful as, e.g., labeling probes, antigens, toleragens, motifs, enzyme (e.g., xylose isomerase) active sites (e.g., “catalytic domains”), signal sequences and/or prepro domains.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer.

The peptides and polypeptides of the invention can also be glycosylated. The glycosylation can be added post-translationally either chemically or by cellular biosynthetic mechanisms, wherein the later incorporates the use of known glycosylation motifs, which can be native to the sequence or can be added as a peptide or added in the nucleic acid coding sequence. The glycosylation can be O-linked or N-linked.

The peptides and polypeptides of the invention, as defined above, include all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, in one aspect, a mimetic composition is within the scope of the invention if it has a xylose isomerase activity.

Polypeptide mimetic compositions of the invention can contain any combination of non-natural structural components. In alternative aspect, mimetic compositions of the invention include one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide of the invention can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetraaole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

A polypeptide of the invention can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholinyl-(4-ethyl)carbodiimide or 1-ethyl-3(4-azonia-4,4-dimetholpentyl)carbodiimide. Aspartyl or glutamyl can also be converted to asparaginyl and glutaminyl residues by reaction with ammonium ions. Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl)propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A residue, e.g., an amino acid, of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but also can be referred to as the R— or S— form.

The invention also provides methods for modifying the polypeptides of the invention by either natural processes, such as post-translational processing (e.g., phosphorylation, acylation, etc), or by chemical modification techniques, and the resulting modified polypeptides. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also a given polypeptide may have many types of modifications. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. See, e.g., Creighton, T. E., Proteins—Structure and Molecular Properties 2nd Ed., W.H. Freeman and Company, New York (1993); Posttranslational Covalent Modification of Proteins, B. C. Johnson, Ed., Academic Press, New York, pp. 1-12 (1983).

Solid-phase chemical peptide synthesis methods can also be used to synthesize the polypeptides, or fragments thereof, of the invention. Such method have been known in the art since the early 1960's (Merrifield, R. B., J. Am. Chem. Soc., 85:2149-2154, 1963) (See also Stewart, J. M. and Young, J. D., Solid Phase Peptide Synthesis, 2nd Ed., Pierce Chemical Co., Rockford, Ill., pp. 11-12)) and have recently been employed in commercially available laboratory peptide design and synthesis kits (Cambridge Research Biochemicals). Such commercially available laboratory kits have generally utilized the teachings of H. M. Geysen et al, Proc. Natl. Acad. Sci., USA, 81:3998 (1984) and provide for synthesizing peptides upon the tips of a multitude of “rods” or “pins” all of which are connected to a single plate. When such a system is utilized, a plate of rods or pins is inverted and inserted into a second plate of corresponding wells or reservoirs, which contain solutions for attaching or anchoring an appropriate amino acid to the pin's or rod's tips. By repeating such a process step, i.e., inverting and inserting the rod's and pin's tips into appropriate solutions, amino acids are built into desired peptides. In addition, a number of available FMOC peptide synthesis systems are available. For example, assembly of a polypeptide or fragment can be carried out on a solid support using an Applied Biosystems, Inc. Model 431A™ automated peptide synthesizer. Such equipment provides ready access to the peptides of the invention, either by direct synthesis or by synthesis of a series of fragments that can be coupled using other known techniques.

Exemplary SEQ ID NO:2 has the sequence:

Met Thr Glu Phe Phe Pro Glu Ile Pro Lys Ile Gln
Phe Glu Gly Lys Glu Ser Thr Asn Pro Phe Ala Phe
Lys Phe Tyr Asp Pro Asn Glu Val Ile Asp Gly Lys
Pro Leu Lys Asp His Leu Lys Phe Ser Val Ala Phe
Trp His Thr Phe Val Asn Glu Gly Arg Asp Pro Phe
Gly Asp Pro Thr Ala Asp Arg Pro Trp Asn Lys Tyr
Thr Asp Pro Met Asp Lys Ala Phe Ala Arg Val Asp
Ala Leu Phe Glu Phe Cys Glu Lys Leu Asn Ile Glu
Tyr Phe Cys Phe His Asp Arg Asp Ile Ala Pro Glu
Gly Lys Thr Leu Arg Glu Thr Asn Lys Ile Leu Asp
Lys Val Val Glu Arg Ile Lys Glu Arg Met Lys Asp
Ser Asn Val Lys Leu Leu Trp Gly Thr Ala Asn Leu
Phe Ser His Pro Arg Tyr Met His Gly Ala Ala Thr
Thr Cys Ser Ala Asp Val Phe Ala Tyr Ala Ala Ala
Gln Val Lys Lys Ala Leu Glu Ile Thr Lys Glu Leu
Gly Gly Glu Gly Tyr Val Phe Trp Gly Gly Arg Glu
Gly Tyr Glu Thr Leu Leu Asn Thr Asp Leu Asp Leu
Glu Leu Gly Asn Leu Ala Arg Phe Leu Arg Met Ala
Val Asp Tyr Ala Lys Lys Ile Gly Phe Asn Gly Gln
Phe Leu Ile Glu Pro Lys Pro Lys Glu Pro Thr Lys
His Gln Tyr Asp Phe Asp Val Ala Thr Ala Tyr Ala
Phe Leu Lys Ser His Gly Leu Asp Glu Tyr Phe Lys
Phe Asn Ile Glu Ala Asn His Ala Thr Leu Ala Gly
His Thr Phe Gln His Glu Leu Arg Met Ala Arg Ile
Leu Gly Lys Leu Gly Ser Ile Asp Ala Asn Gln Gly
Asp Leu Leu Leu Gly Trp Asp Thr Asp Gln Phe Pro
Thr Asn Val Tyr Asp Thr Thr Leu Ala Met Tyr Glu
Val Ile Lys Ala Gly Gly Phe Thr Lys Gly Gly Leu
Asn Phe Asp Ala Lys Val Arg Arg Ala Ser Tyr Lys
Val Glu Asp Leu Phe Ile Gly His Ile Ala Gly Met
Asp Thr Phe Ala Leu Gly Phe Lys Ile Ala His Lys
Leu Val Lys Asp Gly Val Phe Asp Lys Phe Ile Glu
Glu Lys Tyr Lys Ser Phe Arg Glu Gly Ile Gly Lys
Glu Ile Val Glu Gly Lys Ala Asp Phe Glu Lys Leu
Glu Ala Tyr Ile Ile Asp Lys Glu Glu Met Glu Leu
Pro Ser Gly Lys Gln Glu Tyr Leu Glu Ser Leu Leu
Asn Ser Tyr Ile Val Lys Thr Ile Ser Glu Leu Arg

Exemplary SEQ ID NO:4 has the sequence:

Met Thr Glu Phe Phe Pro Glu Ile Pro Lys Ile Gln
Phe Glu Gly Lys Glu Ser Asn Asn Pro Leu Ala Phe
Lys Phe Tyr Asp Pro Asp Glu Val Ile Asp Gly Lys
Pro Leu Lys Asp His Leu Lys Phe Ser Val Ala Phe
Trp His Thr Phe Val Asn Glu Gly Arg Asp Pro Phe
Gly Asp Pro Thr Ala Glu Arg Pro Trp Asn Lys Tyr
Ser Asp Pro Met Asp Lys Ala Phe Ala Arg Val Asp
Ala Leu Phe Glu Phe Cys Gln Lys Leu Asn Ile Glu
Tyr Phe Cys Phe His Asp Arg Asp Ile Ala Pro Glu
Gly Lys Thr Leu Arg Glu Thr Asn Lys Ile Leu Asp
Lys Val Val Glu Lys Ile Lys Glu Arg Met Lys Glu
Ser Asn Val Lys Leu Leu Trp Gly Thr Ala Asn Leu
Phe Ser His Pro Arg Tyr Met His Gly Ala Ala Thr
Thr Cys Ser Ala Asp Val Phe Ala Tyr Ala Ala Ala
Gln Val Lys Lys Ala Leu Glu Ile Thr Lys Glu Leu
Gly Gly Glu Gly Tyr Val Phe Trp Gly Gly Arg Glu
Gly Tyr Glu Thr Leu Leu Asn Thr Asp Leu Gly Leu
Glu Leu Glu Asn Leu Ala Arg Phe Leu Arg Met Ala
Val Glu Tyr Ala Lys Lys Ile Gly Phe Asp Gly Gln
Phe Leu Ile Glu Pro Lys Pro Lys Glu Pro Thr Lys
His Gln Tyr Asp Phe Asp Val Ala Thr Ala Tyr Ala
Phe Leu Lys Thr His Asp Leu Asp Glu Tyr Phe Lys
Phe Asn Ile Glu Ala Asn His Ala Thr Leu Ala Gly
His Thr Phe Gln His Glu Leu Arg Met Ala Arg Ile
Leu Gly Lys Phe Gly Ser Ile Asp Ala Asn Gln Gly
Asp Leu Leu Leu Gly Trp Asp Thr Asp Gln Phe Pro
Thr Asn Val Tyr Asp Thr Thr Leu Ala Met Tyr Glu
Val Ile Lys Ala Gly Gly Phe Thr Lys Gly Gly Leu
Asn Phe Asp Ala Lys Val Arg Arg Ala Ser Tyr Lys
Val Glu Asp Leu Phe Ile Gly His Ile Val Gly Ile
Asp Thr Phe Ala Leu Gly Phe Lys Ile Ala Tyr Lys
Leu Val Lys Asp Gly Val Phe Asp Arg Phe Val Glu
Glu Lys Tyr Arg Ser Phe Arg Glu Gly Ile Gly Lys
Glu Ile Leu Glu Gly Lys Ala Asp Phe Glu Lys Leu
Glu Ser Tyr Ile Ile Asp Lys Glu Asp Val Glu Leu
Pro Ser Gly Lys Gln Glu Tyr Leu Glu Ser Leu Leu
Asn Ser Tyr Ile Val Lys Thr Val Ser Glu Leu Arg

Exemplary SEQ ID NO:6 has the sequence:

Met Ala Glu Phe Phe Pro Glu Ile Pro Lys Ile Gln
Phe Glu Gly Lys Glu Ser Thr Asn Pro Phe Ala Phe
Lys Phe Tyr Asp Pro Asn Glu Val Ile Asp Gly Lys
Pro Leu Lys Asp His Leu Lys Phe Ser Val Ala Phe
Trp His Thr Phe Val Asn Glu Gly Arg Asp Pro Phe
Gly Asp Pro Thr Ala Asp Arg Pro Trp Asn Lys Tyr
Thr Asp Pro Met Asp Lys Ala Phe Ala Arg Val Asp
Ala Leu Phe Glu Phe Cys Glu Lys Leu Asn Ile Glu
Tyr Phe Cys Phe His Asp Arg Asp Ile Ala Pro Glu
Gly Lys Thr Leu Arg Glu Thr Asn Lys Ile Leu Asp
Lys Val Val Glu Arg Ile Lys Glu Arg Met Lys Asp
Ser Asn Val Lys Leu Leu Trp Gly Thr Ala Asn Leu
Phe Ser His Pro Arg Tyr Met His Gly Ala Ala Thr
Thr Cys Ser Ala Asp Val Phe Ala Tyr Ala Ala Ala
Gln Val Lys Lys Ala Leu Glu Ile Thr Lys Glu Leu
Gly Gly Glu Gly Tyr Val Phe Trp Gly Gly Arg Glu
Gly Tyr Glu Thr Leu Leu Asn Thr Asp Leu Asp Leu
Glu Leu Gly Asn Leu Ala Arg Phe Leu Arg Met Ala
Val Asp Tyr Ala Lys Lys Ile Gly Phe Asn Gly Gln
Phe Leu Ile Glu Pro Lys Pro Lys Glu Pro Thr Lys
His Gln Tyr Asp Phe Asp Val Ala Thr Ala Tyr Ala
Phe Leu Lys Ser His Gly Leu Asp Glu Tyr Phe Lys
Phe Asn Ile Glu Ala Asn His Ala Thr Leu Ala Gly
His Thr Phe Gln His Glu Leu Arg Met Ala Arg Ile
Leu Gly Lys Leu Gly Ser Ile Asp Ala Asn Gln Gly
Asp Leu Leu Leu Gly Trp Asp Thr Asp Gln Phe Pro
Thr Asn Val Tyr Asp Thr Thr Leu Ala Met Tyr Glu
Val Ile Lys Ala Gly Gly Phe Thr Lys Gly Gly Leu
Asn Phe Asp Ala Lys Val Arg Arg Ala Ser Tyr Lys
Val Glu Asp Leu Phe Ile Gly His Ile Ala Gly Met
Asp Thr Phe Ala Leu Gly Phe Lys Ile Ala His Lys
Leu Val Lys Asp Gly Val Phe Asp Lys Phe Ile Glu
Glu Lys Tyr Lys Ser Phe Arg Glu Gly Ile Gly Lys
Glu Ile Val Glu Gly Lys Ala Asp Phe Glu Lys Leu
Glu Ala Tyr Ile Ile Asp Lys Glu Glu Met Glu Leu
Pro Ser Gly Lys Gln Glu Tyr Leu Glu Ser Leu Leu
Asn Ser Tyr Ile Val Lys Thr Ile Ser Glu Leu Arg

Xylose Isomerases

The invention provides novel xylose isomerases, e.g., proteins comprising SEQ ID NO:2 and SEQ ID NO:4, nucleic acids encoding them, e.g., nucleic acids comprising SEQ ID NO:1 and SEQ ID NO:3, antibodies that bind them, and methods for making and using them. The polypeptides of the invention can have a xylose isomerase activity, e.g., in alternative aspects, an activity of a polypeptide of the invention includes isomerization of xylose to xylulose; isomerization of glucose to fructose; isomerization of a D-glucose to a D-fructose; catalysis of the conversion of D-xylose to an equilibrium mixture of D-xylulose and D-xylose; isomerization of β-D-glucopyranose to β-D-fructopyranose; and/or, isomerization of α-D-glucopyranose to α-D-fructofuranose, or, isomerization of xylulose to xylose; isomerization of fructose to glucose; isomerization of a D-fructose to D-glucose; catalysis of the conversion of an equilibrium mixture of D-xylulose and D-xylose to D-xylose; isomerization of β-D-fructopyranose to β-D-glucopyranose; and/or, isomerization of α-D-fructofuranose to α-D-glucopyranose.

In alternative aspects, the xylose isomerases of the invention can have modified or new activities as compared to the exemplary xylose isomerases or the activities described herein. For example, the invention includes xylose isomerases with and without signal sequences and the signal sequences themselves. The invention includes immobilized xylose isomerases, anti-xylose isomerase antibodies and fragments thereof. The invention provides proteins for inhibiting xylose isomerase activity, e.g., antibodies that bind to the xylose isomerase active site. The invention includes homodimers and heterocomplexes, e.g., fusion proteins, heterodimers, etc., comprising the xylose isomerases of the invention. The invention includes xylose isomerases having activity over a broad range of high and low temperatures and pH's (e.g., acidic and basic aqueous conditions).

In alternative aspects, the xylose isomerase is an isomerase that can catalyze the conversion of xylose to xylulose, glucose to fructose (e.g., D-glucose to D-fructose), β-D-glucopyranose to β-D-fructopyranose or α-D-glucopyranose to α-D-fructofuranose. In one aspect, the enzymes can recognize xylose, glucose, β-D-glucopyranose, α-D-glucopyranose and the like as substrates. However, the enzyme can have a higher K_{cat}/Kₘ for xylose. In order to improve this ratio, in one aspect, site-directed mutagenesis is used to create additional xylose isomerase enzymes with alternative substrate specificity. The can be done, for example, by redesigning the substrate binding region or the active site of the enzyme. In one aspect, xylose isomerases of the invention are more stable at high temperatures, such as 80° C. to 85° C. to 90° C. to 95° C., as compared to xylose isomerases from conventional or moderate organisms. This property is especially important during the production of high-fructose corn syrup because the use of thermostable xylose isomerase in the glucose isomerization process allows the reaction to proceed at higher temperatures. In some aspects, this facilitates the production of syrups with a higher fructose content by shifting the chemical equilibrium towards xylulose, fructose, α-D-fructofuranose, β-D-fructopyranose and the like. In one aspect, the pH optimum for an enzyme of the invention is in the range between pH 4.5 to 5.0 to 5.3 to 5.5 to 6.0 to 6.5. Activity at these relatively acidic conditions makes these exemplary xylose isomerases of the invention useful in the methods of the invention that comprise production of high-fructose corn syrup where liquefaction, saccharification and isomerization steps are combined into a single step. Use of the exemplary xylose isomerases of the invention that are active in acidic conditions can eliminate the need for a pH adjustment for the isomerization step.

Proteins of the present invention can be used within laboratory and industrial settings to catalyze the isomerization of xylose or glucose for a variety of purposes. The proteins can be used alone to provide specific isomerization of fructose to glucose or can be combined with other proteins such as amylases and glucoamylases to provide a “cocktail” for starch hydrolysis with a broad spectrum of activity. Representative laboratory uses include fermentation of xylose and glucose by genetically engineered bacteria containing xylose isomerase. Within industry, the proteins of the present invention can be used within the large-scale preparation of high-fructose syrups (see industrial applications below).

Various proteins of the invention have a xylose isomerase activity under various conditions. The invention provides methods of making xylose isomerases with different catalytic efficiency and stabilities towards temperature, oxidizing agents and pH conditions. These methods can use, e.g., the techniques of site-directed mutagenesis and/or random mutagenesis. In one aspect, directed evolution can be used to produce xylose isomerases with alternative specificities and stability.

The proteins of the invention are used in methods of the invention that can identify xylose isomerase modulators, e.g., activators or inhibitors. Briefly, test samples (e.g., compounds, such as members of peptide or combinatorial libraries, broths, extracts, and the like) are added to xylose isomerase assays to determine their ability to modulate, e.g., inhibit or activate, substrate cleavage. These inhibitors can be used in industry and research to reduce or prevent undesired isomerization. Modulators found using the methods of the invention can be used to alter (e.g., decrease or increase) the spectrum of activity of a xylose isomerase.

The invention also provides methods of discovering xylose isomerases using the nucleic acids, polypeptides and antibodies of the invention. In one aspect, lambda phage libraries are screened for expression-based discovery of xylose isomerases. In one aspect, the invention uses lambda phage libraries in screening to allow detection of toxic clones; improved access to substrate; reduced need for engineering a host, by-passing the potential for any bias resulting from mass excision of the library; and, faster growth at low clone densities. Screening of lambda phage libraries can be in liquid phase or in solid phase. In one aspect, the invention provides screening in liquid phase. This gives a greater flexibility in assay conditions; additional substrate flexibility; higher sensitivity for weak clones; and ease of automation over solid phase screening.

The invention provides screening methods using the proteins and nucleic acids of the invention involving robotic automation. This enables the execution of many thousands of biocatalytic reactions and screening assays in a short period of time, e.g., per day, as well as ensuring a high level of accuracy and reproducibility (see discussion of arrays, below). As a result, a library of derivative compounds can be produced in a matter of weeks.

The invention includes xylose isomerase enzymes which are non-naturally occurring xylose isomerases having a different xylose isomerase activity, stability, substrate specificity, pH profile and/or performance characteristic as compared to the non-naturally occurring xylose isomerase. These xylose isomerases have an amino acid sequence not found in nature. They can be derived by substitution of a plurality of amino acid residues of a precursor xylose isomerase with different amino acids. The precursor xylose isomerase may be a naturally-occurring xylose isomerase or a recombinant xylose isomerase. In one aspect, the xylose isomerase variants encompass the substitution of any of the naturally occurring L-amino acids at the designated amino acid residue positions.

Hybrid Xylose Isomerases and Peptide Libraries

In one aspect, the invention provides hybrid xylose isomerases and fusion proteins, including peptide libraries, comprising sequences of the invention. The peptide libraries of the invention can be used to isolate peptide modulators (e.g., activators or inhibitors) of targets, such as xylose isomerase substrates, receptors, enzymes. The peptide libraries of the invention can be used to identify formal binding partners of targets, such as ligands, e.g., cytokines, hormones and the like.

In one aspect, the fusion proteins of the invention (e.g., the peptide moiety) are conformationally stabilized (relative to linear peptides) to allow a higher binding affinity for targets. The invention provides fusions of xylose isomerases of the invention and other peptides, including known and random peptides. They can be fused in such a manner that the structure of the xylose isomerases are not significantly perturbed and the peptide is metabolically or structurally conformationally stabilized. This allows the creation of a peptide library that is easily monitored both for its presence within cells and its quantity.

Amino acid sequence variants of the invention can be characterized by a predetermined nature of the variation, a feature that sets them apart from a naturally occurring form, e.g., an allelic or interspecies variation of a xylose isomerase sequence. In one aspect, the variants of the invention exhibit the same qualitative biological activity as the naturally occurring analogue. Alternatively, the variants can be selected for having modified characteristics. In one aspect, while the site or region for introducing an amino acid sequence variation is predetermined, the mutation per se need not be predetermined. For example, in order to optimize the performance of a mutation at a given site, random mutagenesis may be conducted at the target codon or region and the expressed xylose isomerase variants screened for the optimal combination of desired activity. Techniques for making substitution mutations at predetermined sites in DNA having a known sequence are well known, as discussed herein for example, M13 primer mutagenesis and PCR mutagenesis. Screening of the mutants can be done using assays of proteolytic activities. In alternative aspects, amino acid substitutions can be single residues; insertions can be on the order of from about 1 to 20 amino acids, although considerably larger insertions can be done. Deletions can range from about 1 to about 20, 30, 40, 50, 60, 70 residues or more. To obtain a final derivative with the optimal properties, substitutions, deletions, insertions or any combination thereof may be used. Generally, these changes are done on a few amino acids to minimize the alteration of the molecule. However, larger changes may be tolerated in certain circumstances.

The invention provides xylose isomerases where the structure of the polypeptide backbone, the secondary or the tertiary structure, e.g., an alpha-helical or beta-sheet structure, has been modified. In one aspect, the charge or hydrophobicity has been modified. In one aspect, the bulk of a side chain has been modified. Substantial changes in function or immunological identity are made by selecting substitutions that are less conservative. For example, substitutions can be made which more significantly affect: the structure of the polypeptide backbone in the area of the alteration, for example a alpha-helical or a beta-sheet structure; a charge or a hydrophobic site of the molecule, which can be at an active site; or a side chain. The invention provides substitutions in polypeptide of the invention where (a) a hydrophilic residues, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine. The variants can exhibit the same qualitative biological activity (i.e. xylose isomerase activity) although variants can be selected to modify the characteristics of the xylose isomerases as needed.

In one aspect, xylose isomerases of the invention comprise epitopes or purification tags, signal sequences or other fusion sequences, etc. In one aspect, the xylose isomerases of the invention can be fused to a random peptide to form a fusion polypeptide. By “fused” or “operably linked” herein is meant that the random peptide and the xylose isomerase are linked together, in such a manner as to minimize the disruption to the stability of the xylose isomerase structure, e.g., it retains xylose isomerase activity. The fusion polypeptide (or fusion polynucleotide encoding the fusion polypeptide) can comprise further components as well, including multiple peptides at multiple loops.

In one aspect, the peptides (e.g., xylose isomerase subsequences) and nucleic acids encoding them are randomized, either fully randomized or they are biased in their randomization, e.g. in nucleotide/residue frequency generally or per position. “Randomized” means that each nucleic acid and peptide consists of essentially random nucleotides and amino acids, respectively. In one aspect, the nucleic acids which give rise to the peptides can be chemically synthesized, and thus may incorporate any nucleotide at any position. Thus, when the nucleic acids are expressed to form peptides, any amino acid residue may be incorporated at any position. The synthetic process can be designed to generate randomized nucleic acids, to allow the formation of all or most of the possible combinations over the length of the nucleic acid, thus forming a library of randomized nucleic acids. The library can provide a sufficiently structurally diverse population of randomized expression products to affect a probabilistically sufficient range of cellular responses to provide one or more cells exhibiting a desired response. Thus, the invention provides an interaction library large enough so that at least one of its members will have a structure that gives it affinity for some molecule, protein, or other factor.

Screening Methodologies and “On-Line” Monitoring Devices

In practicing the methods of the invention, a variety of apparatus and methodologies can be used to in conjunction with the polypeptides and nucleic acids of the invention, e.g., to screen polypeptides for xylose isomerase activity, to screen compounds as potential activators or inhibitors of a xylose isomerase activity, for antibodies that bind to a polypeptide of the invention, for nucleic acids that hybridize to a nucleic acid of the invention, to screen for cells expressing a polypeptide of the invention and the like.

Capillary Arrays

Capillary arrays, such as the GIGAMATRIX™, Diversa Corporation, San Diego, Calif., can be used to in the methods of the invention. Nucleic acids or polypeptides of the invention can be immobilized to or applied to an array, including capillary arrays. Arrays can be used to screen for or monitor libraries of compositions (e.g., small molecules, antibodies, nucleic acids, etc.) for their ability to bind to or modulate the activity of a nucleic acid or a polypeptide of the invention. Capillary arrays provide another system for holding and screening samples. For example, a sample screening apparatus can include a plurality of capillaries formed into an array of adjacent capillaries, wherein each capillary comprises at least one wall defining a lumen for retaining a sample. The apparatus can further include interstitial material disposed between adjacent capillaries in the array, and one or more reference indicia formed within of the interstitial material. A capillary for screening a sample, wherein the capillary is adapted for being bound in an array of capillaries, can include a first wall defining a lumen for retaining the sample, and a second wall formed of a filtering material, for filtering excitation energy provided to the lumen to excite the sample.

A polypeptide or nucleic acid, e.g., a ligand, can be introduced into a first component into at least a portion of a capillary of a capillary array. Each capillary of the capillary array can comprise at least one wall defining a lumen for retaining the first component. An air bubble can be introduced into the capillary behind the first component. A second component can be introduced into the capillary, wherein the second component is separated from the first component by the air bubble. A sample of interest can be introduced as a first liquid labeled with a detectable particle into a capillary of a capillary array, wherein each capillary of the capillary array comprises at least one wall defining a lumen for retaining the first liquid and the detectable particle, and wherein the at least one wall is coated with a binding material for binding the detectable particle to the at least one wall. The method can further include removing the first liquid from the capillary tube, wherein the bound detectable particle is maintained within the capillary, and introducing a second liquid into the capillary tube.

The capillary array can include a plurality of individual capillaries comprising at least one outer wall defining a lumen. The outer wall of the capillary can be one or more walls fused together. Similarly, the wall can define a lumen that is cylindrical, square, hexagonal or any other geometric shape so long as the walls form a lumen for retention of a liquid or sample. The capillaries of the capillary array can be held together in close proximity to form a planar structure. The capillaries can be bound together, by being fused (e.g., where the capillaries are made of glass), glued, bonded, or clamped side-by-side. The capillary array can be formed of any number of individual capillaries, for example, a range from 100 to 4,000,000 capillaries. A capillary array can form a micro titer plate having about 100,000 or more individual capillaries bound together.

Arrays, or “Biochips”

Nucleic acids or polypeptides of the invention can be immobilized to or applied to an array. Arrays can be used to screen for or monitor libraries of compositions (e.g., small molecules, antibodies, nucleic acids, etc.) for their ability to bind to or modulate the activity of a nucleic acid or a polypeptide of the invention. For example, in one aspect of the invention, a monitored parameter is transcript expression of a xylose isomerase gene. One or more, or, all the transcripts of a cell can be measured by hybridization of a sample comprising transcripts of the cell, or, nucleic acids representative of or complementary to transcripts of a cell, by hybridization to immobilized nucleic acids on an array, or “biochip.” By using an “array” of nucleic acids on a microchip, some or all of the transcripts of a cell can be simultaneously quantified. Alternatively, arrays comprising genomic nucleic acid can also be used to determine the genotype of a newly engineered strain made by the methods of the invention. Polypeptide arrays” can also be used to simultaneously quantify a plurality of proteins. The present invention can be practiced with any known “array,” also referred to as a “microarray” or “nucleic acid array” or “polypeptide array” or “antibody array” or “biochip,” or variation thereof. Arrays are generically a plurality of “spots” or “target elements,” each target element comprising a defined amount of one or more biological molecules, e.g., oligonucleotides, immobilized onto a defined area of a substrate surface for specific binding to a sample molecule, e.g., mRNA transcripts.

In one aspect, the xylose isomerases are used as immobilized forms. Any immobilization method can be used, e.g., immobilization upon an inert support such as diethylaminoethyl-cellulose, porous glass, chitin or cells. Cells that express glucose isomerase of the invention can be immobilized by cross-linking, e.g. with glutaraldehyde to a substrate surface.

In practicing the methods of the invention, any known array and/or method of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32. See also published U.S. patent applications Nos. 20010018642; 20010019827; 20010016322; 20010014449; 20010014448; 20010012537; 20010008765.

Antibodies and Antibody-Based Screening Methods

The invention provides isolated or recombinant antibodies that specifically bind to a xylose isomerase of the invention. These antibodies can be used to isolate, identify or quantify the xylose isomerase of the invention or related polypeptides. These antibodies can be used to isolate other polypeptides within the scope the invention or other related xylose isomerases.

The antibodies can be used in immunoprecipitation, staining, immunoaffinity columns, and the like. If desired, nucleic acid sequences encoding for specific antigens can be generated by immunization followed by isolation of polypeptide or nucleic acid, amplification or cloning and immobilization of polypeptide onto an array of the invention. Alternatively, the methods of the invention can be used to modify the structure of an antibody produced by a cell to be modified, e.g., an antibody's affinity can be increased or decreased. Furthermore, the ability to make or modify antibodies can be a phenotype engineered into a cell by the methods of the invention.

Methods of immunization, producing and isolating antibodies (polyclonal and monoclonal) are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

Polypeptides or peptides can be used to generate antibodies, which bind specifically to the polypeptides of the invention. The resulting antibodies may be used in immunoaffinity chromatography procedures to isolate or purify the polypeptide or to determine whether the polypeptide is present in a biological sample. In such procedures, a protein preparation, such as an extract, or a biological sample is contacted with an antibody capable of specifically binding to one of the polypeptides of the invention.

In immunoaffinity procedures, the antibody is attached to a solid support, such as a bead or other column matrix. The protein preparation is placed in contact with the antibody under conditions in which the antibody specifically binds to one of the polypeptides of the invention. After a wash to remove non-specifically bound proteins, the specifically bound polypeptides are eluted.

The ability of proteins in a biological sample to bind to the antibody may be determined using any of a variety of procedures familiar to those skilled in the art. For example, binding may be determined by labeling the antibody with a detectable label such as a fluorescent agent, an enzymatic label, or a radioisotope. Alternatively, binding of the antibody to the sample may be detected using a secondary antibody having such a detectable label thereon. Particular assays include ELISA assays, sandwich assays, radioimmunoassays, and Western Blots.

Polyclonal antibodies generated against the polypeptides of the invention can be obtained by direct injection of the polypeptides into an animal or by administering the polypeptides to a non-human animal. The antibody so obtained will then bind the polypeptide itself. In this manner, even a sequence encoding only a fragment of the polypeptide can be used to generate antibodies which may bind to the whole native polypeptide. Such antibodies can then be used to isolate the polypeptide from cells expressing that polypeptide.

For preparation of monoclonal antibodies, any technique which provides antibodies produced by continuous cell line cultures can be used. Examples include the hybridoma technique, the trioma technique, the human B-cell hybridoma technique, and the EBV-hybridoma technique (see, e.g., Cole (1985) in Monoclonal Antibodies and Cancer Therapy, Alan R. Liss, Inc., pp. 77-96).

Techniques described for the production of single chain antibodies (see, e.g., U.S. Pat. No. 4,946,778) can be adapted to produce single chain antibodies to the polypeptides of the invention. Alternatively, transgenic mice may be used to express humanized antibodies to these polypeptides or fragments thereof.

Antibodies generated against the polypeptides of the invention (including anti-idiotype antibodies) may be used in screening for similar polypeptides from other organisms and samples. In such techniques, polypeptides from the organism are contacted with the antibody and those polypeptides which specifically bind the antibody are detected. Any of the procedures described above may be used to detect antibody binding.

Kits

The invention provides kits comprising the compositions, e.g., nucleic acids, expression cassettes, vectors, cells, polypeptides (e.g., xylose isomerases) and/or antibodies of the invention. The kits also can contain instructional material teaching the methodologies and industrial uses of the invention, as described herein.

Measuring Metabolic Parameters

The methods of the invention provide whole cell evolution, or whole cell engineering, of a cell to develop a new cell strain having a new phenotype by modifying the genetic composition of the cell, where the genetic composition is modified by addition to the cell of a nucleic acid. To detect the new phenotype, at least one metabolic parameter of a modified cell is monitored in the cell in a “real time” or “on-line” time frame. In one aspect, a plurality of cells, such as a cell culture, is monitored in “real time” or “on-line.” In one aspect, a plurality of metabolic parameters is monitored in “real time” or “on-line.”Metabolic parameters can be monitored using the fluorescent polypeptides of the invention.

Metabolic flux analysis (MFA) is based on a known biochemistry framework. A linearly independent metabolic matrix is constructed based on the law of mass conservation and on the pseudo-steady state hypothesis (PSSH) on the intracellular metabolites. In practicing the methods of the invention, metabolic networks are established, including the:

identity of all pathway substrates, products and intermediary metabolites

identity of all the chemical reactions interconverting the pathway metabolites, the stoichiometry of the pathway reactions,

identity of all the enzymes catalyzing the reactions, the enzyme reaction kinetics,

the regulatory interactions between pathway components, e.g. allosteric interactions, enzyme-enzyme interactions etc,

intracellular compartmentalization of enzymes or any other supramolecular organization of the enzymes, and,

the presence of any concentration gradients of metabolites, enzymes or effector molecules or diffusion barriers to their movement.

Once the metabolic network for a given strain is built, mathematic presentation by matrix notion can be introduced to estimate the intracellular metabolic fluxes if the on-line metabolome data is available. Metabolic phenotype relies on the changes of the whole metabolic network within a cell. Metabolic phenotype relies on the change of pathway utilization with respect to environmental conditions, genetic regulation, developmental state and the genotype, etc. In one aspect of the methods of the invention, after the on-line MFA calculation, the dynamic behavior of the cells, their phenotype and other properties are analyzed by investigating the pathway utilization. For example, if the glucose supply is increased and the oxygen decreased during the yeast fermentation, the utilization of respiratory pathways will be reduced and/or stopped, and the utilization of the fermentative pathways will dominate. Control of physiological state of cell cultures will become possible after the pathway analysis. The methods of the invention can help determine how to manipulate the fermentation by determining how to change the substrate supply, temperature, use of inducers, etc. to control the physiological state of cells to move along desirable direction. In practicing the methods of the invention, the MFA results can also be compared with transcriptome and proteome data to design experiments and protocols for metabolic engineering or gene shuffling, etc.

In practicing the methods of the invention, any modified or new phenotype can be conferred and detected, including new or improved characteristics in the cell. Any aspect of metabolism or growth can be monitored.

Monitoring Expression of an mRNA Transcript

In one aspect of the invention, the engineered phenotype comprises increasing or decreasing the expression of an mRNA transcript or generating new transcripts in a cell. This increased or decreased expression can be traced by use of a xylose isomerase of the invention. mRNA transcripts, or messages, also can be detected and quantified by any method known in the art, including, e.g., Northern blots, quantitative amplification reactions, hybridization to arrays, and the like. Quantitative amplification reactions include, e.g., quantitative PCR, including, e.g., quantitative reverse transcription polymerase chain reaction, or RT-PCR; quantitative real time RT-PCR, or “real-time kinetic RT-PCR” (see, e.g., Kreuzer (2001) Br. J. Haematol. 114:313-318; Xia (2001) Transplantation 72:907-914).

In one aspect of the invention, the engineered phenotype is generated by knocking out expression of a homologous gene. The gene's coding sequence or one or more transcriptional control elements can be knocked out, e.g., promoters or enhancers. Thus, the expression of a transcript can be completely ablated or only decreased.

In one aspect of the invention, the engineered phenotype comprises increasing the expression of a homologous gene. This can be effected by knocking out of a negative control element, including a transcriptional regulatory element acting in cis- or trans-, or, mutagenizing a positive control element. One or more, or, all the transcripts of a cell can be measured by hybridization of a sample comprising transcripts of the cell, or, nucleic acids representative of or complementary to transcripts of a cell, by hybridization to immobilized nucleic acids on an array.

Monitoring Expression of a Polypeptides, Peptides and Amino Acids

In one aspect of the invention, the engineered phenotype comprises increasing or decreasing the expression of a polypeptide or generating new polypeptides in a cell. This increased or decreased expression can be traced by use of a xylose isomerase of the invention. Polypeptides, peptides and amino acids also can be detected and quantified by any method known in the art, including, e.g., nuclear magnetic resonance (NMR), spectrophotometry, radiography (protein radiolabeling), electrophoresis, capillary electrophoresis, high performance liquid chromatography (HPLC), thin layer chromatography (TLC), hyperdiffusion chromatography, various immunological methods, e.g. immunoprecipitation, immunodiffusion, immuno-electrophoresis, radioimmunoassays (RIAs), enzyme-linked immunosorbent assays (ELISAs), immuno-fluorescent assays, gel electrophoresis (e.g., SDS-PAGE), staining with antibodies, fluorescent activated cell sorter (FACS), pyrolysis mass spectrometry, Fourier-Transform Infrared Spectrometry, Raman spectrometry, GC-MS, and LC-Electrospray and cap-LC-tandem-electrospray mass spectrometries, and the like. Novel bioactivities can also be screened using methods, or variations thereof, described in U.S. Pat. No. 6,057,103. Furthermore, as discussed below in detail, one or more, or, all the polypeptides of a cell can be measured using a protein array.

### INDUSTRIAL APPLICATIONS

High-Fructose Syrups

In alternative aspects, the invention provides processes of converting glucose to fructose, such as D-fructose, xylose to xylulose, α-D-glucopyranose to α-D-fructofuranose and β-D-glucopyranose to β-D-fructopyranose. Thus, the invention provides methods for making compositions comprising these “sweet” sugars, e.g., syrups, such as high fructose syrups, e.g., high fructose corn syrup (HFCS). Fructose and related compounds are very sweet natural sugars. Syrups produced by these processes can be used in place of sucrose (cane sugar) in many food applications.

The invention provides methods comprising processing starch to fructose. In one aspect, the methods of the invention comprise four steps: liquefaction of granular starch, saccharification of the liquefied starch into dextrose, purification, and isomerization to fructose. In one aspect, the processing methods of the invention, e.g., the processing of starch to glucose and HFCS, makes use of a xylose isomerase of the invention and amylases, such as glucoamylases. Each enzymes can be designed or chosen to have its own pH and temperature optimum. In one aspect the first step, known as liquefaction, the incoming starch slurry is adjusted to pH 6 with NaOH and Ca²⁺ is added for enzyme stability. Alpha-amylase is added and the starch is heated by jet cooker and held at 95-105° C. for up to three hours. An amylase can hydrolyse α-1,4 linkages of starch to maltodextrins with an average chain length of about 8 to 12 glucose units. In one aspect the second step, saccharification, the pH is adjusted down to 4.5 with HCl and cooled to 60° C. Glucoamylase then removes single glucose units from the maltodextrins until it is completely hydrolyzed to glucose. This step can take between about 24 to 96 hours. In one aspect the third step, isomerization, NaOH is used to bring the pH to above 7 and Mg²⁺ is added. The glucose syrup is then passed over an immobilized xylose isomerase of the invention, which isomerizes the keto-sugar (glucose) to the aldo-sugar (fructose). The result can be a product stream consisting of about 42% fructose.

In one aspect, the invention provides methods for treating food grade glucose, e.g., enzymatic hydrolysates of corn starch, i.e., corn syrup of commerce, using the enzymes of the invention. D-glucose is generally considered to be 60 to 80% as sweet as sucrose, on a weight basis, and is comparatively insoluble. Batches of 97DE glucose syrup at the final commercial concentration (71% w/w) must be kept warm to prevent crystallization or diluted to concentrations that are microbiologically insecure. Fructose is 30% sweeter than sucrose, on a weight basis, and twice as soluble as glucose at low temperatures, so a 50% conversion of glucose to fructose.

In one aspect, xylose isomerases of the invention are used in immobilized forms. The xylose isomerases of the invention can be immobilized on any support or substrate surface, e.g., an inert support, such as diethylaminoethyl-cellulose, porous glass or chitin (see discussion on arrays, above). Alternatively, xylose isomerases of the invention can be immobilized by cross-linking, e.g. with glutaraldehyde to, e.g., a cell.

The invention incorporates all protocols for the enzymatic conversion of glucose to fructose, xylose to xylulose, α-D-glucopyranose to α-D-fructofuranose and β-D-glucopyranose to β-D-fructopyranose, and the like, e.g., those discussed in Hamilton, et al. “Glucose Isomerase a Case Study of Enzyme-Catalyzed Process Technology”, Immobilized Enzymes in Food and Microbial Processes, Olson et al., Plenum Press, N.Y., (1974), pp. 94-106, 112, 115-137; Antrim, et al., “Glucose Isomerase Production of High-Fructose Syrups”, Applied Biochemistry and Bioengineering, Vol. 2, Academic Press (1979); Chen, et al., “Glucose Isomerase (a Review)”, Process Biochem., (1980), pp. 30-35; Chen, et al. “Glucose Isomerase (a Review)”, Process Biochem., (1980), pp. 36-41; Nordahl, et al., “Fructose Manufacture from Glucose by Immobilized Glucose Isomerase”, Chem. Abstracts, Vol. 82, (1975), Abs. No. 110316h; and Takasaki, “Fructose Production Glucose Isomerase”, Chem. Abstracts, Vol. 82, (1975), Abs. No. 110316h; and Takasaki, “Fructose Production by Glucose Isomerase”, Chem. Abstracts, Vol. 81, (1974), Abs. No. 76474a; U.S. Pat. Nos. 3,616,221; 3,694,314; 3,708,397; 3,715,276; 3,788,945; 3,826,714; 3,843,442; 3,909,354; 3,960,663; 4,144,127; 4,308,349; 5,219,751; 5,656,497; and 6,372,476.

The invention provides xylose isomerases (glucose isomerases) that have activity at temperatures of between about 80° C. to 140° C. and processes for making fructose using these enzymes at these elevated temperatures. The levels of fructose achievable by the isomerization of glucose with xylose isomerase can be limited by the equilibrium of the isomerization reaction. At 65° C., the equilibrium of the reaction can be about 51% fructose by weight from a starting substrate of pure dextrose. The conversion of glucose to fructose can be done at 60° C. to 75° C. and at a pH between 7 and 9. In this case, about 42% yield of fructose is obtained because of the equilibrium between glucose and fructose. One way to shift this equilibrium towards fructose is to increase the temperature of the isomerization reaction. At higher temperatures the equilibrium becomes more favorable. Thus, the invention provides an enzymatic xylose isomerase (glucose isomerase) process at temperatures of between about 80° C. to 120° C. or about 90° C. to 140° C., or, any variation in between. This method of the invention can be used to directly provide high fructose syrups, e.g., high fructose corn syrups (HFCS). These syrups can contain about 53 to 60 weight percent fructose on a dry basis. This can eliminate the need for fractionation and recycling.

In one aspect, the invention provides xylose isomerases (glucose isomerases) that have activity at temperatures of between about 80° C. to 140° C. and at low pH (e.g., acidic aqueous conditions) and processes for making fructose using these enzymes at these elevated temperatures. In this aspect of the methods of the invention (processes at high temperatures and acidic conditions), low levels or no by-products such as psicose, colored products, color precursors, fructose dianhydrides, mannose, tagatose, and acids are formed. Therefore, enzymes of the present invention provide a great advantage since these xylose isomerases can be used at higher temperatures and at generally lower pH, thereby allowing obtaining fructose syrups with higher fructose content.

Food Industry

The enzymes of the invention have numerous applications in the food processing industry. The invention provides foods comprising a polypeptide of the invention and methods for making and processing foods using the enzymes of the invention. For example, high conversion syrups improve moisture retention and color control in a final product. In one aspect, xylose isomerases of the invention are used to obtain high fructose syrups, which in turn are used in various foods, e.g., frosting and fillings, for moisture retention and color control. The invention provides beverages (e.g., soft drinks, alcoholic beverages) comprising high fructose syrups made by and processes using enzymes of the invention. Xylose isomerases of the present invention can be used in production of alcohol and alcoholic beverages. Fructose syrups made by the methods of the invention can be used as fermentation boosters in alcohol fermentation processes.

The invention provides ice cream comprising high fructose syrups made by and processes using enzymes of the invention. High fructose syrups made by processes of the invention are used as crystal and texture controllers and softness and freezing controllers. High fructose syrups made by processes of the invention are used to improve the texture and palatability of foods, e.g., ice cream, to enhance flavors. High fructose syrups made by processes of the invention are used to depress freezing points of foods, e.g., ice creams. High fructose syrups made by processes of the invention are used as sucrose replacements. High fructose syrups made by processes of the invention are used in confectioneries, e.g., candies, jellied fruit products. High fructose syrups made by processes of the invention are used as preserving agents, additives to contribute flavor and additives for gelling. High fructose syrups made by processes of the invention are used as sweeteners and agents to increase osmotic pressure of foods and to increase shelf life of foods.

The invention also provides transgenic plants and seeds comprising a nucleic acid of the invention wherein a recombinant enzyme of the invention is expressed. In one aspect, enzymes of the invention are expressed in starch granules, e.g., of grain such as corn, wheat or potato, such that the enzymes will be co-purified with the starch, e.g. in a standard wet milling operation. Subsequently, when the grain is cooked, for example, when potato is heated and mashed, starch will be hydrolyzed to glucose, which, in turn, will be isomerized into fructose to give the food a sweeter flavor. In one aspect, the xylose isomerases expressed in the transgenic plants and seeds are thermostable or are activated only when heated.

### OTHER INDUSTRIAL APPLICATIONS

The enzymes of the present invention can be used in preparation of insecticides, as discussed, for example, in U.S. Pat. No. 6,162,825. Without regard for the toxicant in the instant bait composition, it has been found that bait compositions having ultra high fructose to glucose ratios are more efficient than those with lower fructose to glucose ratios. For example, the invention provides a cockroach bait containing fructose to glucose ratios in excess of about 9:1, respectively.

The xylose isomerases of the invention can be used to convert glucose to fructose using any method, e.g., as described by Hamilton, et al. “Glucose Isomerase a Case Study of Enzyme-Catalyzed Process Technology”, Immobilized Enzymes in Food and Microbial Processes, Olson et al., Plenum Press, N.Y., (1974), pp. 94-106, 112, 115-137; Antrim, et al., “Glucose Isomerase Production of High-Fructose Syrups”, Applied Biochemistry and Bioengineering, Vol. 2, Academic Press (1979); Chen, et al., “Glucose Isomerase (a Review)”, Process Biochem., (1980), pp. 30-35; Chen, et al. “Glucose Isomerase (a Review)”, Process Biochem., (1980), pp. 36-41; Nordahl, et al., “Fructose Manufacture from Glucose by Immobilized Glucose Isomerase”, Chem. Abstracts, Vol. 82, (1975), Abs. No. 110316h; and Takasaki, “Fructose Production Glucose Isomerase”, Chem. Abstracts, Vol. 82, (1975), Abs. No. 110316h; and Takasaki, “Fructose Production by Glucose Isomerase”, Chem. Abstracts, Vol. 81, (1974), Abs. No. 76474a; U.S. Pat. Nos. 3,616,221; 3,694,314; 3,708,397; 3,715,276; 3,788,945; 3,826,714; 3,843,442; 3,909,354; 3,960,663; 4,144,127; 4,308,349; 5,219,751; 5,656,497; 6,372,476.

The invention will be further described with reference to the following examples; however, it is to be understood that the invention is not limited to such examples.

### EXAMPLES

### Example 1

### An Exemplary Starch Processing Industrial Protocol

The following example describes an exemplary starch processing industrial protocol using a xylose isomerase (i.e., glucose isomerase) of the invention.

This exemplary starch processing industrial protocol of the invention incorporates the xylose isomerase processing of starch to glucose. It makes use of three enzymes: xylose isomerase, glucoamylase, and a glucose isomerase of the invention. Each of these enzymes has its own pH and temperature optimum which requires that the operation be broken up into three enzymatic steps. In the first step, known as liquefaction, the incoming starch slurry is adjusted to pH 6 with NaOH and Ca2+ is added for enzyme stability. An alpha-amylase (e.g., from Bacillus lichenoformis) is added and the starch is heated by jet cooker and held at 95-105° C. for up to three hours. The xylose isomerase hydrolyses α-1,4 linkages of starch to maltodextrins with an average chain length of 8-12 glucose units. In the second step, saccharification, the pH is adjusted down to 4.5 with HCl and cooled to 60° C. Glucoamylase (e.g., from Aspergillus niger) then removes single glucose units from the maltodextrins until it is completely hydrolyzed to glucose. This step takes about 24 to 96 hours. In the third step, isomerization, NaOH is used to bring the pH to above 7 and Mg²⁺ is added. The glucose syrup is then passed over immobilized a xylose isomerase of the invention which isomerizes the keto-sugar (glucose) to the aldo-sugar (fructose). The result is a product stream consisting of about 42% fructose.

The levels of fructose achievable by the isomerization of glucose with glucose isomerase can be limited by the equilibrium of the isomerization reaction. At 65° C., the equilibrium of the reaction can be approximately 51% fructose by weight from a starting substrate of pure dextrose. Under standard conditions, the conversion of glucose to fructose is done at 60° C. to 75° C. and at a pH between 7 and 9. In this exemplary protocol, normally only 42% of fructose is obtained because of the equilibrium between glucose and fructose. To shift this equilibrium towards fructose, the temperature is increased.

To attain syrups of higher fructose content, fractionation systems can be employed. At higher temperatures, however, the equilibrium becomes more favorable. For example, an enzymatic glucose isomerase process capable of being operated at temperatures of from about 90° C. to 140° C. can be used to directly provide high fructose corn syrups containing 53-60 weight percent fructose on a dry basis to eliminate the need for fractionation and recycling.

### Example 2

### An Exemplary Method to Test for Xylose Isomerase Activity

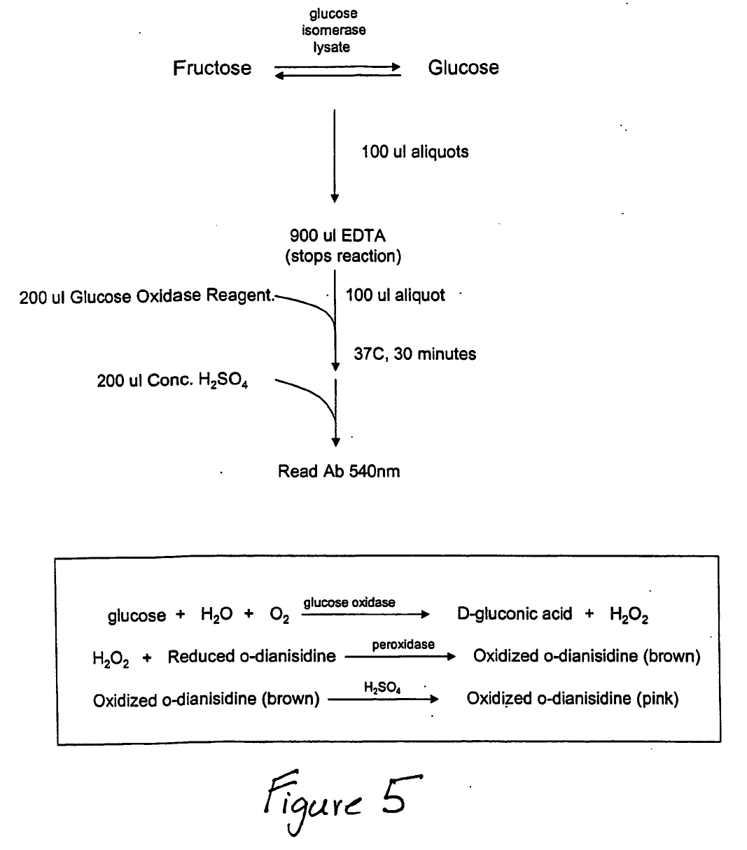

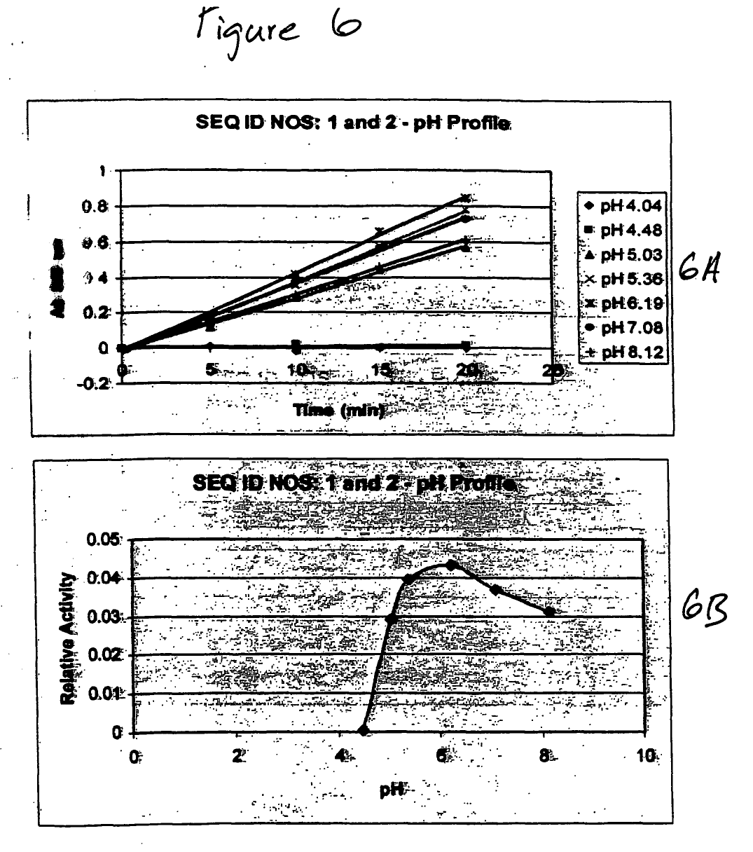

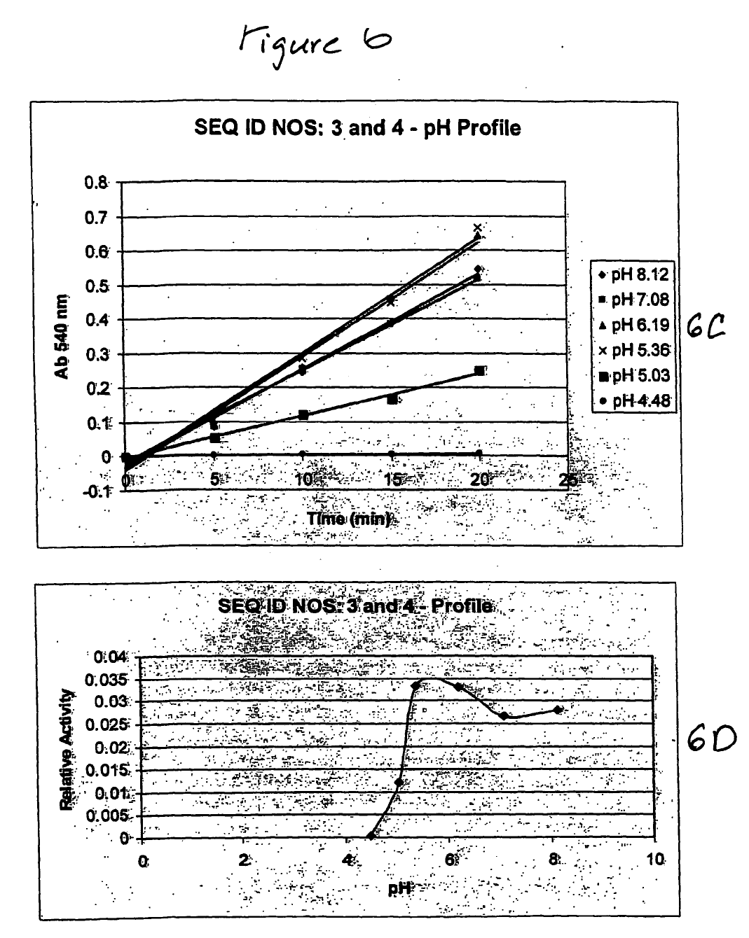

The following example describes an exemplary method to test for xylose isomerase activity to determine if a polypeptide is within the scope of the invention, as illustrated in the schematic diagram of FIG. 5.

As noted in FIG. 5, a xylose isomerase (i.e., glucose isomerase) lysate from a host cell or in vitro reaction recombinantly expressing the enzyme is incubated with glucose, fructose or a combination thereof. The lysate is then incubated under various conditions. Aliquots (e.g., 100 μl aliquots) of the lysate are taken and the reaction is stopped with EDTA. Alternatively, the reaction can be stopped before quenching with EDTA. Glucose oxidase reagent is added (e.g., 200 μl per 100 μl aliquot). As noted in FIG. 5, the reaction catalyzed by glucose oxidase is glucose+water+O₂→D-gluconic acid and hydrogen peroxide. The samples are incubated, e.g., at 37° C. for 30 minutes. Addition of peroxidase catalyzes the reaction H₂O₂+reduced o-dianisidine→oxidized o-dianisidine (brown). Concentrated H₂SO₄ is added (e.g., 200 μl per sample). As noted in FIG. 5, the H₂SO₄-mediated reaction is oxidized o-dianisidine (brown)→oxidized o-dianisidine (yellow). The samples are then read at Ab 540 nm.

### Example 3

### Activity Testing of Exemplary Xylose Isomerases of the Invention

The following example describes activity testing of exemplary xylose isomerases of the invention, as illustrated in the schematic diagrams of FIGS. 6 through 9.

For a series of tests profiling the activities of the exemplary proteins having a sequence as set forth in SEQ ID NO:2 and SEQ ID NO:4 under different pH conditions, reactions were performed in either phosphate buffer at pH 6.19, 7.08 or 8.12 or acetate buffer at pH 4.04, 4.48, 5.03 or 5.36. A 20 μl aliquot of resuspended enzyme (having a sequence as set forth in SEQ ID NO:2 or SEQ ID NO:4) was added to a 500 μl reaction buffer (25 mM buffer, 10 mM fructose, 0.5 mM CoCl₂, 0.5 mM MgCl₂) at 80° C. 100 μl aliquots were removed to 900 μl mM EDTA on ice at five minute time points. Glucose levels of a 100 μl aliquot were determined from each time point using Sigma's glucose assay kit (Sigma-Aldrich, St. Louis, Mo.).

For the exemplary protein having a sequence as set forth in SEQ ID NO:2: Absorbance (Ab) at 540 nm over time in minutes at various pHs as indicated is summarized in the graph of FIG. 6A and Relative Activity as a function of pH is summarized in the graph of FIG. 6B. For the exemplary protein having a sequence as set forth in SEQ ID NO:4: Absorbance (Ab) at 540 nm over time in minutes is summarized in the graph of FIG. 6C and Relative Activity as a function of pH is summarized in the graph of FIG. 6D.

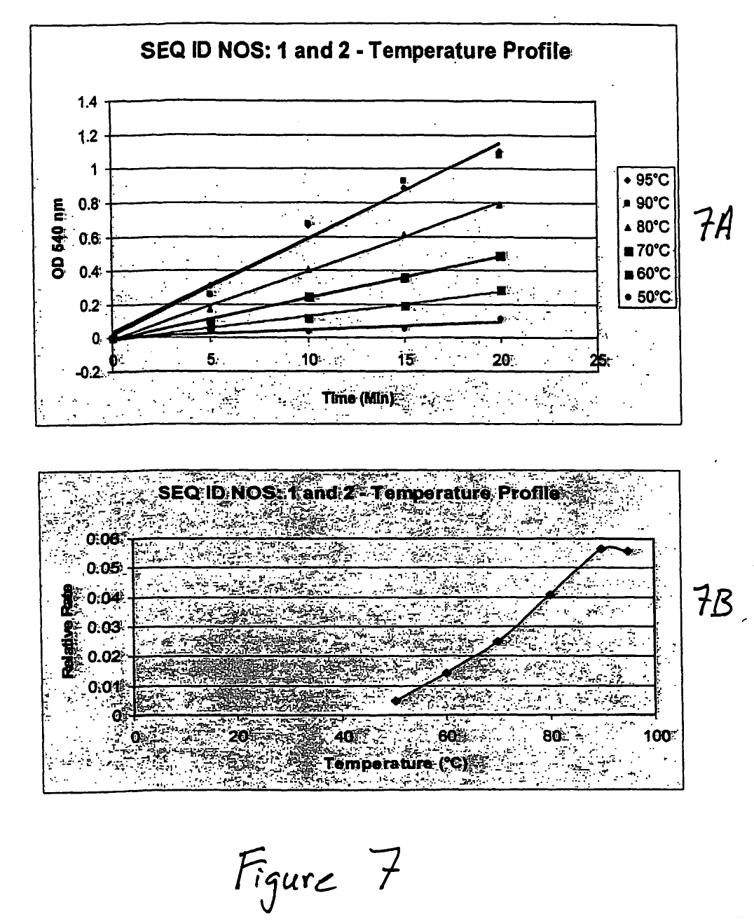

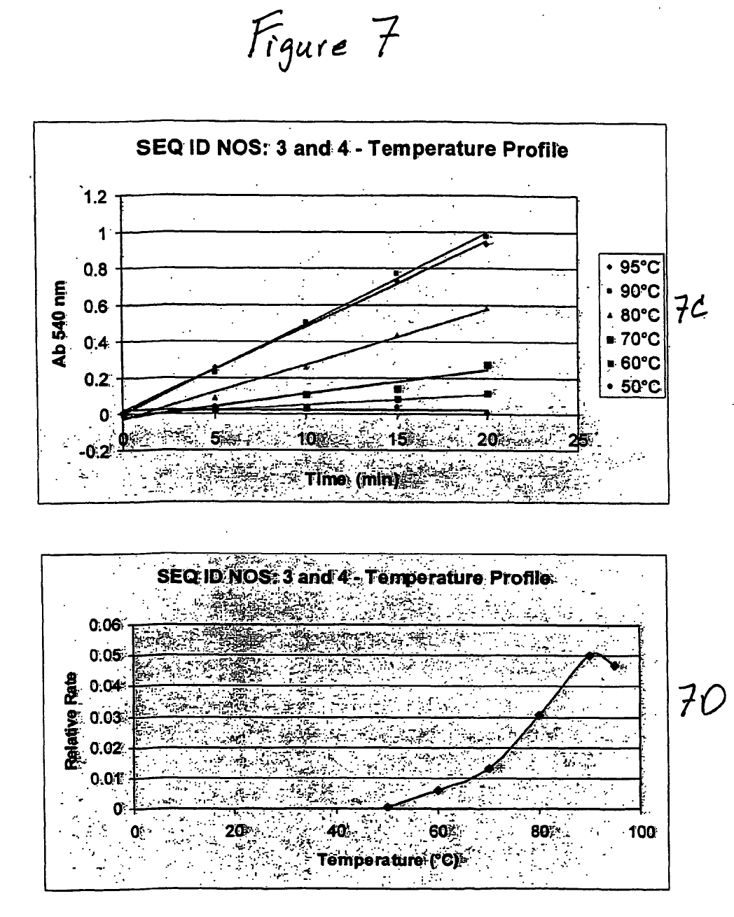

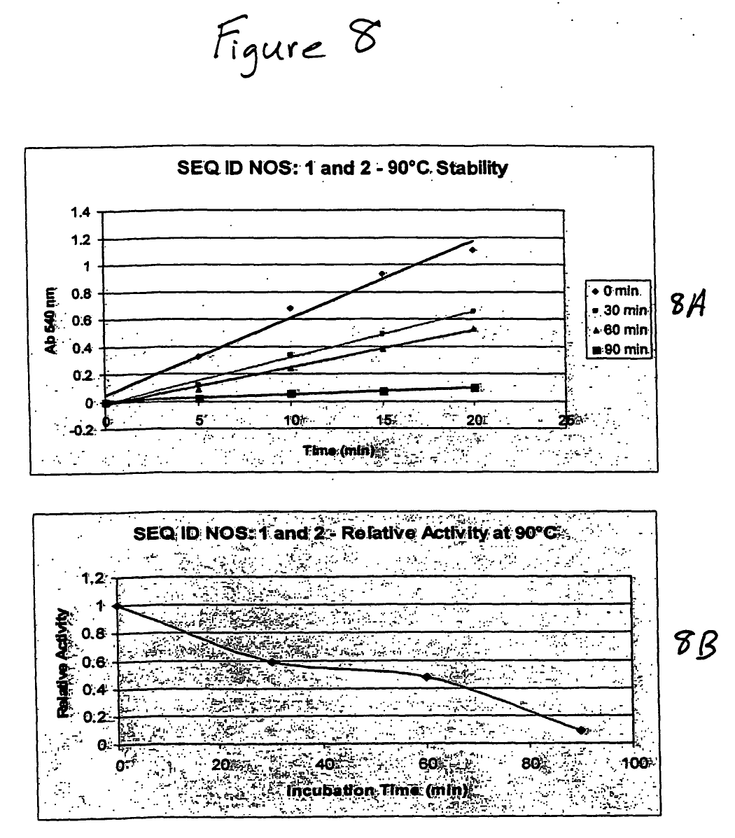

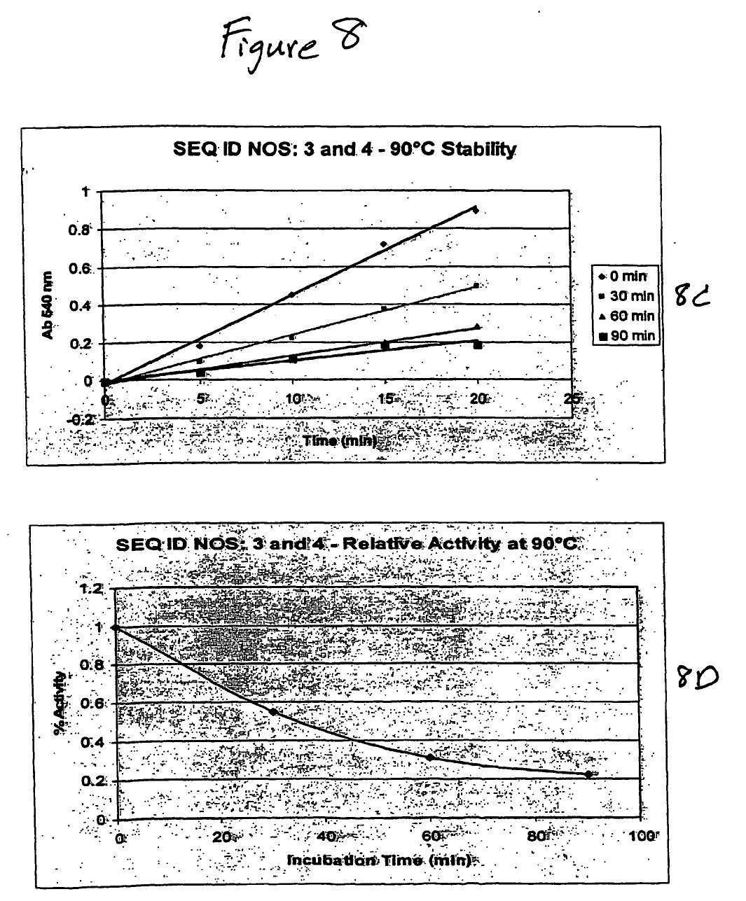

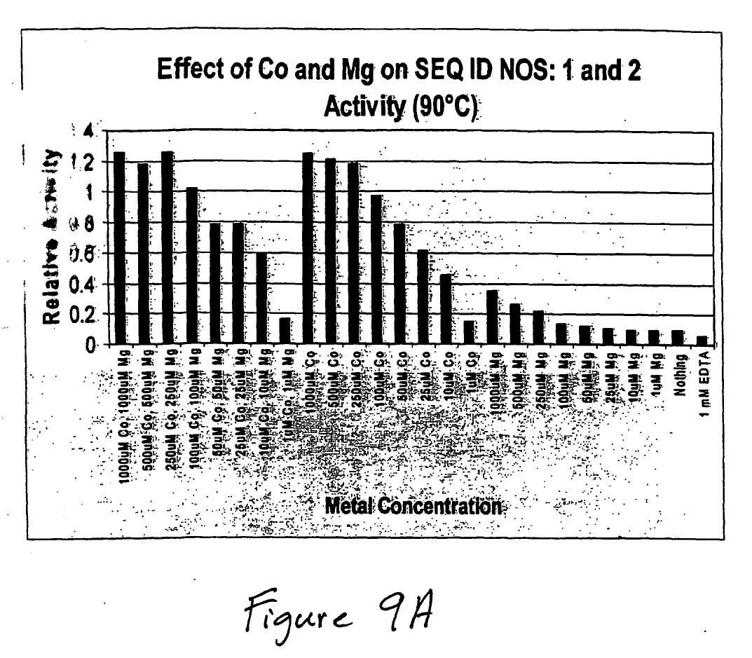

For a series of tests profiling the activities of the exemplary proteins having a sequence as set forth in SEQ ID NO:2 and SEQ ID NO:4 under different temperature conditions, a 20 μl aliquot of resuspended enzyme (having a sequence as set forth in SEQ ID NO:2 or SEQ ID NO:4) was added to a 500 μl reaction buffer (25 mM buffer, pH 6.19, 10 mM fructose, 0.5 mM CoCl₂, 0.5 mM MgCl₂) and held at 50° C. through 95° C., as shown in FIG. 7. 100 μl aliquots were removed to 900 μl 5 mM EDTA on ice at five minute time points. Glucose levels of a 100 μl aliquot were determined from each time point using Sigma's glucose assay kit (Sigma-Aldrich, St. Louis, Mo.).

For the exemplary protein having a sequence as set forth in SEQ ID NO:2: Absorbance (Ab) at 540 nm over time in minutes at various temperatures as indicated is summarized in the graph of FIG. 7A and Relative Activity as a function of temperature is summarized in the graph of FIG. 7B. For the exemplary protein having a sequence as set forth in SEQ ID NO:4: Absorbance (Ab) at 540 nm over time in minutes at various temperatures as indicated is summarized in the graph of FIG. 7C and Relative Activity as a function of temperature is summarized in the graph of FIG. 7D.

For a series of tests profiling the stability of the exemplary proteins having a sequence as set forth in SEQ ID NO:2 and SEQ ID NO:4 over time at 90° C., enzyme lysates were held at 90° C. for up to 90 minutes, as indicated in FIG. 8. A 20 μl aliquot of enzyme (having a sequence as set forth in SEQ ID NO:2 or SEQ ID NO:4) was removed at 30 minute intervals and added to a 500 μl reaction buffer (25 mM buffer, pH 6.19, 10 mM fructose, 0.5 mM CoCl2, 0.5 mM MgCl2) and held at 90° C., as shown in FIG. 8. 100 μl aliquots were removed to 900 μl 5 mM EDTA on ice at five minute time points. Glucose levels of a 100 μl aliquot were determined from each time point using Sigma's glucose assay kit (Sigma-Aldrich, St. Louis, Mo.).

For the exemplary protein having a sequence as set forth in SEQ ID NO:2: Absorbance (Ab) at 540 nm over time in minutes at various time points as indicated is summarized in the graph of FIG. 8A and Relative Activity as a function of incubation time is summarized in the graph of FIG. 8B. For the exemplary protein having a sequence as set forth in SEQ ID NO:4: Absorbance (Ab) at 540 nm over time in minutes at various time points as indicated is summarized in the graph of FIG. 8C and Relative Activity as a function of time is summarized in the graph of FIG. 8D.

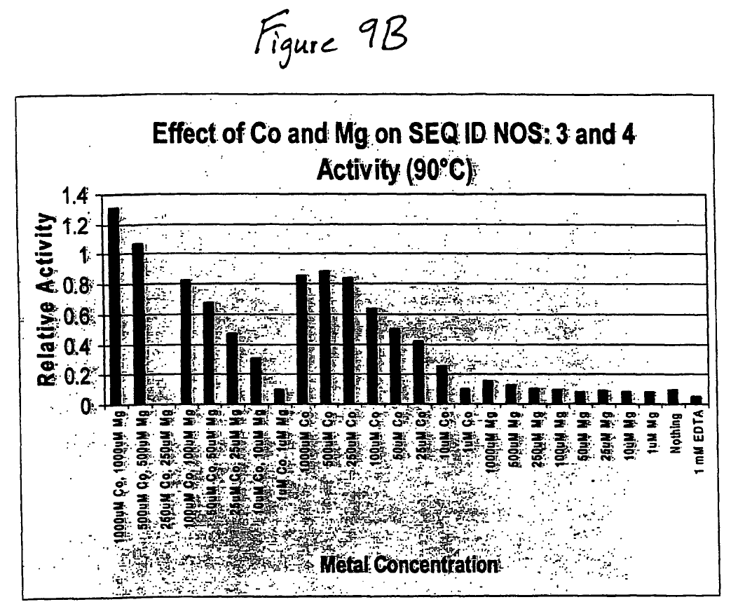

For a series of tests profiling the effect of various metal concentrations of the metals Co and Mg on the activity of the exemplary proteins having a sequence as set forth in SEQ ID NO:2 and SEQ ID NO:4 over time at 90° C., 20 μl aliquots of enzyme (having a sequence as set forth in SEQ ID NO:2 or SEQ ID NO:4) were added to a 400 μl reaction buffer at 90° C., as shown in FIG. 9. Reaction buffer was MOPS at pH 7.12, 10 mM fructose, and the metals as shown in FIG. 9. The reaction proceeded for exactly 20 minutes and was stopped by removing 100 μl aliquots to 900 μl 5 mM EDTA on ice. Glucose levels of a 100 μl aliquot were determined using Sigma's glucose assay kit (Sigma-Aldrich, St. Louis, Mo.).

For the exemplary protein having a sequence as set forth in SEQ ID NO:2: relative activity at various concentrations of Co and Mg as indicated is summarized in the graph of FIG. 9A. For the exemplary protein having a sequence as set forth in SEQ ID NO:4: relative activity at various concentrations of Co and Mg as indicated is summarized in the graph of FIG. 9B.

A number of embodiments of the invention have been described. Nevertheless, it will be understood that various modifications may be made without departing from the spirit and scope of the invention. Accordingly, other embodiments are within the scope of the following claims.

1. An isolated, synthetic or recombinant nucleic acid comprising:
(a) a nucleic acid sequence having at least 96%, 97%, 98%, 99%, or more, or complete (100%) sequence identity to SEQ ID NO:1 over a region of at least about 100, 150, 200, 250, 300, 350, 400, 450, 500, 550, 600, 650, 700, 750, 800, 850, 900, 950, 1000, 1050, 1100, 1150 or more residues, wherein the nucleic acid encodes a polypeptide having a xylose isomerase activity; or
(a) nucleic acid sequence having at least 95%, 96%, 97%, 98%, 99%, or more, or complete (100%) sequence identity to SEQ ID NO:3 over a region of at least about 100, 150, 200, 250, 300, 350, 400, 450, 500, 550, 600, 650, 700, 750, 800, 850, 900, 950, 1000, 1050, 1100, 1150 or more residues, wherein the nucleic acid encodes a polypeptide having a xylose isomerase activity; or
(c) a nucleic acid sequence encoding a polypeptide having a xylose isomerase activity comprising the sequence or SEQ ID NO:2, or SEQ ID NO:4, or enzymatically active fragments thereof; or
(d) a nucleic acid sequence that hybridizes under stringent conditions to a nucleic acid comprising the sequence or SEQ ID NO:1, or SEQ ID NO:3, wherein the nucleic acid encodes a polypeptide having a xylose isomerase activity, wherein the sequence is at least about 25, 30, 35, 40, 45, 50, 55, 60, 70, 80, 90, 100, 125, 150, 175, 200, 225, 250, 275, 300, 325, 350, 400, 450, 500, 550, 600, 650, 700, 750, 800, 850, 900, 950, 1000, 1050, 1100, 1150 or more residues in length, or the full length of a gene or a transcript,
wherein optionally the stringent conditions comprise a wash step comprising a wash in 0.2×SSC at a temperature of about 65° C. for about 15 minutes; or
(e) a nucleic acid sequence completely complementary to (a), (b), (c) or (d),
wherein the nucleic acid encodes at least one polypeptide having a xylose isomerase activity, and optionally the sequence identities are determined by analysis with a sequence comparison algorithm or by a visual inspection,
and optionally the sequence comparison algorithm is a BLAST version 2.2.2 algorithm where a filtering setting is set to blastall -p blastp -d “nr pataa” -F F, and all other options are set to default,
and optionally the xylose isomerase activity comprises: isomerization of xylose to xylulose; isomerization of glucose to fructose; isomerization of D-glucose to D-fructose; catalysis of the conversion of D-xylose to an equilibrium mixture of D-xylulose and D-xylose; isomerization of α-D-glucopyranose to α-D-fructofuranose; isomerization of β-D-glucopyranose to β-D-fructopyranose,
and optionally the xylose isomerase activity is thermostable, and optionally the polypeptide retains a xylose isomerase activity under conditions comprising a temperature range of between about 60° C. to about 120° C., or, between about 60° C. to about 95° C., or retains a xylose isomerase activity under conditions comprising a temperature range of between about 95° C. to about 105° C., or, between about 105° C. to about 120° C.,
and optionally the xylose isomerase activity thermotolerant, and optionally retains a xylose isomerase activity after exposure to conditions comprising a temperature range of between about 95° C. to about 135° C., or, between about 95° C. to about 105° C., or between about 105° C. to about 120° C., or, between about 120° C. to about 135° C.

2-36. (canceled)

37. A nucleic acid probe for identifying, detecting or isolating a nucleic acid encoding a polypeptide comprising a xylose isomerase activity, wherein the probe comprises at least 10, 15, 20, 25, 30, 35, 40, 45, 50, 55, 60, 70, 80, 90, 100, 125, 150, 175, 200, 225, 250, 275, 300, 325, 350, 400, 450, 500, 550, 600, 650, 700, 750, 800, 850, 900, 950, 1000, 1050, 1100, 1150 or more consecutive bases of a sequence comprising: (a) the sequence of claim 1; or,
(b) the sequence of SEQ ID NO:1, or
SEQ ID NO:3,
wherein the probe identifies, detects or isolates the nucleic acid by binding or hybridization,
wherein optionally the probe comprises an oligonucleotide comprising at least about 10 to 50, about 20 to 60, about 30 to 70, about 40 to 80, or about 60 to 100 bases.

38-43. (canceled)

44. An amplification primer pair for amplifying a nucleic acid encoding a polypeptide having a xylose isomerase activity, wherein the primer pair is capable of amplifying a nucleic acid comprising (a) the sequence of claim 1; or,
(b) the sequence of SEQ ID NO:1 or SEQ ID NO:3,
wherein optionally each member of the amplification primer pair comprises an oligonucleotide comprising at least about 10 to 50 consecutive bases of the sequence of (a) or (b), or about 12, 13, 14, 15, 16, 17, 18, 19, 20, 21, 22, 23, 24, 25, 26, 27, 28, 29, 30 or more consecutive bases of the sequence of (a) or (b),
or optionally the primer pair comprises a first member having a sequence as set forth by about the first (the 5′) 12, 13, 14, 15, 16, 17, 18, 19, 20, 21, 22, 23, 24, 25, 26, 27, 28, 29, 30 or more residues of (a) or (b), and a second member having a sequence as set forth by about the first (the 5′) 12, 13, 14, 15, 16, 17, 18, 19, 20, 21, 22, 23, 24, 25, 26, 27, 28, 29, 30 or more residues of the complementary strand of the first member.

45-46. (canceled)

47. An expression cassette, a vector or a cloning vehicle comprising the nucleic acid of claim 1,
wherein optionally the nucleic acid is operably linked to a plant promoter, a potato promoter, a rice promoter, a corn promoter, a wheat promoter or a barley promoter, a promoter derived from T-DNA of Agrobacterium tumefaciens, a constitutive promoter, an inducible promoter, a tissue-specific promoter, a seed-specific, a leaf-specific, a root-specific, a stem-specific or an abscission-induced promoter,
and optionally the expression cassette, vector or cloning vehicle comprises or further comprises a plant expression vector or a plant virus,
and optionally the cloning vehicle comprises a viral vector, a plasmid, a phase, a phagemid, a cosmid, a fosmid, a bacteriophage or an artificial chromosome, and optionally the viral vector comprises an adenovirus vector, a retroviral vector or an adeno-associated viral vector, and optionally the cloning vehicle comprises a bacterial artificial chromosome (BAC), a plasmid, a bacteriophage P1-derived vector (PAC), a yeast artificial chromosome (YAC), or a mammalian artificial chromosome (MAC).

48-60. (canceled)

61. A transformed cell comprising the vector, expression cassette or a cloning vehicle of claim 47, or the nucleic acid of claim 1,
wherein optionally the cell is a bacterial cell, a mammalian cell, a fungal cell, a yeast cell, an insect cell or a plant cell, and optionally the plant cell is a potato, rice, corn, wheat, tobacco, rapeseed, grass, soybean or barley cell.

62-64. (canceled)

65. A transgenic non-human animal or transgenic plant or seed comprising
the nucleic acid sequence of claim 1, or the vector, expression cassette or a cloning vehicle of claim 47,
wherein optionally the animal is a mouse,
and optionally the plant is a corn plant, a potato plant, a grass, a tomato plant, a wheat plant, an oilseed plant, a rapeseed plant, a soybean plant or a tobacco plant,
and optionally the seed is a starch granule or grain, corn seed, a wheat kernel, an oilseed, a rapeseed, a soybean seed, a palm kernel, a sunflower seed, a sesame seed, a peanut or a tobacco plant seed.

66-71. (canceled)

72. An antisense oligonucleotide comprising a nucleic acid sequence complementary to or capable of hybridizing under stringent conditions to
the nucleic acid sequence of claim 1,
wherein optionally the antisense oligonucleotide is between about 10 to 50, about 20 to 60, about 30 to 70, about 40 to 80, or about 60 to 100 bases in length.

73-74. (canceled)

75. An isolated, synthetic or recombinant polypeptide comprising
(a) a polypeptide comprising
an amino acid sequence having at least 96%, 97%, 98%, 99%, or more, or complete (100%) identity to SEQ ID NO:2 over a region of at least about 25, 50, 75, 100, 125, 150, 175, 200, 225, 250, 275, 300, 325, 350 or more residues, or over the full length of the polypeptide, or
an amino acid sequence having at least 95%, 96%, 97%, 98%, 99%, or more, or complete (100%) identity to SEQ ID NO:4 over a region of at least about 25, 50, 75, 100, 125, 150, 175, 200, 225, 250, 275, 300, 325, 350 or more residues, or over the full length of the polypeptide, or
(b) a polypeptide encoded by the nucleic acid of claim 1;
(c) the polypeptide of (a) or (b) lacking a signal sequence; or
(d) the polypeptide of (a), (b) or (c) comprising a heterologous signal sequence or catalytic domain (CD), wherein optionally the signal sequence comprises a heterologous xylose isomerase or non-xylose isomerase signal sequence,
wherein the nucleic acid encodes at least one polypeptide having a xylose isomerase activity, and optionally the sequence identities are determined by analysis with a sequence comparison algorithm or by a visual inspection,
and optionally the sequence comparison algorithm is a BLAST version 2.2.2 algorithm where a filtering setting is set to blastall -p blastp -d “nr pataa” -F F, and all other options are set to default,
and optionally the xylose isomerase activity comprises: isomerization of xylose to xylulose; isomerization of glucose to fructose; isomerization of D-glucose to D-fructose; catalysis of the conversion of D-xylose to an equilibrium mixture of D-xylulose and D-xylose; isomerization of α-D-glucopyranose to α-D-fructofuranose; isomerization of β-D-glucopyranose to β-D-fructopyranose,
and optionally the xylose isomerase activity is thermostable, and optionally the polypeptide retains a xylose isomerase activity under conditions comprising a temperature range of between about 60° C. to about 120° C., or, between about 60° C. to about 95° C., or retains a xylose isomerase activity under conditions comprising a temperature range of between about 95° C. to about 105° C., or, between about 105° C. to about 120° C.,
and optionally the xylose isomerase activity thermotolerant and optionally retains a xylose isomerase activity after exposure to conditions comprising a temperature range of between about 95° C. to about 135° C., or, between about 95° C. to about 105° C., or between about 105° C. to about 120° C., or, between about 120° C. to about 135° C.,
and optionally the xylose isomerase activity comprises a specific activity at about 95° C. in the range from about 100 to about 1000 units per milligram of protein, or, a specific activity from about 500 to about 750 units per milligram of protein, or, a specific activity at 95° C. in the range from about 500 to about 1200 units per milligram of protein, or, a specific activity at 95° C. in the range from about 750 to about 1000 units per milligram of protein,
and optionally the polypeptide comprises at least one glycosylation site, and optionally the glycosylation is an N-linked glycosylation, and optionally the polypeptide is glycosylated after being expressed in a P. pastoris or a S. pombe,
and optionally the polypeptide retains a xylose isomerase activity under conditions comprising about pH 4.0, 4.5, 5.0, 5.5, 6.0 or 6.5, or the polypeptide retains a xylose isomerase activity under conditions comprising about pH 8.0, 8.5, 9.0, 9.5, 10, 10.5 or 11.

76-98. (canceled)

99. An isolated, synthetic or recombinant signal sequence peptide comprising a sequence as set forth in the amino terminal 20 to 30 residues of (a) the polypeptide of claim 75, or (b) the polypeptide having the amino acid sequence of SEQ ID NO:2 or SEQ ID NO:4.

100-105. (canceled)

106. A protein preparation comprising a polypeptide as set forth in claim 75, wherein the protein preparation comprises a liquid, a solid or a gel.

107. A homodimer comprising a polypeptide as set forth in claim 75.

108. A heterodimer comprising a polypeptide as set forth in claim 75 and a second domain, wherein optionally the second domain is a polypeptide and the heterodimer is a fusion protein, or the second domain is an epitope or a tag.

109-111. (canceled)

112. An immobilized polypeptide having a xylose isomerase activity, wherein the polypeptide comprises a sequence as set forth in claim 75 or claim 108, wherein optionally the polypeptide is immobilized on a cell, a metal, a resin, a polymer, a ceramic, a glass, a microelectrode, a graphitic particle, a bead, a gel, a plate, an array or a capillary tube.

113. (canceled)

114. An array comprising an immobilized polypeptide asset forth in claim 75 or claim 108, or an immobilized nucleic acid asset forth in claim 1.

115. (canceled)

116. An isolated or recombinant antibody that specifically binds to the polypeptide of claim 75 or to a polypeptide encoded by the nucleic acid of claim 1, wherein optionally the antibody is a monoclonal or a polyclonal antibody.

117. (canceled)

118. A hybridoma comprising an antibody that specifically binds to the polypeptide of claim 75 or to a polypeptide encoded by the nucleic acid of claim 1.

119. A food supplement for an animal comprising the polypeptide of claim 75 or to a polypeptide encoded by the nucleic acid of claim 1,
wherein optionally the polypeptide is glycosylated, or the food supplement comprises a glucose or a starch.

120-121. (canceled)

122. An edible enzyme delivery matrix comprising the polypeptide of claim 75 or to a polypeptide encoded by the nucleic acid of claim 1, wherein optionally the polypeptide comprises a xylose isomerase activity, or the edible enzyme delivery matrix comprises a glucose or a starch, or the delivery matrix comprises a pellet, or the polypeptide is glycosylated, or the xylose isomerase activity is thermotolerant or thermostable.

123-128. (canceled)

129. A method of producing a recombinant polypeptide comprising the steps of
(a) providing a nucleic acid operably linked to a promoter; wherein the nucleic acid comprises the nucleic acid sequence of claim 1; and
(b) expressing the nucleic acid of step (a) under conditions that allow expression of the polypeptide, thereby producing a recombinant polypeptide,
and optionally the method further comprises transforming a host cell with the nucleic acid of step (a) followed by expressing the nucleic acid of step (a), thereby producing a recombinant polypeptide in a transformed cell, and optionally the cell is a plant cell.

130-139. (canceled)

140. A computer system comprising a processor and a data storage device wherein said data storage device has stored thereon a polypeptide sequence or a nucleic acid sequence, wherein the polypeptide sequence comprises the sequence of claim 75, or subsequence thereof, and the nucleic acid comprises the nucleic acid sequence of claim 1, or subsequence thereof,
and optionally the computer system further comprises a sequence comparison algorithm and a data storage device having at least one reference sequence stored thereon, or optionally the sequence comparison algorithm comprises a computer program that indicates polymorphisms, or optionally the computer system further comprises an identifier that identifies one or more features in said sequence.

141-143. (canceled)

144. A computer readable medium having stored thereon a polypeptide sequence or a nucleic acid sequence, wherein the polypeptide sequence comprises the sequence of claim 75, or subsequence thereof, and the nucleic acid comprises the nucleic acid sequence of claim 1, or subsequence thereof.

145-149. (canceled)

150. A method for isolating, detecting or recovering a nucleic acid encoding a polypeptide with a xylose isomerase activity from a sample comprising the steps of:
(A) (a) providing an amplification primer sequence pair for amplifying a nucleic acid encoding a polypeptide with a xylose isomerase activity, wherein the primer pair is capable of amplifying SEQ ID NO:1 or SEQ ID NO:3, or a subsequence thereof, or the sequence of claim 1;
(b) isolating a nucleic acid from the sample or treating the sample such that nucleic acid in the sample is accessible for hybridization to the amplification primer pair; and,
(c) combining the nucleic acid of step (b) with the amplification primer pair of step (a) and amplifying nucleic acid from the sample, thereby isolating, detecting or recovering a nucleic acid encoding a polypeptide with a xylose isomerase activity from a sample; or,
(B) (a) providing a polynucleotide probe comprising the sequence of claim 1, or the probe sequence of claim 37;
(b) isolating a nucleic acid from the sample or treating the sample such that nucleic acid in the sample is accessible for hybridization to a polynucleotide probe of step (a);
(c) combining the isolated nucleic acid or the treated sample of step (b) with the polynucleotide probe of step (a); and
(d) isolating a nucleic acid that specifically hybridizes with the polynucleotide probe of step (a), thereby isolating, detecting or recovering a nucleic acid encoding a polypeptide with a xylose isomerase activity from the sample,
wherein optionally the sample comprises an environmental sample, and optionally each member of the amplification primer sequence pair comprises an oligonucleotide comprising at least about 10 to 50 consecutive bases of a sequence as set forth in SEQ ID NO:1, SEQ ID NO:3, or a subsequence thereof,
and optionally the environmental sample comprises a water sample, a liquid sample, a soil sample, an air sample or a biological sample, and optionally the biological sample is derived from a bacterial cell, a protozoan cell, an insect cell, a yeast cell, a plant cell, a fungal cell or a mammalian cell.

151-154. (canceled)

155. A method of generating a variant of a nucleic acid encoding a polypeptide with a xylose isomerase activity comprising the steps of:
(a) providing a template nucleic acid comprising a sequence as set forth in claim 1 or claim 30; and
(b) modifying, deleting or adding one or more nucleotides in the template sequence, or a combination thereof, to generate a variant of the template nucleic acid,
wherein optionally the method further comprises expressing the variant nucleic acid to generate a variant xylose isomerase polypeptide, or optionally the modifications, additions or deletions are introduced by a method comprising error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturation mutagenesis (GSSM), synthetic ligation reassembly (SLR), recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, or a combination thereof,
and optionally the method is iteratively repeated until a xylose isomerase having an altered or different activity or an altered or different stability from that of a polypeptide encoded by the template nucleic acid is produced, or optionally the variant xylose isomerase polypeptide is thermotolerant, and retains some activity after being exposed to an elevated temperature, or optionally the variant xylose isomerase polypeptide has increased glycosylation as compared to the xylose isomerase encoded by a template nucleic acid, or optionally the variant xylose isomerase polypeptide has a xylose isomerase activity under a high temperature, wherein the xylose isomerase encoded by the template nucleic acid is not active under the high temperature,
and optionally the method is iteratively repeated until a xylose isomerase coding sequence having an altered codon usage from that of the template nucleic acid is produced,
and optionally the method is iteratively repeated until a xylose isomerase gene having higher or lower level of message expression or stability from that of the template nucleic acid is produced.

156-177. (canceled)

178. A method for modifying codons in a nucleic acid encoding a polypeptide with a xylose isomerase activity to increase its expression in a host cell, the method comprising the following steps:
(A) (a) providing a nucleic acid encoding a polypeptide with a xylose isomerase activity comprising the nucleic acid sequence of claim 1, or a nucleic acid encoding the polypeptide of claim 75; and,
(b) identifying a non-preferred or a less preferred codon in the nucleic acid of step (a) and replacing it with a preferred or neutrally used codon encoding the same amino acid as the replaced codon, wherein a preferred codon is a codon over-represented in codon under-represented in coding sequences in genes in the host cell, thereby modifying the nucleic acid to increase its expression in a host cell, or
(B) (a) providing a nucleic acid encoding a polypeptide with a xylose isomerase activity comprising the sequence of claim 1, or a nucleic acid encoding the polypeptide of claim 75; and,
(b) identifying a codon in the nucleic acid of step (a) and replacing it with a different codon encoding the same amino acid as the replaced codon thereby modifying codons in a nucleic acid encoding a xylose isomerase,
wherein optionally the host cell is a bacterial cell, a fungal cell, an insect cell, a yeast cell, a plant cell or a mammalian cell.

179-203. (canceled)

204. A kit comprising the polypeptide of claim 75 or a polypeptide encoded by the nucleic acid of claim 1, wherein the polypeptide comprises a xylose isomerase activity.

205. A method for catalyzing the isomerization of a glucose to a fructose comprising the following steps:
(a) providing the polypeptide of claim 75 or a polypeptide encoded by the nucleic acid of claim 1, wherein the polypeptide comprises a xylose isomerase activity;
(b) providing a composition comprising a glucose; and
(c) contacting the polypeptide of step (a) with the glucose of step (b) under conditions wherein the polypeptide of step (a) can isomerase the glucose to a fructose, thereby producing a fructose.

206. A method for producing fructose from a starch comprising the following steps:
(a) providing a polypeptide capable of hydrolyzing a α-1,4-glycosidic linkage in a starch;
(b) contacting the polypeptide of the step (a) with the starch under condition wherein the polypeptide of step (a) can hydrolyze α-1,4-glycosidic linkages in the starch,
(c) providing the polypeptide of claim 75 or a polypeptide encoded by the nucleic acid of claim 1, wherein the polypeptide comprises a xylose isomerase activity; and
(d) contacting the polypeptide of step (c) with the glucose of step (b) under conditions wherein the polypeptide of step (c) can isomerase glucose, thereby producing fructose,
wherein optionally the polypeptide of step (a) comprises an xylose isomerase or a glucoamylase, and optionally step (a) further comprises or comprises a polypeptide capable of hydrolyzing α-1,6-glycosidic linkage in a starch.

207-208. (canceled)

209. A method for producing fructose comprising the following steps:
(a) providing a glucose;
(b) providing a polypeptide having a xylose isomerase activity, wherein the polypeptide comprises the amino acid sequence of claim 75, or, a polypeptide encoded by the nucleic acid having a sequence of claim 1; and
(c) contacting the polypeptide of step (b) with the glucose of step (a) under conditions wherein the polypeptide can isomerase glucose thereby producing fructose,
wherein optionally the conditions comprise a temperature of between about 70° C. and 95° C., thereby shifting equilibrium of the reaction towards formation of fructose, and optionally the conditions comprise a temperature of between about 80° C. and 90° C., thereby shifting equilibrium of the reaction towards formation of fructose, and optionally the polypeptide of step (b) is immobilized.

210-212. (canceled)

213. A method of making fructose in a feed or a food prior comprising the following steps:
(a) obtaining a feed or a food material comprising a starch,
(b) providing a polypeptide capable of hydrolyzing a α-1,4-glycosidic linkage in a starch;
(c) contacting the polypeptide of the step (a) with the feed or a food material under conditions wherein the polypeptides of step (a) can hydrolyze α-1,4-glycosidic linkages in the starch to produce a glucose;
(d) providing the polypeptide of claim 75 or a polypeptide encoded by the nucleic acid of claim 1, wherein the polypeptide comprises a xylose isomerase activity; and
(e) adding the polypeptide of step (d) to the feed or food material in an amount sufficient to cause isomerization of the glucose to a fructose in the food or the feed,
wherein optionally the food or feed comprises rice, corn, barley, wheat, legumes, or potato, and optionally step (a) further comprises a polypeptide capable of hydrolyzing α-,1,6-glycosidic linkage in a starch.

214-215. (canceled)

216. A method for producing a high-fructose syrup comprising the following steps:
(a) providing a polypeptide capable of hydrolyzing α-1,4-glycosidic linkages in a starch;
(b) providing a composition comprising a starch
(c) contacting the polypeptides of step (a) and the composition of step (b) under conditions wherein the polypeptide of step (a) can hydrolyze α-1,4-glycosidic linkages in the starch;
(d) providing the polypeptide of claim 75 or a polypeptide encoded by the nucleic acid of claim 1, wherein the polypeptide comprises a xylose isomerase activity; and
(e) contacting the polypeptide of step (d) and the starch hydrolysate of step (c) under conditions wherein the polypeptide of step (d) can isomerase glucose in the starch hydrolysate to a fructose, thereby producing the high-fructose syrup,
wherein optionally the composition comprises a rice, a corn, a barley, a wheat, a legume, a potato or a sweet potato, and optionally the composition comprises a rice and the high-fructose syrup is a high-fructose corn syrup, and optionally step (a) further comprises a polypeptide capable of hydrolyzing α-1,6-glycosidic linkage in a starch, and optionally all reactions are carried out in one vessel, and optionally the high-fructose syrup comprises an insecticide bait composition.

217-221. (canceled)

222. A method for producing a high-fructose syrup comprising the following steps:
(a) providing a transgenic seed or grain comprising the polypeptide of claim 75 or a polypeptide encoded by the nucleic acid of claim 1, comprising a xylose isomerase activity, wherein the seed or grain comprises a starch;
(b) expressing the xylose isomerase in the seed or grain;
(c) hydrolyzing the starch to a glucose under conditions wherein the polypeptide of step (a) expressed in the seed or grain can catalyze isomerization of glucose to a fructose, thereby producing the high-fructose syrup,
wherein optionally the steps of hydrolyzing the starch and isomerizing the glucose are carried out at pH 4.0 to 6.5 and at temperature comprising a range of about 55° C. to 105° C.

223. (canceled)

224. A method for producing fructose in brewing or alcohol production comprising the following steps:
(a) providing the polypeptide of claim 75 or a polypeptide encoded by the nucleic acid of claim 1, wherein the polypeptide comprises a xylose isomerase activity;
(b) providing malt or mash composition comprising a glucose; and
(c) contacting the polypeptide of step (a) with the composition of step (b) under conditions wherein the polypeptide of step (a) isomerizes the glucose of step (b) to a fructose, thereby producing fructose for brewing or alcohol production.

225-228. (canceled)

229. A double-stranded inhibitory RNA (RNAi) molecule comprising a subsequence of the nucleic acid of claim 1, wherein optionally the RNAi is about 15, 16, 17, 18, 19, 20, 2-1, 22, 23, 24, 25 or more duplex nucleotides in length.

230-234. (canceled)

235. A recombinant xylose isomerase-encoding nucleic acid generated by amplification of the polynucleotide of claim 1 using the amplification primer pair of claim 44, wherein optionally the amplification is by polymerase chain reaction (PCR), or optionally the nucleic acid generated by amplification of a gene library, or optionally the gene library is an environmental library.

236-238. (canceled)

239. An isolated, synthetic or recombinant protease encoded by the xylose isomerase-encoding nucleic acid sequence of claim 235.


SEQUENCE LISTING (4 sequences)

SEQ ID NO: 1 - DNA, 1335 bp, unknown
  obtained from an environmental sample
atgactgagttctttccagagatcccgaagatacagtttgaaggtaaagagagcacaaatccatttgcgttcaagttctacgatccaaacgaggtgatcgacggaaaacctctcaaggaccatctgaagttctcagttgcattctggcacaccttcgtgaacgaggggagagatcccttcggagatccaacagccgaccgaccctggaacaagtacacagaccctatggacaaagcctttgcaagggtggacgccctctttgaattctgtgaaaaactcaacatcgagtacttctgttttcacgacagggacatagctcctgaaggaaagactctgagggagacaaacaagatcctggacaaggtcgtggagaggatcaaagagagaatgaaagacagcaacgtaaaactcctctggggtactgcgaatctcttttctcatccaaggtacatgcacggtgcggcgacaacctgtagtgctgatgtcttcgcctacgcggcagcacaggtgaagaaagcccttgagatcacaaaagagcttggaggagaagggtacgtcttttggggtggaagagaagggtacgagacactcctcaacacggatctggatcttgaacttggaaacctcgctcgcttcctcagaatggctgtggattacgcaaagaagataggtttcaacggccagtttctcatcgagcctaaaccgaaggaaccaacgaagcatcagtacgacttcgatgttgcgacggcttacgccttcctgaagagtcacggtctcgatgagtatttcaaattcaacatcgaagcgaaccatgccacacttgctggtcacaccttccagcacgaactgaggatggcaagaattcttggaaaactcggcagcatcgacgcgaaccagggggaccttctgctcggctgggacaccgaccagttcccaacaaacgtctacgacacaactcttgccatgtatgaagtgataaaagcgggtgggtttacaaaaggtggtctcaacttcgatgcaaaggtgagaagagcttcttacaaggtggaagatctcttcatcgggcacatagcaggaatggatactttcgcactcggtttcaaaatagcccacaaacttgtaaaagacggtgtgttcgacaagttcattgaagaaaaatacaaaagtttcagagagggcatcggaaaagagatcgttgaaggaaaggcagattttgaaaagctggaagcttatataatagacaaggaagagatggagcttccatctggaaagcaggagtatttggaaagtctcctcaacagctacatagtgaaaacgatctccgagttgaggtga

SEQ ID NO: 2 - protein, 444 aa, unknown
  obtained from an environmental sample
MTEFFPEIPKIQFEGKESTNPFAFKFYDPNEVIDGKPLKDHLKFSVAFWHTFVNEGRDPFGDPTADRPWNKYTDPMDKAFARVDALFEFCEKLNIEYFCFHDRDIAPEGKTLRETNKILDKVVERIKERMKDSNVKLLWGTANLFSHPRYMHGAATTCSADVFAYAAAQVKKALEITKELGGEGYVFWGGREGYETLLNTDLDLELGNLARFLRMAVDYAKKIGFNGQFLIEPKPKEPTKHQYDFDVATAYAFLKSHGLDEYFKFNIEANHATLAGHTFQHELRMARILGKLGSIDANQGDLLLGWDTDQFPTNVYDTTLAMYEVIKAGGFTKGGLNFDAKVRRASYKVEDLFIGHIAGMDTFALGFKIAHKLVKDGVFDKFIEEKYKSFREGIGKEIVEGKADFEKLEAYIIDKEEMELPSGKQEYLESLLNSYIVKTISELR

SEQ ID NO: 3 - DNA, 1335 bp, unknown
  obtained from an environmental sample
atgacagaatttttcccggaaattccaaagatacagttcgaagggaaggaaagcaataaccctcttgcctttaagttctacgatccagacgaagtaatcgatggaaaacctctgaaggaccatttgaaattctccgttgctttctggcacacttttgtaaacgaaggtcgagatcccttcggtgaccccactgctgaaagaccctggaacaagtattcggatcccatggacaaagcgtttgcaagagtggatgctttattcgaattctgtgagaaactcaatattgaatacttttgttttcatgacagagacattgcacccgaagggaaaactctgagagagacgaacaaaattctggacaaagttgttgagaaaataaaagaacgaatgaaggaaagcaatgtgaaactcctttggggaactgccaatctgttctcacatcctcggtacatgcacggtgcggcaactacttgcagcgccgatgtttttgcatacgctgctgcacaggtgaaaaaagcgttggagattacgaaggaacttggaggagaaggatatgttttttggggcggtagagaaggatacgaaaccttgctcaacacggatttgggattggaactcgaaaacctcgcgaggttcctcagaatggccgtagagtacgcaaagaagataggttttgatggacagttcctcatagaacccaaaccaaaagaacccacaaaacatcagtacgatttcgacgtagcgaccgcatacgccttcttgaaaactcacgatttggatgaatacttcaagttcaacatagaagctaatcacgcaacactcgctggtcatactttccagcatgaattgagaatggccagaatcctcggaaaattcggaagtatcgacgcaaatcaaggcgatcttctgttgggatgggacaccgatcaatttccaacgaacgtatacgatacaactcttgccatgtacgaggttataaaagcagggggtttcacaaaaggtggtctcaacttcgacgccaaagtgagacgtgcttcttacaaggtagaggatctcttcatcgggcatatagtaggaatagacactttcgcactcggtttcaagatagcctacaaacttgtaaaagacggcgtattcgacagattcgttgaggaaaaatacagaagtttcagagaaggtattggaaaagaaatattggaaggaaaagcagattttgaaaaactagaatcgtatataatagacaaagaagatgttgaacttccatctggaaaacaggagtatcttgaaagtttgctcaacagctatatcgtgaagaccgtatcagaactgaggtga

SEQ ID NO: 4 - protein, 444 aa, unknown
  obtained from an environmental sample
MTEFFPEIPKIQFEGKESNNPLAFKFYDPDEVIDGKPLKDHLKFSVAFWHTFVNEGRDPFGDPTAERPWNKYSDPMDKAFARVDALFEFCEKLNIEYFCFHDRDIAPEGKTLRETNKILDKVVEKIKERMKESNVKLLWGTANLFSHPRYMHGAATTCSADVFAYAAAQVKKALEITKELGGEGYVFWGGREGYETLLNTDLGLELENLARFLRMAVEYAKKIGFDGQFLIEPKPKEPTKHQYDFDVATAYAFLKTHDLDEYFKFNIEANHATLAGHTFQHELRMARILGKFGSIDANQGDLLLGWDTDQFPTNVYDTTLAMYEVIKAGGFTKGGLNFDAKVRRASYKVEDLFIGHIVGIDTFALGFKIAYKLVKDGVFDRFVEEKYRSFREGIGKEILEGKADFEKLESYIIDKEDVELPSGKQEYLESLLNSYIVKTVSELR